(19) United States
(12) Patent Application Publication
(10) Pub. No.: US 2025/0382382 A1
(43) Pub. Date: Dec. 18, 2025

(54) ORTHOGONAL GPC3 CHIMERIC ANTIGEN RECEPTOR T CELLS

(71) Applicant: Synthekine, Inc., Menlo Park, CA (US)

(72) Inventors: Paul-Joseph Penaflor-Aspuria, Menlo Park, CA (US); Deepti Rokkam, Menlo Park, CA (US)

(21) Appl. No.: 18/843,379

(22) PCT Filed: Mar. 7, 2023
(86) PCT No.: PCT/US2023/063868

§ 371 Date: Sep. 3, 2024

Related U.S. Application Data
(60) Provisional application No. 63/317,935, filed on Mar. 8, 2022.

(51) Int. Cl. C07K 16/30; A61K 38/20; A61K 40/11; A61K 40/31; A61K 40/42; A61P 35/00; C07K 14/705; C07K 14/715; C07K 14/725; C12N 5/0783; C12N 15/86

(52) U.S. Cl. CPC C07K 16/303; A61K 38/2013; A61K 40/11; A61K 40/31; A61K 40/4261; A61P 35/00; C07K 14/7051; C07K 14/70521; C07K 14/7155; C12N 5/0636; C12N 15/86; C07K 2317/53; C07K 2317/569; C07K 2317/622; C07K 2319/02; C07K 2319/03; C12N 2510/00; C12N 2740/15043

(57) ABSTRACT

Engineered T cells are provided that express (a) a chimeric antigen receptor wherein the antigen binding domain of the CAR binds to human GPC3 (“a GPC-CAR”); and (b) an orthogonal receptor. Also provided are methods of making and using such engineered T cells.

### CROSS-REFERENCES TO RELATED APPLICATIONS

The present patent application is a U.S. National Stage of PCT/US2023/063868, international filing date Mar. 7, 2023 which claims benefit of priority to U.S. Provisional Patent Application No. 63/317,935, filed Mar. 8, 2022, which is incorporated by reference for all purposes.

### SEQUENCE LISTING

The contents of the electronic sequence listing (106249-1373929-007410WO_ST26.xml; Size: 106,496 bytes; and Date of Creation: Aug. 19, 2024) is herein incorporated by reference in its entirety.

### BACKGROUND OF THE INVENTION

Glypican-3 (GPC3) is a member of the glypican family of heparan surface proteoglycans. GPC3 is also referred to as DGSX, GTR2-2, MXR7, OCI-5, SDYS, SGB, SGBS, and SGBS1. The human GPC3 (hGPC3) gene encodes a 580 amino acid precursor protein (SEQ ID NO:1) comprising a 24 amino acid signal peptide (corresponding to amino acids 1-24 of SEQ ID NO:1) sequence and a C-terminal sequence corresponding to amino acids 555-580 of SEQ ID NO:1, both of which are removed from the mature form of the protein. Two heparan sulfates are linked at amino acid positions 495 and 508. hGPC3 contains a furin cleavage site between amino acids R359 and S359 (numbered in accordance with SEQ ID NO:1) providing an N-terminal 40 kDa fragment having a sequence corresponding to amino acids 25-358 of SEQ ID NO: 1 and the C-terminal 30 kDa fragment having a sequence corresponding to amino acids 359-554 of SEQ ID NO:1 (SEQ ID NO:2). When expressed on the cell surface following furin cleavage, the 40 kDa and 30-kDda fragments h are linked via three disulfide bonds. Hydrolysis of these disulfide bonds liberates the 40 kDa subunit which is referred to as “soluble GPC3” or “sGPC3” which is detectable in the serum and the level of GPC3 is suggested as a diagnostic marker for the estimation of GPC3 expression level in the subject and a marker for the presence of GPC3 expressing tumors such as hepatocellular carcinoma (HCC). GPC3 attached to the cell surface by a glycosylphosphatidylinositol (GPI) anchor.

GPC3 is rarely expressed in normal tissue and multiple studies have identified GPC3 as a cancer specific target, particularly as a liver cancer-specific target, because it is highly expressed in HCC. Baumhoer D, et al. (2008) Am J Clin Pathol 129 (6): 899-906. In addition, GPC3 is rarely expressed in other normal tissues of adults, and therefore is suitable for targeted therapy as a tumor antigen. Li et al (2018) Trends in cancer 4:741-54; Ho and Kim (2011) European journal of cancer 47:333-8 Although initially associated with liver cancer (HCC in particular) a variety of neoplasms characterized by GPC3 expression include but are not limited hepatoblastoma (Zhou S, et al (2017) Scientific reports 7:45932), lung squamous cell carcinoma (Li et al (2016) Oncotarget 7:2496-507)′, ovarian yolk sac tumor (Esheba et al. (2008) American journal of surgical pathology; 32:600-7), melanoma (Nakatsura and Nishimura (2005) BioDrugs: clinical immunotherapeutics, biopharmaceuticals and gene therapy 19:71-7), clear cell carcinoma of the ovary (Umezu (2010) Journal of clinical pathology 63:962-6. Therefore, GPC3 is not only a specific biomarker and prognostic factor for HCC, but also a potential target for a variety of tumor treatments. Given the correlation with GPC3 in a wide variety of tumors, there are numerous GPC-3 CAR T cell therapies in development including multiple clinical trials (see e.g., ClinicalTrials.gov trial numbers NCT02395250, NCT02723942, NCT03146234, NCT0295188, NCT03084380, and NCT03884751). These trials evaluated the effects of GPC3 CAR-T therapy alone or in conjunction with the administration of other anti-cancer agents such as checkpoint inhibitors.

At the present time, the only CAR-T cell agents approved for use by regulatory authorities are targeted at CD19 expressing cells and are used in the treatment of hematological malignancies. There is no approved CAR-T cell therapy for the treatment of solid tumors. The development of CAR-T cells has shown significant growth in recent years with multiple combinations of technologies resulting in what are referred to as 1^{st}, 2^{nd}, 3^{rd} and 4^{th} generation CAR-T cells. Although clinical experience with CAR-T cells for the treatment of hematologic malignancies has shown significant initial success, over time there is a substantial rate of disease recurrence. What is termed “persistence” of CAR-T cells is a particular hurdle to existing technologies. It is well established that adoptively transferred human immune cells lose their activity relatively rapidly following administration. Consequently, the typical means to address this rapid loss of function are: (a) administration excessively high doses of the cell therapy agent to maximize the exposure of the cell therapy agent to the tumor before the cells lose effectiveness, and/or (b) systemic administration of high dose IL2 (HD-hIL2) therapy to attempt to support the efficacy of the adoptively transferred cell. Both of these approaches present significant toxicity.

The effect of high dose hIL2 such as that used in support of adoptive cell therapy regimens is documented to result in significant toxicities in human subjects. The most prevalent side effects observed from the administration of HD-hIL2 in conjunction with adoptive cell transfer (ACT) include chills, high fever, hypotension, oliguria, and edema due to the systemic inflammatory and capillary leak syndrome as well as reports of autoimmune phenomena such as vitiligo or uveitis. HD-hIL2 monotherapy may also induce generalized capillary leak syndrome which can lead to death. The toxicities associated with HD-hIL2 require expert management and is therefore typically applied in the hospital setting and frequently requires admission to an intensive care unit. Dutcher, et al. (2014) J Immunother Cancer 2 (1): 26. This limits the use of HD-IL2 therapy to mostly younger, very healthy patients with normal cardiac and pulmonary function.

Additionally, high doses of engineered cell therapy agents are associated with life threating cytokine release syndrome (CRS). Currently available products have shown CRS of all grades in the majority of subjects treated and Grade 3 or greater CRS in a significant fraction of patients. Significant neurotoxicity is also observed in a majority of patients. However, lower doses of the cell therapy agents have been associated with a significant decrease in clinical outcome. Additionally, due primarily to lack of persistence of the cell therapy product, many patients who at first appear be responding well to the cell therapy relapse. Currently, it is reported that approximately 60% of patients treated with existing CD-19 CAR-T cell therapy agents relapse. Byrne M, et al (2019) Biology of Blood and Marrow Transplantation 25 (11): 344-251.

Sockolosky, et al. (Science (2018) 359:1037-1042) and Garcia, et al. (United States Patent Application Publication US2018/0228841A1 published Aug. 16, 2018) describe an orthogonal IL2/CD122 ligand/receptor system to facilitate selective stimulation of cells engineered to express an orthogonal receptor, especially an orthogonal CD122. Briefly, which has been specifically modified to bind to and be activated by a variant IL2 molecule term. The contact of engineered T cells that express the orthogonal CD122 with a corresponding orthogonal ligand cognate for such orthogonal CD122 (“ortho IL2”) facilitates specific activation of such engineered T cells that express the orthogonal CD122. In particular this orthogonal IL2 receptor ligand complex provides for selective expansion of cells engineered to express the orthogonal receptor in a mixed population of cells, in particular a mixed population of T cells. The present patent application incorporates by reference the disclosures of WO 2019/104092 and US 2018-0228842 A1) in their entireties.

### BRIEF SUMMARY OF THE INVENTION

The present disclosure provides GPC3 CAR-T cells employing a selective regulation system that demonstrate efficacy in the treatment of solid tumors, particularly solid tumors expressing GPC3 (GPC3+ tumors), enable the selective expansion of GPC3 CAR-T cells in vivo, particularly in a subject undergoing treatment, demonstrate extended persistence, intratumoral infiltration of solid tumors, and the ability to treat relapse without the administration of additional GPC3 CAR-T cells.

The present disclosure relates to engineered T cells which express (a) a chimeric antigen receptor wherein the antigen binding domain of the CAR binds to human GPC3 (“a GPC-CAR”); and (b) an orthogonal receptor. The terms “chimeric antigen receptor T-cell” and “CAR-T cell” are used interchangeably to refer to a T-cell that has been recombinantly modified to express a chimeric antigen receptor. As used herein, a CAR-T cell may be engineered to express orthogonal receptor (“orthogonal CAR-T cells” or “ortho CAR-T cells”).

Also provided is a cell product substantially enriched for a population of orthogonal CAR-T cells, the product obtained by a process comprising the steps of:
a) Isolating a quantity of immune cells from a mammalian (e.g., human) subject;
(b) Contacting said isolated quantity of isolated immune cells with a nucleic acid sequence under conditions for the uptake of said nucleic acid sequence by the isolated immune cells, said nucleic acid sequence encoding an orthogonal GPC3 CAR and an orthogonal hCD122 or a functional fragment thereof;
(c) Contacting the isolated quantity of cells from step (b) ex vivo with a quantity of a orthogonal ligand sufficient to induce proliferation of cells transduced by the contacting of step (b), said contacting (c) being applied for a period of time such that the transduced cells comprise at least 20% of the cells of the population.

The present disclosure provides a mammalian immune cell comprising (a) a nucleic acid sequence encoding an orthogonal hCD122 receptor operably linked to one or more expression control elements such that the mammalian immune cell expresses the orthogonal hCD122 receptor, and (b) a nucleic acid sequence encoding a GPC3 CAR operably linked to one or more expression control elements such that the mammalian immune cell expresses the CAR. In some embodiments, the nucleic acid sequence encoding the GPC3 CAR and the nucleic acid sequence encoding the orthogonal receptor are provided on separate vectors, each nucleic acid sequence operably linked to an expression control sequence operable in a mammalian immune cell.

In some embodiments, the nucleic acid sequence encoding the GPC3 CAR and the nucleic acid sequence encoding the orthogonal receptor are provided on a single vector. In some embodiments, the nucleic acid sequences are operably linked to the same expression control element. In some embodiments, the vector comprises the two nucleic acid sequences are separated by an IRES element of T2A coding sequence. In some embodiments, the vector is a viral vector. In some embodiments, the vector is a lentiviral vector or retroviral vector.

In some embodiments, a method of treating or preventing a disease, disorder, or condition in a mammalian subject in need of treatment or prevention is provided, the method comprising the steps of:
(a) Isolating a quantity of immune cells from the subject;
(b) Contacting said isolated quantity of isolated immune cells with a nucleic acid sequence under conditions for the uptake of said nucleic acid sequence by the isolated immune cells, said nucleic acid sequence encoding a transmembrane receptor, said transmembrane receptor comprising an intracellular signaling domain in operable communication with an extracellular domain, said extracellular domain of said receptor comprising the ECD of an orthogonal hCD122 or a functional fragment thereof;
(c) Contacting the isolated quantity of cells from step (b) ex vivo with a quantity of a orthogonal ligand sufficient to induce proliferation of cells transduced by the contacting of step (b), said contacting (c) being applied for a period of time to such that the transduced cells comprise at least 20% of the cells of the population; and
(d) Administering a therapeutically effective quantity of the cells of the cell population produced from step (c) to the mammalian subject in combination with the administration of a therapeutically effective dose of an orthogonal ligand.

In some embodiments, after step (a) but prior to step (b), the population of cells is manipulated ex vivo to enrich said population for activated immune cells or antigen experienced T cells.

In some embodiments, the present disclosure provides orthogonal GPC3 CAR T cells that are recombinantly modified to express an orthogonal receptor (orthogonal immune cells). In some embodiments, the orthogonal IL2 receptor is a variant human CD122 comprising amino acid substitutions at positions 133 and 134 numbered in accordance with the wild type human CD122 (SEQ ID NO:4). In some embodiments, the orthogonal IL2 receptor is a variant human CD122 having amino acid substitutions at positions 133 and 134 wherein the substitution at position 133 is selected from the group consisting of H133D, H133E and H133F and the substitution at position Y134 is selected from the group consisting of Y143F, Y134 or Y134R numbered in accordance with SEQ ID NO.1. In some embodiments, the orthogonal IL2 receptor is a variant human CD122 having amino acid substitutions H133D and Y134F numbered in accordance with the SEQ ID NO:1 (SEQ ID NO: 2)

In some embodiments, the present disclosure provides a method of preparing an engineered immune cell product substantially enriched for orthogonal GPC3 CAR T cells the method comprising the steps of: (a) isolating a mixed population of immune cells from a subject; (b) transfecting a fraction of the population of said isolated immune cells with a recombinant vector capable of effecting the expression of an orthogonal GPC3 CAR T cells in the transfected cells; (c) culturing said mixed immune cell population in the presence of an orthogonal IL2 such that the cells expressing the orthogonal receptor selectively proliferate enriching the population of cells for cells expressing the orthogonal receptor. In some embodiments, the present disclosure provides methods for the preparation of a population of cells enriched for orthogonal GPC3 CAR T cells. In some embodiments, the present disclosure provides a population of mammalian cells enriched for orthogonal GPC3 CAR T cells.

In some embodiments, the present disclosure provides orthogonal IL2s that specifically and selectively binds to the extracellular domain (ECD) of a transmembrane polypeptide comprising of a modified CD122 polypeptide (orthogonal CD122). The binding of the orthogonal IL2 to the orthogonal CD122 participates in the transduction pathway of intracellular signaling resulting in the activation of native intracellular signaling patterns associated with IL2 binding to either the intermediate or high affinity IL2 receptor but which exhibits selectivity to an engineered cell expressing an orthogonal CD122.

The present disclosure further provides a method of extending of an active form (“persistence”) of an orthogonal GPC3 CAR T cells in vivo in a mammalian subject the administration to the subject of an effective amount of an orthogonal IL2.

The present disclosure further provides a method a method of specifically and selectively activating and/or inducing the proliferation of an orthogonal GPC3 CAR T cells in vivo in a mammalian subject by administering to the mammalian subject an effective amount of orthogonal GPC3 CAR T cells in combination with an effective amount of an orthogonal IL2.

The present disclosure further provides a method of treating a mammalian subject suffering from neoplastic disease by administering to the mammalian subject an effective amount of an orthogonal GPC3 CAR T cells in combination with the administration of a therapeutically effective amount of an orthogonal IL2, wherein the orthogonal receptor is expressed on the orthogonal CAR-T cell.

The present disclosure further provides a method of treating a mammalian subject suffering from neoplastic disease characterized by the presence of a solid tumor (e.g. HCC) by administering to the mammalian subject an therapeutically effective amount of orthogonal GPC3 CAR T cells in combination with the administration of an effective amount of an cognate orthogonal ligand for the receptor expressed on the orthogonal T cell.

The present disclosure further provides a method of restoring the activity of an exhausted therapeutically effective amount of orthogonal IL2 in a subject by the administration therapeutically effective amount of orthogonal IL2 to the subject.

The present disclosure further provides a method of treating a mammalian subject suffering from relapse of a neoplastic disease in a treatment regimen characterized by the prior administration of therapeutically effective amount of orthogonal, the method comprising the steps of: (i) administering to the subject an effective amount of orthogonal IL2 sufficient to restore the activity of the previously administered orthogonal CAR-T cells; and optionally (ii) periodically administering to the subject an effective amount of a cognate orthogonal ligand for the orthogonal receptor expressed on the orthogonal CAR-T cell previously administered to maintain the activity of orthogonal CAR-T cells for a period of time sufficient; (iii) evaluating the subject for the presence of the neoplastic disease and upon the lack of evidence of neoplastic disease either discontinuing the administration of the orthogonal ligand or continuing to administer the orthogonal ligand periodically in accordance with a maintenance dosing protocol sufficient to maintain a quantity of orthogonal CAR-T cells sufficient for immune surveillance of the neoplastic cells.

The present disclosure further provides a method of treating a mammalian subject suffering from a relapsed or refractory neoplastic disease in a treatment regimen characterized by the prior administration of orthogonal CAR-T cell product, the method comprising the steps: (i) of administering to the subject an effective amount of a cognate orthogonal ligand for the orthogonal receptor expressed on the orthogonal CAR-T cell previously administered sufficient to restore the activity of the previously administered of orthogonal CAR-T cells; (ii) periodically administering to the subject an effective amount of a cognate orthogonal ligand for the receptor expressed on the orthogonal CAR-T cell previously administered to maintain the activity of orthogonal CAR-T cells for a period of time sufficient to effect a therapeutic response; (iii) evaluating the subject for the presence of the neoplastic disease and upon the lack of evidence of neoplastic disease either discontinuing the administration of the orthogonal ligand or continuing to administer the orthogonal ligand periodically in accordance with a maintenance dosing protocol sufficient to maintain a quantity of orthogonal CAR-T cells sufficient for immune surveillance of the neoplastic cells.

In some embodiments, the GPC3 CAR is a polypeptide having at least 90%, alternatively at least 91%, alternatively at least 92%,, alternatively at least 93%, alternatively at least 94%, alternatively at least 95%, alternatively at least 96%, alternatively at least 97%, alternatively at least 98%, or alternatively at least 99% sequence identity to any one of SEQ ID NOS: 34, 35, 36, 37, 38, 39 and 40. In some embodiments, the GPC3 CAR is selected from the group consisting of SEQ ID NOS: 34, 35, 36, 37, 38, 39 and 40. In In a another embodiment as exemplified herein the GPC3 CAR is selected from the group consisting of SEQ ID NO: 37 (also referred to herein as DR625), SEQ ID NO: 38 (also referred to herein as DR626) and SEQ ID NO: 39 (also referred to herein as DR628).

In one embodiment, the disclosure provides an ortho GPC3 CAR T cell wherein the expresses on the surface of the ortho GPC3 CAR T cell: (a) a GPC3 CAR having at least 90%, alternatively at least 91%, alternatively at least 92%,, alternatively at least 93%, alternatively at least 94%, alternatively at least 95%, alternatively at least 96%, alternatively at least 97%, alternatively at least 98%, or alternatively at least 99% sequence identity to having the amino acid sequence of SEQ ID NO: 37; and (b) an ortho CD122 having at least 90%, alternatively at least 91%, alternatively at least 92%,, alternatively at least 93%, alternatively at least 94%, alternatively at least 95%, alternatively at least 96%, alternatively at least 97%, alternatively at least 98%, or alternatively at least 99% sequence identity to the amino acid sequence of SEQ ID NO:4.

In one embodiment, the disclosure provides an ortho GPC3 CAR T cell wherein the expresses on the surface of the ortho GPC3 CAR T cell: (a) a GPC3 CAR having the amino acid sequence of SEQ ID NO: 37; and (b) an ortho CD122 having the amino acid sequence of SEQ ID NO:4.

In one embodiment, the disclosure provides a method of making an ortho GPC3 CAR T cell, the method comprising the steps of:
a) obtaining a sample of human peripheral blood mononuclear cells (PBMCs);
b) contacting the sample of PBMCs by with magnetic beads coated with CD3 and CD28 ligands to provide a population of activated PBMCs
c) isolating from the a activated PBMCs cell population CD8+ and CD4+ T cells;
d) contacting the isolated population CD8+ and CD4+ T cells with a recombinant lentiviral vector, the lentiviral vector comprising an expression cassette consisting of a nucleic acid sequence encoding from 5′ to 3′:
   a promoter active in a T cell e.g. the EF1a promoter;
   a nucleic acid sequence encoding a signal peptide (
   a nucleic acid sequence encoding a GPC3 CAR;
   a T2A sequence (SEQ ID NO:41)
   a nucleic acid sequence encoding a signal peptide
   a nucleic acid sequence encoding the ortho CD122 of SEQ ID NO:4 such that a fraction of the isolated population CD8+ and CD4+ T cells is transduced with the lentiviral vector; and
e) contacting the population of cells obtained from step (d) with an ortho IL2. In a preferred practice of the foregoing method, the ortho IL2 selected from the group consisting of: (a) a human IL2 mutein containing the of amino acid substitutions E15S/H16Q/L19V/D20L/Q22K/M23A; an ortho IL2 of SEQ ID NO 9 (STK-007), or pegylated variant thereof (e.g., STK-009).

In some embodiments, the present disclosure provides an ortho GPC3 CAR T cell, the ortho GPC3 CAR T cell prepared by a method comprising the steps of:
a) obtaining a sample of human peripheral blood mononuclear cells (PBMCs);
b) contacting the sample of PBMCs by with magnetic beads coated with CD3 and CD28 ligands to provide a population of activated PBMCs
c) isolating from the an activated PBMCs cell population CD8+ and CD4+ T cells;
d) contacting the isolated population CD8+ and CD4+ T cells with a recombinant lentiviral vector, the lentiviral vector comprising an expression cassette consisting of a nucleic acid sequence encoding from 5′ to 3′:
   a promoter active in a T cell e.g. the EF1a promoter;
   a nucleic acid sequence encoding a signal peptide (
   a nucleic acid sequence encoding a GPC3 CAR;
   a T2A sequence (SEQ ID NO:41)
   a nucleic acid sequence encoding a signal peptide
   a nucleic acid sequence encoding the ortho CD122 of SEQ ID NO:4
such that a fraction of the isolated population CD8+ and CD4+ T cells is transduced with the lentiviral vector;
e) contacting the population of cells obtained from step (d) with an ortho IL2, the ortho IL2 selected from the group consisting of a human IL2 mutein containing the of amino acid substitutions E15S/H16Q/L19V/D20L/Q22K/M23A; an ortho IL2 of SEQ ID NO 9 (STK-007), or pegylated variant thereof (e.g., STK-009).

In some embodiments, the present disclosure provides a method of treating or preventing a neoplastic disease, disorder, or condition in a mammalian subject in need of treatment or prevention, the method comprising administering to said subject a therapeutically effective amount of the ortho GPC3 CAR T prepared in substantial accordance with the foregoing method in combination with a therapeutically effective dose of ortho IL. In some embodiments of the foregoing method, the ortho GPC3 CAR T administered at a dose of 4×10⁵ CAR T cells/kg. In some embodiments of the foregoing method when the ortho IL2 is STK-009, a therapeutically effective amount of STK-009 for a human subject in combination with an ortho GPC3 CAR T cell is from about 1.5 mg to about 12 mg administered subcutaneously weekly.

In some embodiments, the present disclosure provides a method of treating or preventing a neoplastic disease, disorder, or condition in a mammalian subject in need of treatment or prevention, the method consisting of the steps of:
(1) treating the subject with a lymphodepleting regimen
(2) administering to said subject a therapeutically effective amount of the ortho GPC3 CAR T prepared in substantial accordance with the foregoing method in combination with a therapeutically effective dose of an ortho IL2.

In some embodiments, the lymphodepleting regimen comprises the administration of cyclophosphamide and fludarabine. In some embodiment, the lymphodepleting regimen comprises the administration of the subject of cyclophosphamide 300 mg/m²/day and fludarabine 30 mg/m²/day for a period of three days. In some embodiments of the foregoing method, following lymphodepletion, the orthogonal GPC3 CAR T is administered at a dose of from 1×10⁵ to 5×10⁵, orthogonal GPC3 CAR T cells/kg. In some embodiments of the foregoing method, following lymphodepletion, the ortho GPC3 CAR T administered at a dose of 4×10⁵ CAR T cells/kg. In some embodiments of the foregoing method when the ortho IL2 is STK-009, a therapeutically effective amount of STK-009 for a human subject in combination with an ortho GPC3 CAR T cell is from about 1.5 mg to about 12 mg administered subcutaneously weekly.

In one embodiment of the present invention, the invention provides a method of treating a subject who has relapsed (e.g. the neoplastic disease has recurred) following the administration of ortho GPC CAR T cell therapy, the method the method comprising administering to the subject a therapeutically effective amount of an ortho IL2 such that the orthogonal ligand induces the activation and/or proliferation of the ortho GPC3 CAR T cell in the subject. In the treatment of relapse a therapeutically effective amount of STK-009 is from about 1.5 mg to about 12 mg administered subcutaneously weekly.

### BRIEF DESCRIPTION OF THE DRAWINGS

The invention is best understood from the following detailed description when read in conjunction with the accompanying figures. It is emphasized that, according to common practice, the various features of the drawings are not to-scale. On the contrary, the dimensions of the various features are arbitrarily expanded or reduced for clarity. Included in the drawings are the following figures.

FIG. 1 provides a cartoon illustration of the arrangement of the coding sequences for the various functional domains of GPC3 CARs and orthogonal CD122 components of the vectors used to produce the DR625, DR626 and DR628.

FIG. 2A-D provides a graphical presentation of data arising from studies to evaluate the efficacy in vitro cell lines of orthogonal GPC3 CAR T cells in cell lines expressing varying levels of GPC3. The vertical axis is a measure luminescence which is measurement of cell viability and the horizontal axis represents the relative dose of effector (orthogonal GPC3 CAR T cell test agent) relative to the quantity of target cells. Panel A represents the results in the PLC/PRF/5 cell line which has no or very low expression of GPC3, Panel B provides the data obtained with the Huh-7 cell line which has a low expression of GPC3 and Panel C the HepG2 cell line which has high levels of expression of GPC3. Panel D provides data illustrating comparative levels of GPC3 expression in the cell lines evaluated.

FIG. 3A-E provides a graphical presentation of data relating generated from studies to compare the effects of the orientation of the CAR and orthogonal CD122 coding sequences on the expression vector in a variety of tumor cell lines expressing high (Panel A, HepG2), intermediate/low (Panel B, PLC/PRF/5) and essentially no (Panel C, Raji) cell lines. Panels D and E are flow cytometry evaluations to assess the levels of GPC3 expression in HepG2 and PLC/PRF/5 cells respectively.

FIG. 4 provides a graphical presentation of data relating generated from studies to evaluate that the response to DR625 and DR626 ortho GPC3 CAR T cells and non-transduced cells in response to wild type human IL2 (WT IL-2) and an orthogonal IL2 (STK-009). On the vertical axis is the proliferative signal normalized to non-stimulated cells and the horizontal axis is the concentration of test agent, either wt hIL2 or STK-009.

FIG. 5A-B provides a graphical presentation of data relating generated from the HepG2 Study A relating to the in vivo evaluation of the DR625 and DR626 orthogonal GPC3 CAR T cells described. In panel A, measurement tumor volume is provided on the Y-axis and the time course of the study is provided on the X axis. The figure legend describes the treatment groups and molecules administered. Panel B provides additional data generated from HepG2 Study A relating to bodyweight changes (Y axis) in the animals over the time course of the study (X-axis).

FIG. 6 provides a series of spider plots relating to data generated with individual animals in each of the treatment groups of the HepG2 Study A. Tumor volume is provided on the Y-axis and the time course of the study is provided on the X axis with respect to each graph. Each line of the spider plot relates to an individual animal in the study.

FIG. 7 provides a series of spider plots relating to data generated with individual animals in each of the treatment groups of the HepG2 Study A. Percent change in bodyweight relative to the start of the experiment is provide on the (Y axis) the time course of the study (X-axis). Each line of the spider plot relates to an individual animal in the study.

FIG. 8 provides a graphical presentation of data relating to levels of activation (y-axis) of particular subsets of T cells (as indicated) in mice participating in the HepG2 Study A in response to treatment with the DR625 and DR626 orthogonal GPC3 CAR T cells described in combination with the orthogonal IL2 ligand, STK-009.

FIG. 9A-C provides a graphical representation of data generated in the HepG2 Study B. In panel A, tumor volume (y-axis) is provided as a function of the study duration (x-axis). In Panel B, the percent of bodyweight relative to the start of the study (y-axis) is provided as a function of the study duration (x-axis). In panel C, the number of hCD3+ cells in blood in the study animals (y-axis) is provided as a function of the study duration (x-axis).

FIG. 10 is a photomicrograph of a section of tumor obtained from animals treated from each of treatment groups HepG2 Study B stained with various fluorescent markers to highlight the presence of GPC3, CD3 and DNA in the tumor. The figure legend provides the shading associate with each fluorescent marker.

FIG. 11 is a photomicrograph of a section of tumor obtained from animals treated from each of treatment groups HepG2 Study B stained with various fluorescent markers to highlight the presence of nucleid, CD8, granzyme B and CD4 in the tumor. The figure legend provides the shading associate with each fluorescent marker.

FIG. 12 provides a graphical representation of data generated in the Huh7 Study A. In panel A, tumor volume (y-axis) is provided as a function of the study duration (x-axis). In Panel B, the percent of bodyweight relative to the start of the study (y-axis) is provided as a function of the study duration (x-axis).

FIG. 13 provides spider graphical representation of data generated in the Huh7 Study B resulting from with respect to each treatment group. Each animal is represented by a line in the graph. Tumor volume (y-axis) is provided as a function of the study duration (x-axis).

FIG. 14 provides a graphical representation of data generated in the repeat of the Huh7 study subcutaneous study. Tumor volume (y-axis) is provided as a function of the study duration (x-axis).

FIG. 15 provides a graphical representation of data generated in the repeat of the intraperitoneal Huh7 as described in the experimental section. Tumor volume (y-axis) is provided as a function of the study duration (x-axis).

FIG. 16 provides data relating to the re HepG2 Rechallenge Study A. Tumor volume (y-axis) is provided as a function of the study duration (x-axis).

FIG. 17 provides a graphical representation of data arising from the HepG2 Rechallenge Study B. Tumor volume (y-axis) is provided as a function of the study duration (x-axis).

### DETAILED DESCRIPTION OF THE INVENTION

In order for the present disclosure to be more readily understood, certain terms and phrases are defined below as well as throughout the specification. The definitions provided herein are non-limiting and should be read in view of the knowledge of one of skill in the art would know.

Before the present methods and compositions are described, it is to be understood that this disclosure is not limited to particular method or composition described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise the following abbreviation are used herein: parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=bodyweight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

The present disclosure provides a variety of IL2 muteins and CD122 receptor muteins. The following nomenclature is used herein to refer to substitutions, deletions or insertions. Residues may be designated herein by the one-letter or three-letter amino acid code of the naturally occurring amino acid found in the wild-type molecule but followed by the IL2 amino acid position of the mature IL2 molecule, e.g., “Cys125” or “C125” refers to the cysteine residue at position 125 of the wild-type hIL2 molecule. In reference to the ortho IL2s, substitutions are designated herein by the one letter amino acid code followed by the IL2 amino acid position followed by the one letter amino acid code which is substituted. For example, an ortho IL2 having the modification “K35A” refers to a substitution of the lysine (K) residue at position 35 of the wild-type IL2 sequence with an alanine (A) residue at this position. A deletion of an amino acid reside is referred to as “des” followed by the amino acid residue and its position in the mature form of wild type human IL2 (SEQ ID NO:8). For example the term “des-Alal” or “desA1” refers to the deletion of the alanine at position 1 of the polypeptide of wild-type IL2 sequence. Similarly, in reference to amino acid substitutions in the orthogonal CD122, amino acid substitutions are designated herein by the one letter amino acid code of the naturally occurring amino acid followed by the number of its position in the wild-type IL2 sequence followed by the one letter amino acid code of the amino acid which is substituted at that position. For example, the hCD122 mutein having a substitution of the tyrosine residue at position 134 with a phenylalanine residue, the substitution is abbreviated “Y134F.”

### Definitions

Unless otherwise indicated, the following terms are intended to have the meaning set forth below. Other terms are defined elsewhere throughout the specification.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand. For example, it is said that the binding of an IL2 agonist (an IL2 agonist ligand) to the IL2 receptor “activates” the signaling of the receptor to produce one or more intracellular biological effects (e.g. phosphorylation of STAT5).

Activity: As used herein, the term “activity” is used with respect to a molecule to describe a property of the molecule with respect to a test system (e.g. an assay) or biological or chemical property (e.g. the degree of binding of the molecule to another molecule) or of a physical property of a material or cell (e.g. modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. As used herein, the term “proliferative activity” refers to an activity that promotes cell proliferation and replication, including dysregulated cell division such as that observed in neoplastic diseases, inflammatory diseases, fibrosis, dysplasia, cell transformation, metastasis, and angiogenesis.

Affinity: As used herein the term “affinity” refers to the degree of specific binding of a first molecule (e.g. a ligand) to a second molecule (e.g. a receptor) and is measured by the binding kinetics expressed as K_{d}, a ratio of the dissociation constant between the molecule and the its target (K_{off}) and the association constant between the molecule and its target (Kₒₙ).

Agonist: As used herein, the term “agonist” refers an first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest or cell death such as by apoptosis. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to a substantially full biological response (i.e. the response associated with the naturally occurring ligand/receptor binding interaction) induced by receptor under study, or a partial agonist. In contrast to agonists, antagonists may specifically bind to a receptor but do not result in the signal cascade typically initiated by the receptor and may modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay. It should be noted that the biological effects associated with the full agonist may differ in degree and/or in kind from those biological effects of partial or superagonists.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway including an immune checkpoint pathway, or cell.

Antibody: As used herein, the term “antibody” refers collectively to: (a) glycosylated and non-glycosylated the immunoglobulins (including but not limited to mammalian immunoglobulin classes IgG1, IgG2, IgG3 and IgG4) that specifically binds to target molecule and (b) immunoglobulin derivatives including but not limited to IgG (1-4) deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any mammalian species and includes murine, human, equine, camelids, antibodies, human antibodies. The term antibody includes so called “heavy chain antibodies” or “VHHs” or “Nanobodies®” as typically obtained from immunization of camelids (including camels, llamas and alpacas (see, e.g. Hamers-Casterman, et al. (1993) Nature 363:446-448). Antibodies having a given specificity may also be derived from non-mammalian sources such as VHHs obtained from immunization of cartilaginous fishes including, but not limited to, sharks. The term “antibody” encompasses antibodies isolatable from natural sources or from animals following immunization with an antigen and as well as engineered antibodies including monoclonal antibodies, bispecific antibodies, tri-specific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term antibody includes both the parent antibody and its derivatives such as affinity matured, veneered, CDR grafted, humanized, camelized (in the case of VHHs), or binding molecules comprising binding domains of antibodies (e.g. CDRs) in non-immunoglobulin scaffolds. The term “antibody” should not be construed as limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries. In one embodiment, an “antibody” is a mammalian immunoglobulin. In some embodiments, the antibody is a “full length antibody” comprising variable and constant domains providing binding and effector functions. In most instances, a full-length antibody comprises two light chains and two heavy chains, each light chain comprising a variable region and a constant region. In some embodiments the term “full length antibody” is used to refer to conventional IgG immunoglobulin structures comprising two light chains and two heavy chains, each light chain comprising a variable region and a constant region providing binding and effector functions. The term antibody includes antibody conjugates comprising modifications to prolong duration of action such as fusion proteins (e.g., Fc fusions) or conjugation to polymers (e.g. polyethylene glycol) as described in more detail below.

CDRs. As used herein, the term “CDR” or “complementarity determining region” refers to the non-contiguous antigen combining sites found within the variable region of both heavy and light chain immunoglobulin polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. In the context of the present disclosure, the numbering of the CDR positions is provided according to Kabat numbering conventions.

Circulating Tumor Cell: As used herein the term “circulating tumor cell (CTC)” refers to tumor cells that have been shed from a tumor mass (e.g., neoplasm) into the peripheral circulation.

Comparable: As used herein, the term “comparable” is used to describe the degree of difference in two measurements of an evaluable quantitative or qualitative parameter. For example, where a first measurement of an evaluable quantitative parameter (e.g. a level of IL2 activity as determined by a CTLL-2 proliferation or phospho-STAT5 assay) and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 35%, alternatively by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Derived From: As used herein in the term “derived from”, in the context of an amino acid sequence (e.g., a polypeptide comprising an amino acid sequence “derived from” an IL2 polypeptide or polynucleotide sequence), is meant to indicate that the polypeptide or nucleic acid has a sequence that is based on that of a reference polypeptide or nucleic acid (e.g., a naturally occurring IL2 polypeptide or an IL2-encoding nucleic acid) and is not meant to be limiting as to the source or method in which the protein or nucleic acid is made. By way of example, the term “derived from” includes homologs or variants of reference amino acid or DNA sequences.

Effective Concentration (EC): As used herein, the terms “effective concentration” or its abbreviation “EC” are used interchangeably to refer to the concentration of an agent (e.g., an ortho IL2) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect.

Enriched: As used herein in the term “enriched” refers to a sample that is non-naturally manipulated so that a species (e.g. a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., as in a recombinantly modified bacterial or mammalian cell). In some embodiments, the term “enriched” is used herein in reference to a population of cells comprising cells that express an orthogonal receptor following contacting the population of cells with cognate ligand in an amount sufficient to cause a response in those cells that express an orthogonal receptor, the response being proliferation, such that concentration of cells that express the orthogonal receptor in the population is greater (e.g., at least 3-fold greater, alternatively at least 5-fold greater, alternatively at least 10-fold greater, alternatively at least 50-fold greater, alternatively at least 100-fold greater, or alternatively at least 1000-fold greater) after contacting with the population of cells with the cognate ligand.

Extracellular Domain: As used herein the term “extracellular domain” or its abbreviation “ECD” refers to the portion of a cell surface protein (e.g. a cell surface receptor) which is outside of the plasma membrane of a cell. The ECD may include the entire extra-cytoplasmic portion of a transmembrane protein, a cell surface or membrane associated protein, a secreted protein, a cell surface targeting protein,

hCD122: As used herein the term “hCD122” refers to a naturally occurring human CD122 polypeptide including naturally occurring variants thereof. The amino acid sequence of naturally occurring mature hCD122 is provided as SEQ ID NO 4. The human CD122 (hCD122) is expressed as a 551 amino acid pre-protein, the first 26 amino acids comprising a signal sequence which is post-translationally cleaved in the mature 525 amino acid protein. Amino acids 27-240 (amino acids 1-214 of the mature protein) correspond to the extracellular domain, amino acids 241-265 (amino acids 225-239 of the mature protein) correspond to the transmembrane domain and amino acids 266-551 (amino acids 240-525 of the mature protein) correspond to the intracellular domain. As used herein, the term CD122 includes naturally occurring variants of the CD122 protein including the CD122 variants comprising the S57F and D365E substitutions (as numbered in accordance with the mature hCD122 protein). hCD122 is referenced at UniProtKB database as entry P14784. Human CD122 nucleic acid and protein sequences may be found as Genbank accession numbers NM_000878 and NP_000869 respectively.

Identity: The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

IL2: As used herein, the term “interleukin-2” or “IL2” refers to an IL2 polypeptide that possesses IL2 activity. In some embodiments, IL2 refers to mature wild-type human IL2. Mature wild-type human IL2 (hIL2) occurs as a 133 amino acid polypeptide (less the 20 N-terminal amino acids of the signal peptide of the pre-protein), as described in Fujita, et. al., PNAS USA, 80, 7437-7441 (1983). As used herein, the numbering of residues of IL2 variants is based on the wild type mature human IL2 sequence UniProt ID P60568 excluding the signal peptide which is the same as that of SEQ ID NO: 8.

IL2 Activity: The term “IL2 activity” refers to one or more the biological effects on a cell in response to contacting the cell with an effective amount of an IL2 polypeptide. As previously noted, IL2 is a pleitropic cytokine that results one or more biological effects on a variety of cell types. IL2 promotes the proliferation and expansion of activated T lymphocytes, induces proliferation and activation of naïve T cells, potentiates B cell growth, and promotes the proliferation and expansion of NK cells. One example of IL2 activity may be measured in a cell proliferation assay using CTLL-2 mouse cytotoxic T cells, see Gearing, A. J. H. and C. B. Bird (1987) in Lymphokines and Interferons, A Practical Approach. Clemens, M. J. et al. (eds): IRL Press. 295. The specific activity of recombinant human IL2 (rhIL2) is approximately 2.1×10⁴ IU/μg, which is calibrated against recombinant human IL2 WHO International Standard (NIBSC code: 86/500). In

In An Amount Sufficient Amount to Effect a Response: As used herein the phrase “in an amount sufficient to cause a response” is used in reference to the amount of a test agent sufficient to provide a detectable change in the level of an indicator measured before (e.g., a baseline level) and after the application of a test agent to a test system. In some embodiments, the test system is a cell, tissue or organism. In some embodiments, the test system is an in vitro test system such as a fluorescent assay. In some embodiments, the test system involves the measurement of a change in the level a parameter of a cell, tissue, or organism reflective of a biological function before and after the application of the test agent to the cell, tissue, or organism. In some embodiments, the indicator (e.g. concentration of phosphorylated STAT5) is reflective of biological function (e.g. activation of the IL2 receptor) of a cell evaluated in a in an assay in response to the administration of a quantity of the test agent (e.g. IL2). In some embodiments, the test system involves the measurement of a change in the level a parameter (e.g. luminescence) of a cell, tissue, or organism (e.g. a mouse injected with luminescent neoplastic cells) reflective of a biological condition (e.g. the presence of a neoplasm) before and after the application of one or more test agents (e.g. a CAR-T cell expressing an orthogonal CD122 in combination with an orthogonal IL2) to the cell, tissue, or organism (e.g. the mouse). In some embodiments, the indicator (e.g. concentration of phosphorylated STAT5) is reflective of biological function (e.g. activation of an IL2 receptor) of a cell (e.g. a T cell) evaluated in a in an assay in response to the administration of a quantity of the test agent (e.g. IL2). “An amount sufficient to effect a response” may be sufficient to be a therapeutically effective amount but “in an amount sufficient to cause a response” may be more or less than a therapeutically effective amount.

Inhibitor: As used herein the term “inhibitor” refers to a molecule that decreases, blocks, prevents, delays activation of, inactivates, desensitizes, or down-regulates, e.g., a gene, protein, ligand, receptor, or cell. An inhibitor can also be defined as a molecule that reduces, blocks, or inactivates a constitutive activity of a cell or organism.

Isolated: As used herein the term “isolated” is used in reference to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

Kabat Numbering: The term “Kabat numbering” as used herein is recognized in the art and refers to a system of numbering amino acid residues which are more variable than other amino acid residues (e.g., hypervariable) in the heavy and light chain regions of immunoglobulins (Kabat, et al., (1971) Ann. NY Acad. Sci. 190:382-93; Kabat, et al., (1991) Sequences of Proteins of Immunological Interest, Fifth Edition, U.S. Department of Health and Human Services, NIH Publication No. 91-3242). For purposes of the present disclosure, the positioning of CDRs in the variable region of an antibody follows Kabat numbering or simply, “Kabat.”

Ligand: As used herein, the term “ligand” refers to a molecule that specifically binds a receptor and causes a change in the receptor so as to effect a change in the activity of the receptor or a response in cell that expresses that receptor. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, peptide mimetics of cytokines and peptide mimetics of antibodies. The complex of a ligand and receptor is termed a “ligand-receptor complex.” A ligand may comprise one domain of a polyprotein or fusion protein (e.g., an antibody-targeted ligand fusion protein).

Metastasis: As used herein the term “metastasis” describes the spread of cancerous cells from the primary tumor to surrounding tissues and to distant organs.

Modulate: As used herein, the terms “modulate”, “modulation” and the like refer to the ability of a test agent to cause a response, either positive or negative or directly or indirectly, in a system, including a biological system or biochemical pathway. The term modulator includes both agonists (including partial agonists, full agonists and superagonists) and antagonists.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

Neoplastic Disease: As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

Numbered in accordance with IL2: The term “numbered in accordance with IL2” as used herein refers to the identification of a location of particular amino acid with reference to the position at which that amino acid normally occurs in the sequence of the mature wild type IL2 (SEQ ID NO: 8). For example, in reference to hIL2, “R81” refers to the eighty-first (numbered from the N-terminus) amino acid, arginine, that occurs in sequence of the mature wild type hIL2. It should be noted that the amino acid sequences of IL2 molecules of different mammalian species have different numbers and sequences of amino acids. Consequently, when referencing a residue in accordance with this convention it is helpful to identify the IL2 species in question.

Numbered in accordance with CD122: The term “numbered in accordance with CD122” as used herein refers to the identification of a location of particular amino acid with reference to the position at which that amino acid normally occurs in the sequence of the mature wild type CD122 molecules. In one embodiment, the CD122 molecule is mature wild type human CD122 (SEQ ID NO. 4). For example, in reference to human CD122, H133 refers to the histidine at the one-hundred thirty third (numbered from the N-terminus) amino acid of the sequence of the mature wild type hCD122.

Operably Linked: The term “operably linked” is used herein to refer to the relationship between nucleic acid sequences encoding differing functions when combined into a single nucleic acid sequence that, when introduced into a cell, provides a nucleic acid which is capable of effecting the transcription and/or translation of a particular nucleic acid sequence in a cell. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, certain genetic elements such as enhancers need not be contiguous with respect to the sequence to which they provide their effect.

Parent Polypeptide: As used herein the terms “parent polypeptide” or “parent protein” are used interchangeably to refer to naturally occurring polypeptide that is subsequently modified to generate a mutein or variant polypeptide. A parent polypeptide may be a wild-type (or native) polypeptide. Parent polypeptide may refer to the polypeptide itself or compositions that comprise the parent polypeptide (e.g. glycosylated, pegylated, fusion proteins comprising the parent polypeptide).

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments) and the like.

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

Receptor: As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. The soluble form of hCD25 is an example of a soluble receptor that specifically binds hIL2. In some embodiments, the receptor is a cell surface receptor that comprises an extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. In some embodiments, the receptor is a membrane spanning single chain polypeptide comprising ECD, TM and ICD domains wherein the ECD, TM and ICD domains are derived from the same or differing naturally occurring receptor variants. In some embodiments, the receptor may be a hoCD122 receptor. In some embodiments, the receptor is a chimeric antigen receptor (CAR).

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and is frequently abbreviated with a lower case “r” (e.g. rhIL2) to denote the method by which the protein was produced. Similarly a cell is referred to as a “recombinant cell” if the cell has been modified by the incorporation (e.g. transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. Examples of such standard protocols to assess proliferation of CD3 activated primary human T-cells include bioluminescent assay that generates a luminescent signal that is proportional to the amount of ATP present which is directly proportional to the number of cells present in culture as described in Crouch, et al. (1993) “The use of ATP bioluminescence as a measure of cell proliferation and cytotoxicity” J. Immunol. Methods 160:81-8 or a standardized commercially available assay system such as the CellTiter-Glo® 2.0 Cell Viability Assay or CellTiter-Glo® 3D Cell Viability kits commercially available from Promega Corporation, 2800 Woods Hollow Road, Madison WI 53711 as catalog numbers G9241 and G9681 respectively in substantial accordance with the instructions provided by the manufacturer. In some embodiments, the level of activation of T-cells in response to the administration of a test agent may be determined by flow cytometric methods as described as determined by the level of STAT5 phosphorylation in accordance with methods well known in the art. STAT5 phosphorylation may be measured using flow cytometric techniques as described in Horta, et al. supra., Garcia, et al., supra, or commercially available kits such as the Phospho-STAT5 (Tyr694) kit (commercially available from Perkin-Elmer/cisbio Waltham MA as Part Number 64AT5PEG) in substantial accordance with the teaching of the manufacturer. When the abbreviation EC^{ACT} used with a subscript this is provided to indicate the concentration of the test agent sufficient to produce the indicated percentage of maximal STAT5 phosphorylation in a T cell in response to the application of the test agent as measured in accordance with the test protocol. By way of illustration, the abbreviation EC₃₀^{PRO} may be used with respect to a orthogonal hIL2 to indicate the concentration associated with 30% of a maximal level of STAT5 phosphorylation in a T cell in in response with respect to such ortho hIL2 as measured with the Phospho-STAT5 (Tyr694) kit.

In some instances, there are standardized accepted measures of biological activity that have been established for a molecule. For example with respect to hIL2 potency, the standard methodology for the evaluation of hIL2 potency in international units (IU) is measured in the murine cytotoxic T cell line CTLL-2 in accordance with standardized procedures as more fully described in Wadhwa, et al. (2013) “The 2nd International standard for Interleukin-2 (IL2) Report of a collaborative study” Journal of Immunological Methods 397:1-7. It should be noted in the context of the present disclosure that the murine IL2 receptor functions differently than the human IL2 receptor, particularly with respect to need for all components of the trimeric receptor complex to provide intracellular signal transduction signaling (e.g. STAT5 phosphorylation). See, e.g. Horta, et al., (2019) “Human and murine IL2 receptors differentially respond to the human-IL2 component of immunocytokines” Oncoimmunology 8 (6): e1238538-1, e1238538-15 and Nemoto, et al. (1995) “Differences in the interleukin-2 (IL2) receptor system in human and mouse: alpha chain is require for formation of the functional mouse IL2 receptor” European J Immunology 25 (11) 3001-5.

Selective: As used herein, the term “selective” is used to refer to a property of an agent to preferentially bind to and/or activate a particular cell type. In some embodiments, the present disclosure provides IL2 variants (ortho IL2) that selectively bind to engineered CD122 ECD polypeptides such that cells expressing receptors comprising such CD122 ECD polypeptides are activated in response to the binding of such ortho IL2 to receptors comprising such cognate CD122 ECD polypeptides. In some embodiments, the disclosure provides orthogonal hIL2 that are selective in that such ortho IL2s display preferential activation of immune cells that expressing the hoCD122 receptors. Selectivity is typically assessed by activity measured in an assay characteristic of the activity induced in response to ligand/receptor binding.

Significantly Reduced Binding: As used herein, the term “exhibits significantly reduced binding” is used with respect to the affinity of the binding of a variant of a ligand (e.g. an orthogonal IL2) to a modified form of a receptor (e.g. an orthogonal CD122) relative to the binding of the variant ligand for the naturally occurring form of a receptor. In some embodiments a ligand (e.g. an orthogonal IL2) exhibits significantly reduced binding to the native form of the ligand if the orthogonal ligand binds to the native form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the naturally occurring ligand. Similarly and orthogonal receptor exhibits significantly reduced binding with respect to the native form of the ligand if the native form of the ligand binds to the orthogonal form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the naturally occurring receptor.

Specifically Binds: As used herein the term “specifically binds” refers to the degree of affinity for which one molecule binds to another. In the context of binding pairs (e.g. a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the second molecule of the binding pair (e.g. a protein, antigen, ligand, or receptor) if the equilibrium dissociation constant between antibody and to the second molecule of the binding pair is less than about 10⁻⁶M, alternatively less than about 10⁻⁸ M, alternatively less than about 10⁻¹⁰ M, alternatively less than about 10⁻¹¹ M, alternatively less than about 10⁻¹⁰ M, less than about 10⁻¹² M as determined by, e.g., Scatchard analysis (Munsen, et al. (1980) Analyt. Biochem. 107:220-239). In one embodiment where the ligand is an ortho IL2 and the receptor comprises an orthogonal CD122 ECD, the ortho IL2 specifically binds if the equilibrium dissociation constant of the ortho IL2/orthogonal CD122 ECD is greater than about 10⁻⁵M, alternatively less than about 10⁻⁶ M, alternatively less than about 10⁻⁷M, alternatively less than about 10⁻⁸M, alternatively less than about 10⁻⁹M, alternatively less than about 10⁻¹⁰ M, or alternatively less than about 10⁻¹¹ M. Specific binding may be assessed using techniques known in the art including but not limited to competition ELISA, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET) and surface plasmon resonance assays (see, e.g., Drescher et al., Methods Mol Biol 493:323-343 (2009) with instrumentation commercially available from GE Healthcare Bio-Sciences such as the Biacore 8+, Biacore S200, Biacore T200 (GE Healthcare Bio-Sciences, 100 Results Way, Marlborough MA 01752)); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays).

Substantially Pure: As used herein in the term “substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

T-cell: As used herein the term “T-cell” or “T cell” is used in its conventional sense to refer to a lymphocytes that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells.

Transmembrane Domain: The term “transmembrane domain” or “TM” refers to the domain of a membrane spanning polypeptide (e.g. a membrane spanning polypeptide such as CD122 or CD132 or a CAR) which, when the membrane spanning polypeptide is associated with a cell membrane, is which is embedded in the cell membrane and is in peptidyl linkage with the extracellular domain (ECD) and the intracellular domain (ICD) of a membrane spanning polypeptide. A transmembrane domain may be homologous (naturally associated with) or heterologous (not naturally associated with) with either or both of the extracellular and/or intracellular domains. In some embodiments the transmembrane domain is the transmembrane domain natively associated with the ECD domain of the cognate receptor from which the orthogonal receptor is derived. In some embodiments the transmembrane domain is the transmembrane domain natively associated with the ICD domain of the cognate receptor from which the orthogonal receptor is derived. In some embodiments the transmembrane domain is the transmembrane domain natively associated with the proliferation signaling domain. In some embodiments the transmembrane domain is the transmembrane domain natively associated with a different protein. Alternatively, the transmembrane domain of the orthogonal receptor may be an artificial amino acid sequence which spans the plasma membrane. In some embodiments, the transmembrane domain of the orthogonal receptor is the transmembrane domain normally associated with the ICD of the cognate receptor from which the orthogonal receptor is derived. In some embodiments, where the receptor is chimeric receptor comprising the intracellular domain derived from a first parental receptor and a second extracellular domains are derived from a second different parental receptor, the transmembrane domain of the chimeric receptor is the transmembrane domain normally associated with either the ICD or the ECD of the parent receptor from which the chimeric receptor is derived.

Treat: The terms “treat”, “treating”, treatment” and the like refer to a course of action (such as administering IL2, a CAR-T cell, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

Treg Cell or Regulatory T Cell. The terms “regulatory T cell” or “Treg cell” as used herein refers to a type of CD4⁺ T cell that can suppress the responses of other T cells including but not limited to effector T cells (Teff). Treg cells are characterized by expression of CD4, the α-subunit of the IL2 receptor (CD25), and the transcription factor forkhead box P3 (FOXP3) (Sakaguchi, Annu Rev Immunol 22, 531-62 (2004). By “conventional CD4⁺ T cells” is meant CD4⁺ T cells other than regulatory T cells.

Variant: The terms “protein variant” or “variant protein” or “variant polypeptide” are used interchangeably herein to refer to a polypeptide that differs from a parent polypeptide by virtue of at least one amino acid modification. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. In some embodiments, the variant polypeptide comprises from about one to about ten amino acid modifications relative to the parent polypeptide, alternatively from about one to about five amino acid modifications compared to the parent, alternatively from about one to about three amino acid modifications compared to the parent, alternatively from one to two amino acid modifications compared to the parent, alternatively a single amino acid modification compared to the parent. A variant may be at least about 99% identical, alternatively at least about 98% identical, alternatively at least about 97% identical, alternatively at least about 95% identical, or alternatively at least about 90% identical to the parent polypeptide from which the variant is derived.

Wild Type: By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been modified by the hand of man.

### DETAILED DESCRIPTION

### Overview

The present disclosure relates to engineered T cells which express (a) a chimeric antigen receptor wherein the antigen binding domain of the CAR binds to human GPC3 (“a GPC-CAR”); and (b) an orthogonal receptor. The terms “chimeric antigen receptor T-cell” and “CAR-T cell” are used interchangeably to refer to a T-cell that has been recombinantly modified to express a chimeric antigen receptor. As used herein, a CAR-T cell may be engineered to express orthogonal receptor (“orthogonal CAR-T cells” or “ortho CAR-T cells”).

The present invention is directed to compositions and methods relating to ortho GPC3 CAR T cells in combination with an ortho IL2 ligand. A variety of benefits flow from the use of ortho GPC3 CAR T cells in combination with an ortho IL2

In one embodiment, the present disclosure provides compositions and methods that provide selective expansion a population of adoptively transferred human ortho GPC3 CAR-T cells in a human subject upon administration of an ortho IL2 to the subject.

In another embodiment, the present disclosure provides compositions and methods that enable the selective expansion a population of adoptively transferred human orthogonal GPC3 CAR-T cells. in vivo without significant off-target systemic activation of other immune cells.

In another embodiment, the present disclosure provides compositions and methods that support the persistence of ortho GPC3 CAR-T cells adoptively transferred human immune cells the without significant toxicity associated with the alternative supportive agent such as wt hIL2.

In another embodiment, the present disclosure provides compositions and methods that achieve in vivo therapeutic effectiveness of a cell therapy product comprising ortho GPC3 CAR-T cells in the treatment of neoplastic disease in a mammalian subject using an initial dose of the cell therapy agent at doses that have previously been reported as non-efficacious and significantly below current dosages of similar cell therapy products;

In another embodiment, the present disclosure provides compositions and methods that enable the maintenance of a therapeutic level of an ortho GPC3 CAR-T cells at a therapeutically effective level in a subject for extended periods of time by periodic administration of an ortho IL2 to the subject.

In another embodiment, the present disclosure provides compositions and methods that enable the treatment of relapse of a neoplastic condition in a subject previously treated with ortho GPC3 CAR-T cells by the administration of an ortho IL2 to revive the effectiveness of the of previously administered orthogonal cells without the need to administer additional ortho GPC3 CAR-T cells.

In another embodiment, the present disclosure provides compositions and methods that avoid the need for prior immunodepletion of the subject prior to administration of a cell product comprising ortho GPC3 CAR-T cells.

In another embodiment, the present disclosure provides compositions and methods relating to pharmaceutical formulations of ortho GPC3 CAR-T cells and ortho IL2.

In another embodiment, methods of treatment of a subject suffering from a GPC3+ tumor by the administration of a therapeutically effective amount ortho GPC3 CAR-T cells in combination with a pharmaceutically acceptable formulation of an ortho IL2.

### EXPERIMENTAL

To demonstrate the efficacy of the compositions and methods encompassed within the scope of the present disclosure, a series of experiments were conducted to evaluate the anti-GPC3+ tumor efficacy of ortho GPC3 CAR T cells both in vitro and in vivo as described in detail below and the data is provided in the attached Figures.

### A. Preparation of GPC3 CAR-T Cells for Studies

Nucleic acid sequences encoding the GPC3 CARS were synthesized using conventional DNA sequence technology. The amino acid sequence of GPC3 CARs DR625, DR626 and DR628 are provided as SEQ ID NOS: 37, 38, and 39 respectively. The DNA sequences of nucleic acids encoding used in the construction of the lentiviral vectors to generate GPC3 CARs DR625, DR626 and DR628 are provided as SEQ ID NOS: 42, 43 and 45 respectively.

The basis for the lentiviral vector used for construction of the ortho GPC3 CAR T cells used in this study was a modified version of the LV200 pLenti-EF1-MCS plasmid (commercially available from Alstem, Inc. 2600 Hilltop Drive, Richmond CA USA as Catalog No. LV200). The vector was modified to insert an NheI restriction site into the MCS cloning site of the vector to facilitate insert of the GPC3 CAR sequences and a Kozak sequence was added downstream of the EcoRI site. The DNA sequence of the resulting vector is SEQ ID NO: 47.

A population of human PBMCs was obtained and the CD4/CD8 T cells isolated from the PBMCs by flow cytometry. The CD4/CD8 T cells were stimulated with CD3.CD28 beads (Miltenyi) for a period of 24 hours.

Healthy donor primary blood mononuclear cells (PBMCs) were isolated from leukopaks via Ficoll-Paque separation (Global Life Sciences Solutions). CD4 and CD8 T cells were stained with CD4 and CD8 microbeads and isolated using MACS magnetic separation columns (Miltenyi Biotec). Cells were stimulated with anti-CD3 (clone OKT3, Miltenyi Biotec) and anti-CD28 antibody (clone CD28.2, BD Biosciences).

48h post-stimulation, cells were transduced with lentivirus and maintained in complete OpTmizer T cell media (Gibco) with wild type IL-2 (Miltenyi Biotec). 24-48 hours post-transduction, cells were washed and either maintained in WT IL-2 containing media or switched to STK-009 containing media. Cells were expanded with media exchanges every other day. Cell counts and viability were performed using a Vi-Cell XR (Beckman Coulter). On day 7-14, cells were frozen down in CryoStor CS10 cell freezing medium (STEMCELL).

The cells were transduced with lentivirus encoding comprising the nucleic acid sequence encoding the GPC3-CAR and ortho CD122 coding sequences separated by a T2A sequence was added at an infectivity ratio and cultured for a period of one to three days at which time the cells were washed to remove excess lentivirus and resuspended in fresh media.

The DNA sequences encoding the DR625, DR626 and DR628 constructs were inserted into the multiple cloning site (MCS) of the LV200 pLenti-EF1-MCS plasmid (commercially available from Alstem, Inc. 2600 Hilltop Drive, Richmond CA USA as Catalog No. LV200 Alstem

The ortho IL2 molecule used in these studies is PEGylated hIL2 variant containing the amino acid deletion and substitutions desAla1/E15S-H16Q-L19V-D20L-Q22K-M23A comprising a N-terminally monopegylated 40 kD branched (2×20 kD) PEG molecule with an aldehyde linker. The 40 kDa 2-arm branched PEG-aldehyde comprising two 20 kDA linear PEG molecules was Sunbright® GL2-400AL3 (commercially available, NOF America Corporation, One North Broadway, White Plains, NY 10601 USA.

Female NOD scid gamma (NSG) mice aged 6 to 8 weeks were obtained from The Jackson Laboratory, 600 Main Street, Bar Harbor, ME USA 04609). Prior to initiation of the study, animals were weighed and given a clinical examination to ensure that they were in good health. Weights were then tracked through the duration of the study.

Except as noted below, the administration of tumor cells was administered in a 50:50 ratio of tumor cells and phenol-red free Matrigel (Corning).

For purposes of the following studies, the GPC3 CAR T cells are identified by the GPC3 CAR that they express.

### B. In Vitro Evaluation GPC3 Negative: GPC3 Low and GPC3 High Cell Lines

An initial series of experiments was performed to evaluate the effect of differing co-stimulatory domains in the GPC3 CAR T cells, a series of experiments were conducted to compare GPC3 CAR T cells containing the CD28 (DR625) and 41BB (DR628) in a variety of cell lines having varying levels of expression of GPC3 in vitro as well as evaluate the activity of

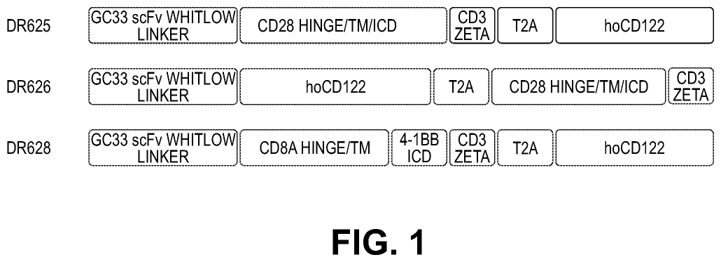

Also evaluated was the effect of orientation of the costimulatory domain and the CAR coding sequences on the vector. As illustrated in FIG. 1, in DR625, the CD28 GPC3-CAR sequence is 5′ to the ortho CD122 sequence. However, in DR626, the orientation of the CD28 GPC3-CAR and the ortho CD122 sequence are reversed such that the ortho CD122 sequence is 5′ to the CD28 GPC3-CAR sequence.

A series of experiments were conducted cells to evaluate cytotoxicity of ortho GPC3 CAR T cells both in vitro and in vivo in cell lines having varying levels of expression of GPC3. PLC/PRF/5 were obtained (ATCC under accession number CRL-8024) which is a hepatocellular carcinoma cell line that is GPC low or negative.

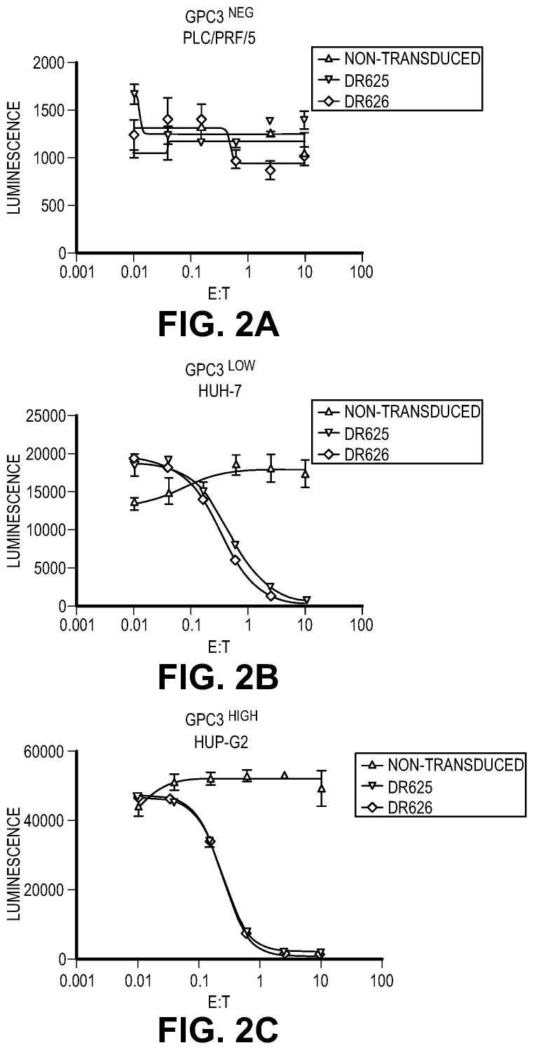

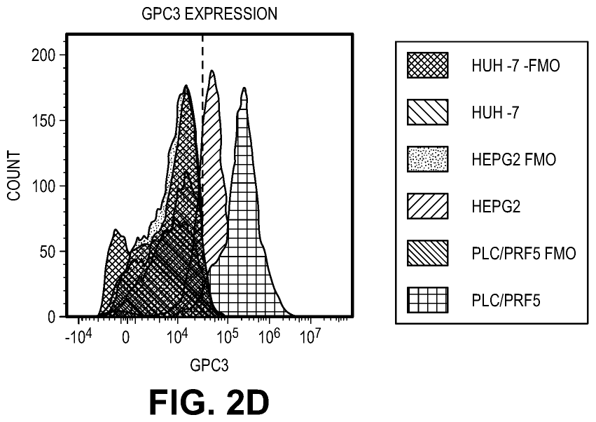

The data provided in FIG. 2 provides the results of this series of experiments. As illustrated in FIG. 2, ortho GPC3 CAR T cells cells selectively kill GPC3^{pos} cells (FIG. 2 panels B and C) but do not show significant specific cytotoxicity with respect to GPCneg (PLC/PRF/5 cells, Panel A) cells.

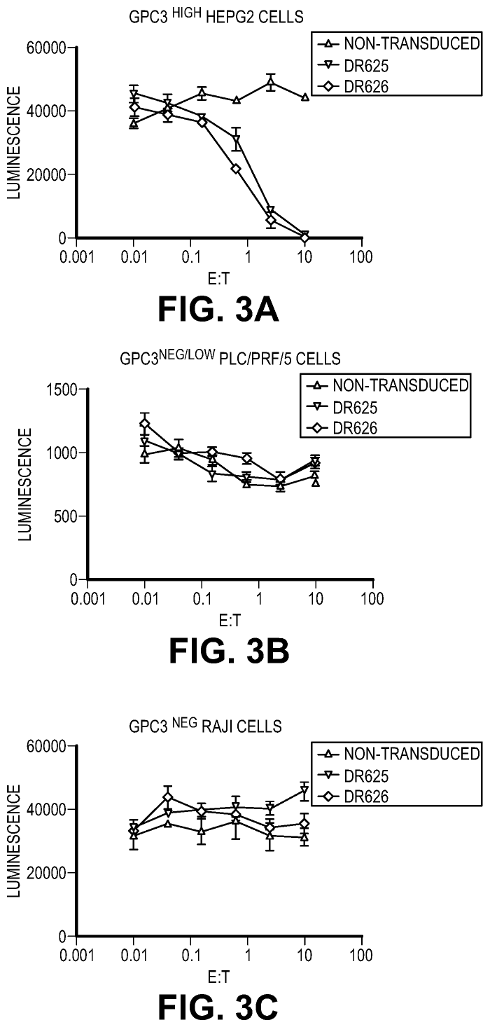

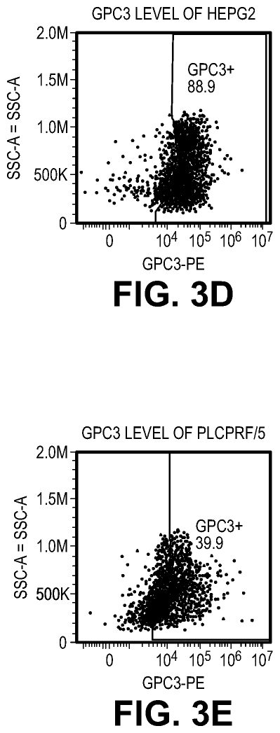

Also evaluated was the effect of orientation of the costimulatory domain and the CAR coding sequences on the expression vector. As illustrated in FIG. 1, in DR625, the CD28 GPC3-CAR sequence is 5′ to the ortho CD122 sequence. However, in DR626, the orientation of the CD28 GPC3-CAR and the ortho CD122 sequence are reversed such that the ortho CD122 sequence is 5′ to the CD28 GPC3-CAR sequence. This data is provided in FIG. 3 of the attached drawings. As illustrated, the DR625 and DR626 ortho GPC3 CAR T cells both showed selective cytotoxicity as a function of GPC3 expression level on the target cells. The data indicates that the orientation of the GPC-CAR and ortho CD122 coding sequences on the vector have little or no influence on cytotoxicity of the ortho GPC3 CAR T cells as the DR625 and DR626 ortho GPC3 CAR T cells performed similarly in this study.

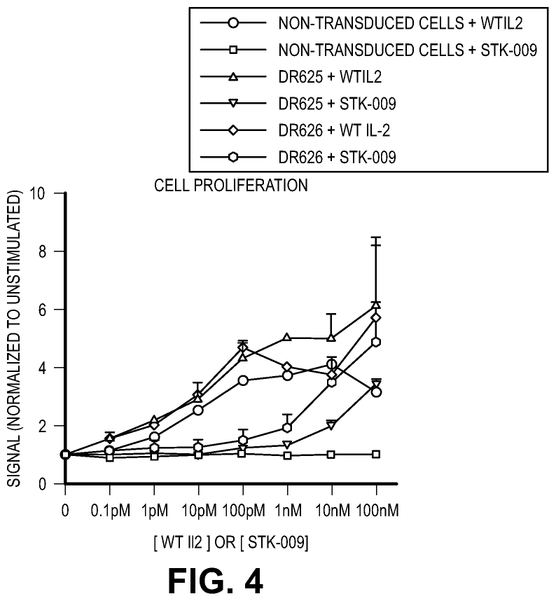

To demonstrate that the DR625 and DR626 ortho GPC3 CAR T cells are selectively responsive to exposure to an ortho IL2, a dosing study was conducted to compare to DR625 and DR626 ortho GPC3 CAR T cells and non-transduced T cells. The results of this study is provided in FIG. 4 of the attached drawings. As all cells express the wild type IL2 receptor, all cells were responsive to wild type IL2. However, the DR625 and DR626 ortho GPC3 CAR T cells were selectively expanded in the presence of the ortho IL2 ligand whereas the non-transduced cells were nonresponsive to exposure to ortho IL2. This data demonstrates that ortho GPC3 CAR T cells are selectively responsive to contact with an ortho IL2 ligand.

B. In Vivo Evaluation of Ortho GPC3 CAR T Cells in Tumors with High GPC3 Expression (HepG2)

A series of experiments were conducted to evaluate the performance of ortho GPC3 CAR-T cells in a xenograft solid epithelial tumor model of hepatocellular carcinoma using HepG2 cells. Hepatocellular carcinoma (HCC) is the third prevalent cause of cancer death worldwide. Despite recent advances in diagnosis and treatment, HCC is frequently diagnosed at an advanced stage and has a poor prognosis

1. HepG2 Study A (S2-21-001): Subcutaneous HepG2 Hepatocellular Carcinoma Model in NSG Mice with DR625 and DR626

HepG2 cells (Wistar Institute) were maintained in exponential growth phase prior to collection. The cells were collected by trypsinizing the cells and suspended in media. Cell concentration and viability were determined with trypan blue (min 98% viability). Cell suspensions were then adjusted to the required concentration for inoculation.

On day 0 of the study, approximately one million HepG2 cells in a volume of 100 μL (Matrigel+HepG2 suspension) was inoculated subcutaneously via a single injection into the hind leg of approximately 10 week old NSG™ mice (Jackson Labs). The injection areas were monitored until the tumors palpable. Calipers were used for tumor measurement and tumors were allowed to progress until average sizes of tumors are 50-150 mm³ prior to treatment. After randomization (sorting) into treatment groups in accordance with following table.

| | Ortho GPC3 | Dose of | | | |
|---|---|---|---|---|---|
| | CAR T | CAR T | Additional | Dose/ | |
| Group | Agent | Cells | Treatment | Frequency | Mice/Group |
| 1 | n/a | n/a | PBS | | 8 |
| 2 | DR626 | 2 × 10⁶ | PBS | | 8 |
| 3 | DR626 | 2 × 10⁶ | STK-009 | 10 ug q.o.d. | 8 |
| 4 | DR625 | 1 × 10⁶ | PBS | | 4 |
| 5 | DR625 | 1 × 10⁶ | STK-009 | 10 ug q.o.d. | 4 |

On day 10 of the study the CAR T cells indicated in the above table was administered intravenously to the mice. The additional treatment indicated in the above table was administered subcutaneously every other day (q.o.d.). With respect to treatment Groups 3 and 5, the STK-009 dose was interrupted due to bodyweight loss and a single additional dose of STK-009 was administered at Day 40 of the study to Group 3 and on day 33 in group 5. Tumor measurements were recorded (daily) and mouse weights were documented (3 times weekly). As tumor size limit is reached (a maximum of 2,000 mm3) the animals were euthanized.

At the conclusion of the treatment phase, the animals were sacrificed, and necropsy was performed. The tumors were removed, and tumors were weighed and the tumor was documented by digital imaging). Various tissues were collected from the sacrificed animals (submersed in RNAlater® (Sigma Aldrich), snap frozen, nucleic acids were isolated, and tissues prepared for histological analysis by performing standard gross necropsies.

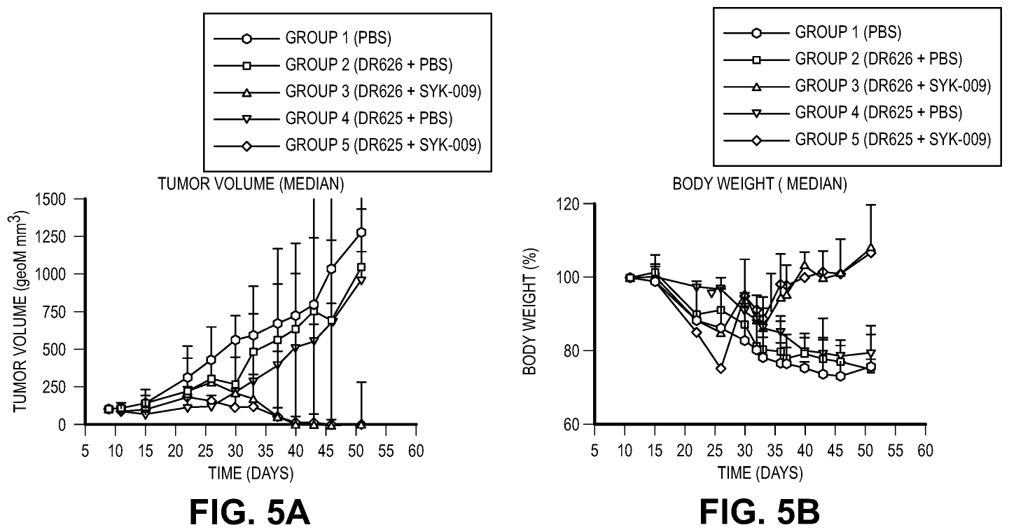

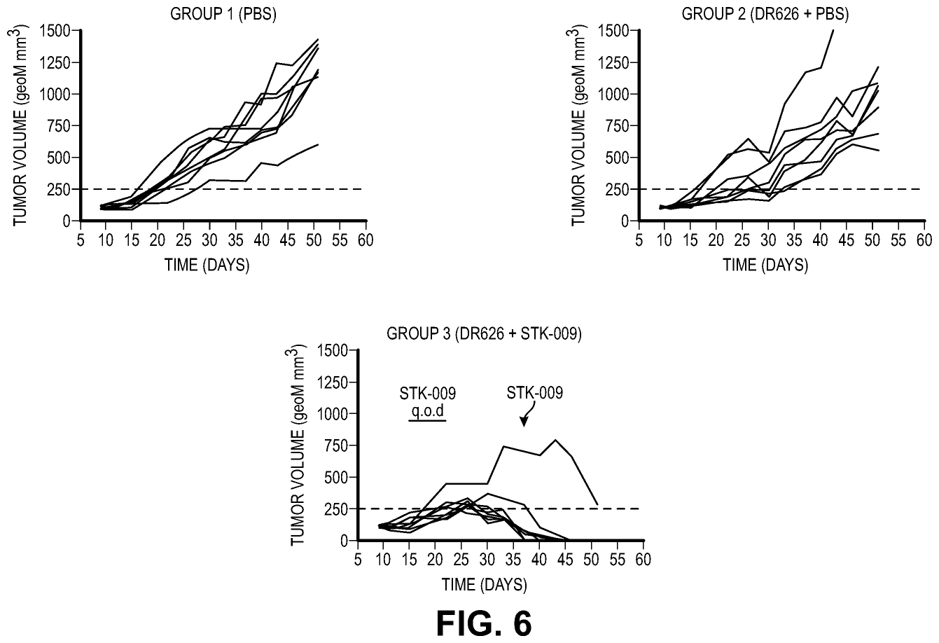

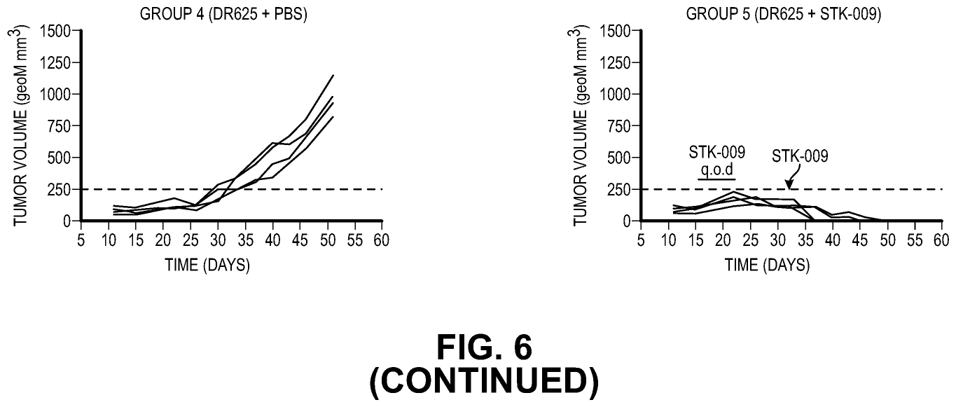

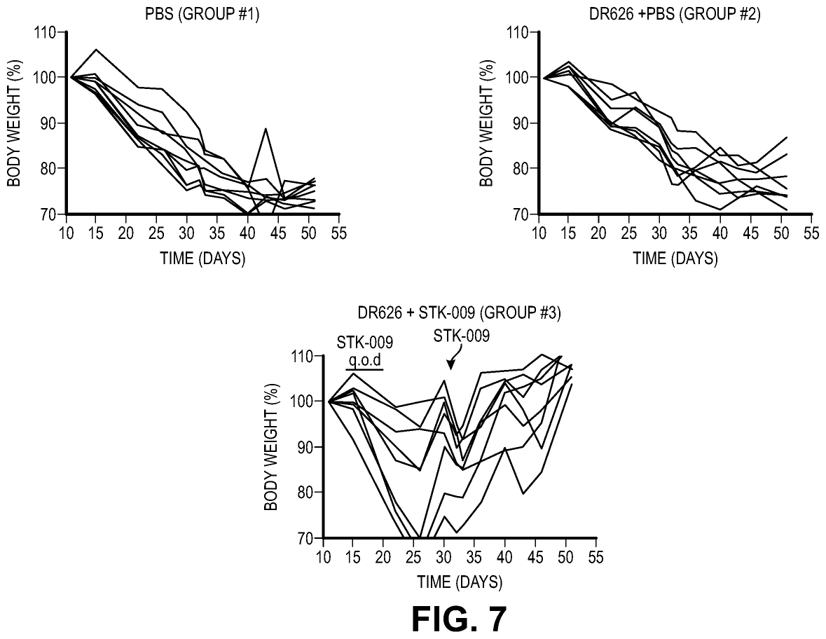

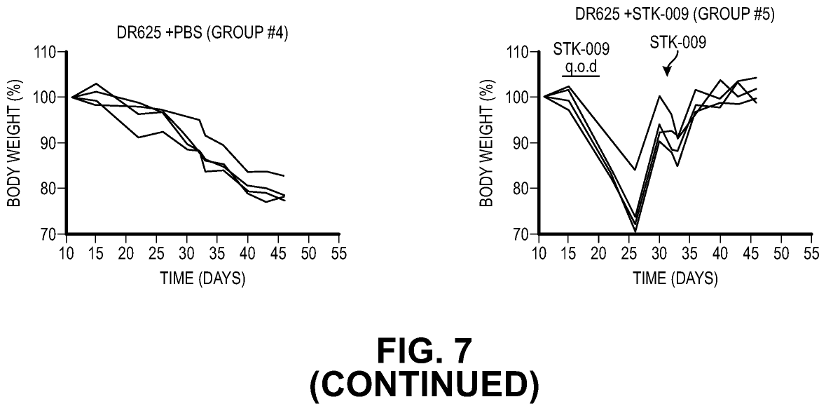

The data from this study with respect to tumor volume (efficacy) and bodyweight (toxicity) is provided in FIG. 5, Panels A and B respectively. Spider plots with respect to each treatment group of this study are provided in FIG. 6. Bodyweight spider plots are provided in FIG. 7. The tumor volume spider plots demonstrate durable complete responses in the mice treated with the DR625 ortho GPC3 CAR T cells in combination with STK-009.

Although mice treated with STK-009 and DR625 and DR626 ortho GPC3-CAR T cells exhibited a transient loss in bodyweight in this study, the animals did recover and the toxicity was reversible. Subsequent studies were performed whereby the STK-009 dose schedule and dose levels were titrated to avoid toxicity while still maintaining anti-tumor efficacy. It should be noted that the dose of DR625 and DR626 ortho GPC3-CAR T cells was particularly high (e.g., 10 μg q.o.d. (every other day)) and efficacy has been demonstrated at lower doses (e.g., 1 μg/weekly).

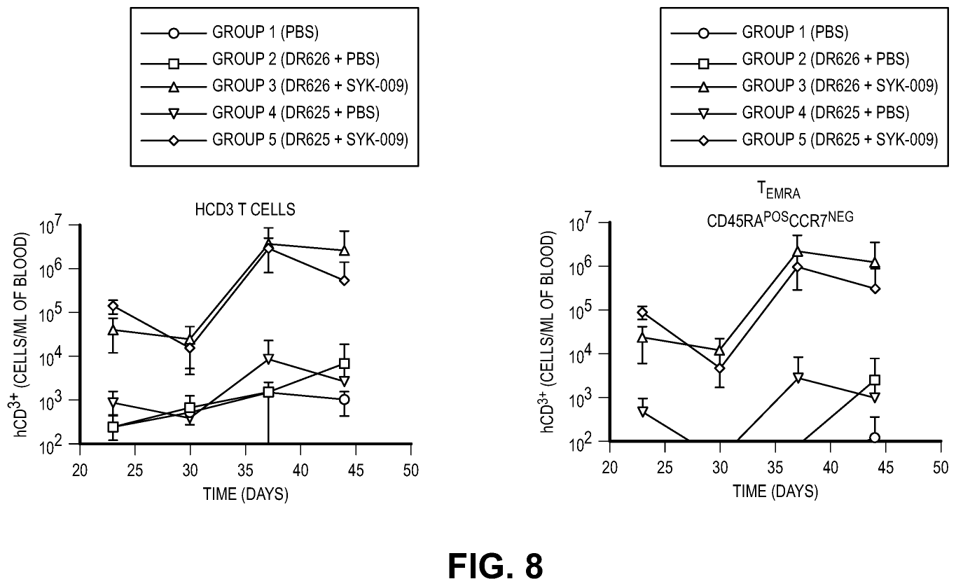

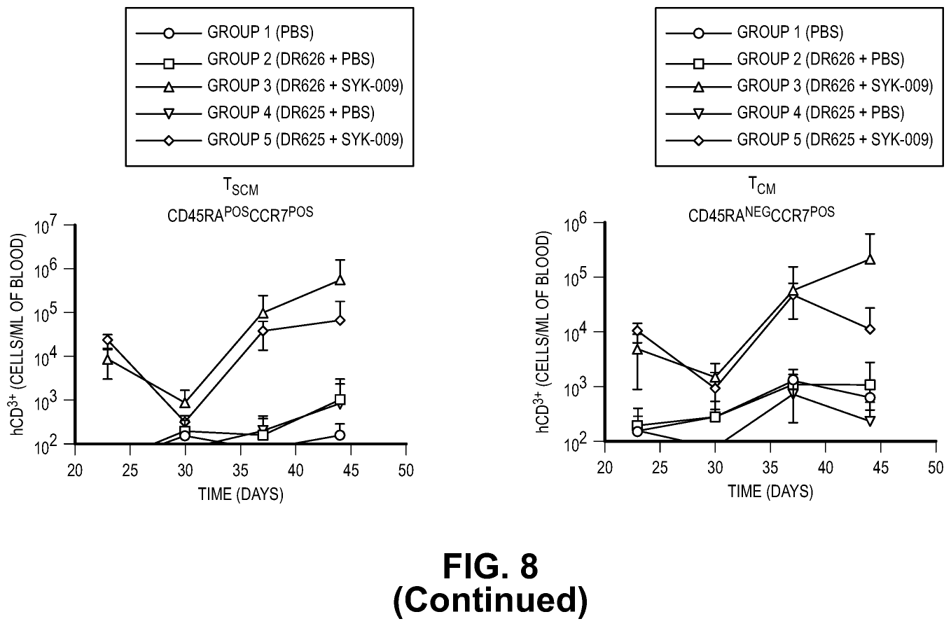

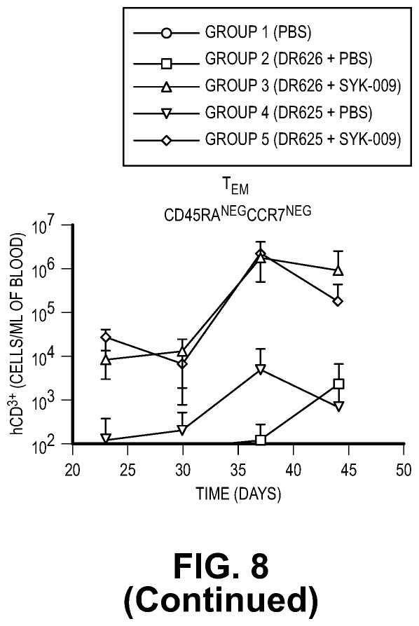

Over the course of this study, blood was sampled weekly from each treatment group and CD8+ T cells selected by FACS. Cell types were distinguished by CCR7 CD45RA status sorted by flow cytometry. Four human CD8+ T-cell subsets, naive (CCR7+CD45RA+), central memory (TCM, CCR7+CD45RA−), effector memory (TEM, CCR7−CD45RA−), and CD45RA+ effector memory cells (TEMRA, CCR7-CD45RA+) were compared for their capacity to proliferate and differentiate in response to antigen or homeostatic cytokines. Geinat, et al (2003) Proliferation and differentiation potential of human CD8+ memory T-cell subsets in response to antigen or homeostatic cytokines Blood; 101 (11): 4260-6. The data is presented in FIG. 8 of the attached drawings. As illustrated in FIG. 8, the STK-009 treatment induces significantly elevated GPC3_28z orthoCAR T cell levels across all memory subtypes.

2. HepG2 Study B (S2-21-009): Subcutaneous HepG2 Hepatocellular Carcinoma Model with DR625

An additional study to evaluate the effects of an ortho GPC3 CAR T cells in combination with the administration of an ortho IL2 (STK-009) was conducted wherein the dose of the STK-009 was provided less frequently but over a longer period of time when compared to HepG2 Study A discussed above. The study was conducted in substantial accordance with HepG2 Study A discussed above with the following modifications. In this HepG2 Study B, a single ortho GPC3 CAR T cell (DR625) was evaluated a lower dose (8.5×10⁵) cells in as compared to HepG2 Study A above (1×10⁶). In this HepG2 Study B, satellite arms were provided for additional immunohistochemical analysis. Whole slide scanning and signal quantification was performed using Akoya Vectra multi-spectral imaging system and Akoya Vectra InForm analysis software suite. As compared to the 1×10⁶ HepG2 cells administered in HepG2 Study A above 5×10⁶ HepG2G tumor cells were used in in Matrigel were administered on day 0 of the study HepG2 Study A. Treatment with ortho GPC3 CAR T cell began on day 7. The additional treatment was administered every third day beginning on Day 7 through day 43. The design of the study and various treatment groups is summarized in the following table.

| | | Ortho | | | | | | |
|---|---|---|---|---|---|---|---|---|
| | Ortho | GPC3 | | | Mice | Mice | Mice | |
| | GPC3 | CAR T | | | evaluated | evaluated | evaluated | |
| | CAR T | Cell Dose | Additional | Tumor | for | for IHC | for IHC | Mice for |
| Group | Cell | (cells) | Treatment | bearing? | Efficacy | (1/week) | (2/week) | rechallenge |
| 1 | n/a | | PBS | Yes | 5 | — | — | — |
| 2 | DR625 | 8.5 × 10⁵ | PBS | Yes | 8 | 3 | 3 | — |
| 3 | DR625 | 8.5 × 10⁵ | STK-009 | Yes | 9 | 3 | 3 | 10 |
| | | | 10 ug 2x/week | | | | | |

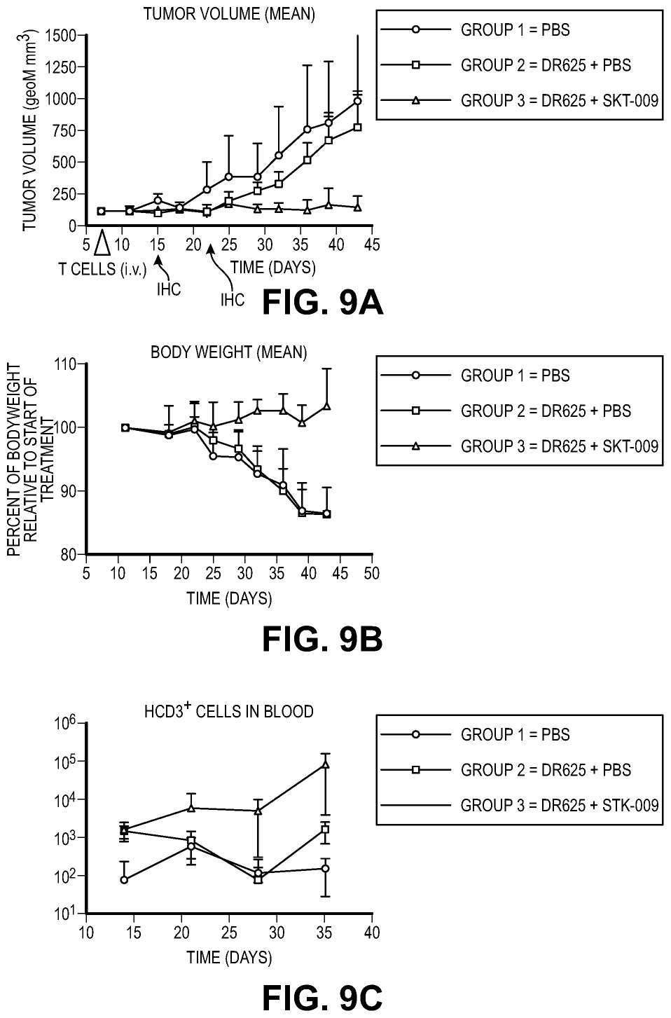

The results of this study are summarized with respect to efficacy (tumor volume) in FIG. 9A, with respect to toxicity (bodyweight) in 9B and with respect to activated T cells (CD3+ cells) in 9C.

As illustrated in FIG. 9A, the antitumor efficacy in response to the combination of ortho GPC3 CAR T cell (DR625) in combination with an ortho IL2 (STK-009) results in prolonged antitumor efficacy. It should be noted that tumors in the animals in Group 3 treated with an of ortho GPC3 CAR T cell (DR625) in combination with an ortho IL2 (STK-009) failed to grow throughout the course of the study through day 43 demonstrating persistence of efficacy of the ortho GPC3 CAR T cells when administered in combination with an ortho IL2.

As illustrated in FIG. 9B, no loss of bodyweight was observed in those animals in Group 3 which were treated with ortho GPC3 CAR T cell (DR625) in combination with an ortho IL2 (STK-009) demonstrating that ortho GPC3 CAR T cells when administered in combination with an ortho IL2 is well tolerated in a mammalian subject.

As illustrated in FIG. 9B, Group 3 animals receiving treatment with an ortho GPC3 CAR T cell (DR625) in combination with an ortho IL2 (STK-009) exhibited specific increases in hCD3+ cells in blood indicating that there is in vivo expansion of the GPC3 CAR T cells when administered in combination with an ortho IL2.

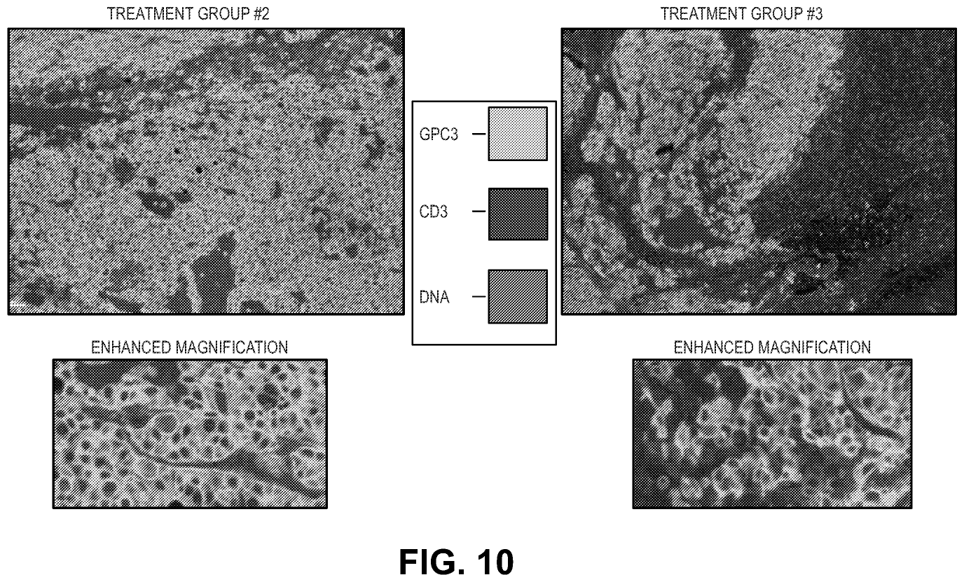

Immunohistochemical analysis was conducted in accordance with the foregoing protocol and the FIG. 10 provides the results of this analysis demonstrating intratumoral apoptosis at Day 29 of the study (22 days following initiation of treatment with ortho GPC3 CAR T cell (DR625) in combination with an ortho IL2 (STK-009). In this analysis GPC3 stained green, CD3 stained red and DNA stained blue. This data demonstrates ortho GPC3 CAR T cells when administered in combination with an ortho IL2 results in intratumoral apoptosis of tumor cells in a solid tumor.

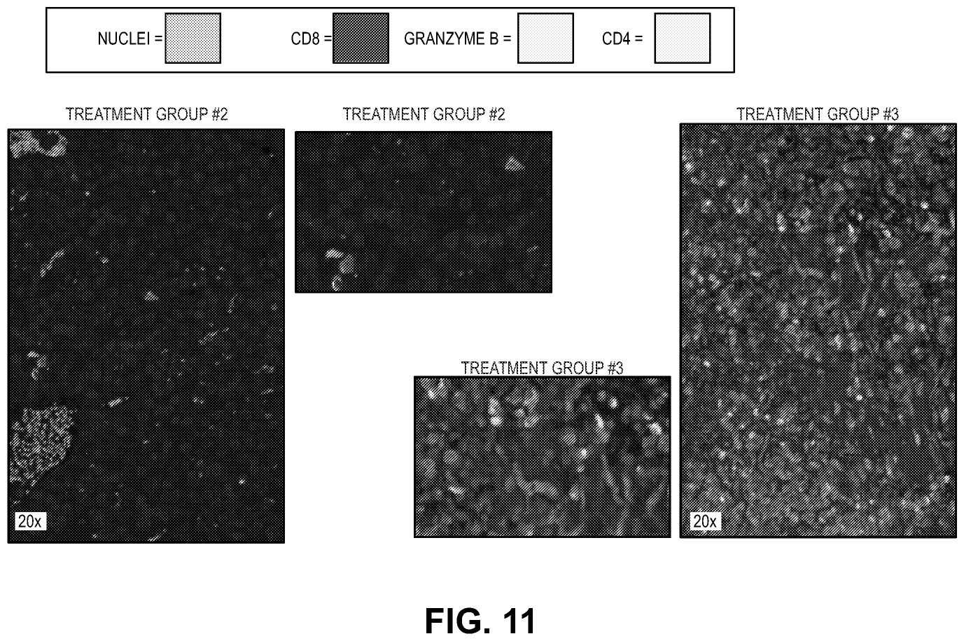

Additional immunohistochemical analysis was conducted and the results are presented in FIG. 11 of the attached drawings. The multicolor staining results are presented here and evaluated for the presence of nuclei (blue), CD8+ cells (red), granzyme B (green) and CD4 cells (yellow This data demonstrates ortho GPC3 CAR T cells when administered in combination with an ortho IL2 results in intratumoral activation of ortho GPC3 CAR T cells.

B. In Vivo Evaluation of Ortho GPC3 CAR T Cells in Tumors with Low GPC3 Expression

As series of studies was conducted to evaluate the efficacy of an ortho GPC3 CAR T cell (DR625) in combination with an ortho IL2 (STK-009) in the treatment of Huh7 tumors expressing lower levels of GPC3 relative to the foregoing studies in HepG2 tumors which express comparatively high levels of GPC3.

### 1. Huh7 Study A (S2-21-017): DR625 in an Huh7 In Vivo Model

This Huh7 Study A study was conducted in substantial accordance with the procedures described above in relation to the HepG2 Study A with certain modifications as noted below. An initial subcutaneous implant of 7.5×10⁵ Huh-7 cells failed to generate tumors after 17 days. Consequently, a second subcutaneous implant of 2.5×10⁶ cells on opposite flank which elicited the growth of tumors. The treatment groups and treatments are summarized in the following table:

| | Ortho GPC3 | Ortho GPC3 CAR | | |
|---|---|---|---|---|
| Group | CAR T Cell | T Cell Dose | Additional Treatment | Mice/Efficacy |
| 1 | n/a | n/a | PBS | 6 |
| 2 | DR625 | 2 × 10⁶ cells IV | PBS | 7 |
| 3 | DR625 | 2 × 10⁶ cells IV | STK-009 | 7 |
| | | | (10 ug SQ /2x week) | |
| | | | switched to 3x/week on 21 | |

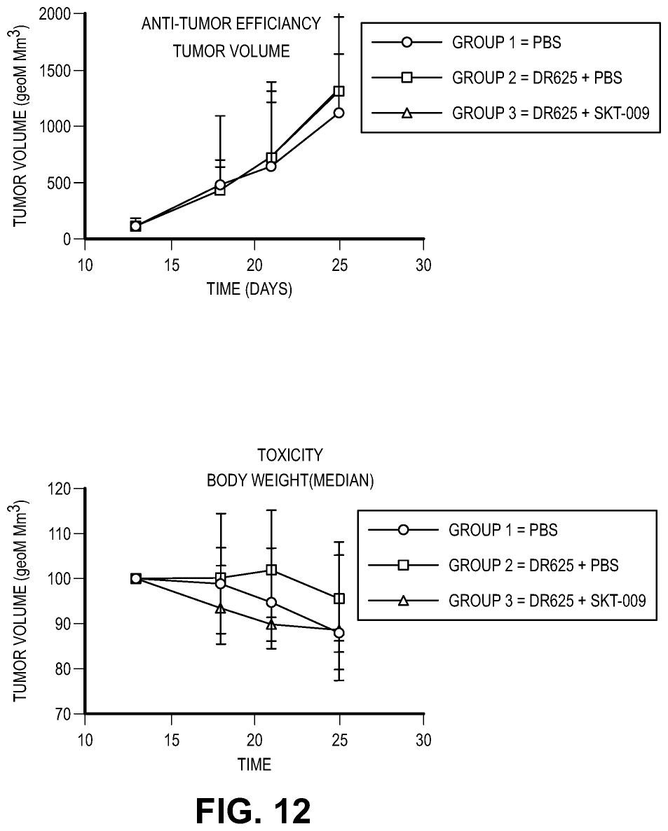

A single intravenous ortho GPC3 CAR T cell (DR625) transfer was conducted 13 days following the subcutaneous implant of 2.5×10⁶ Huh7 cells (study day 13). The administration of STK-009 began on study day 13 and continued through study day 43. The results of this study are presented in FIG. 12 of the attached drawings.

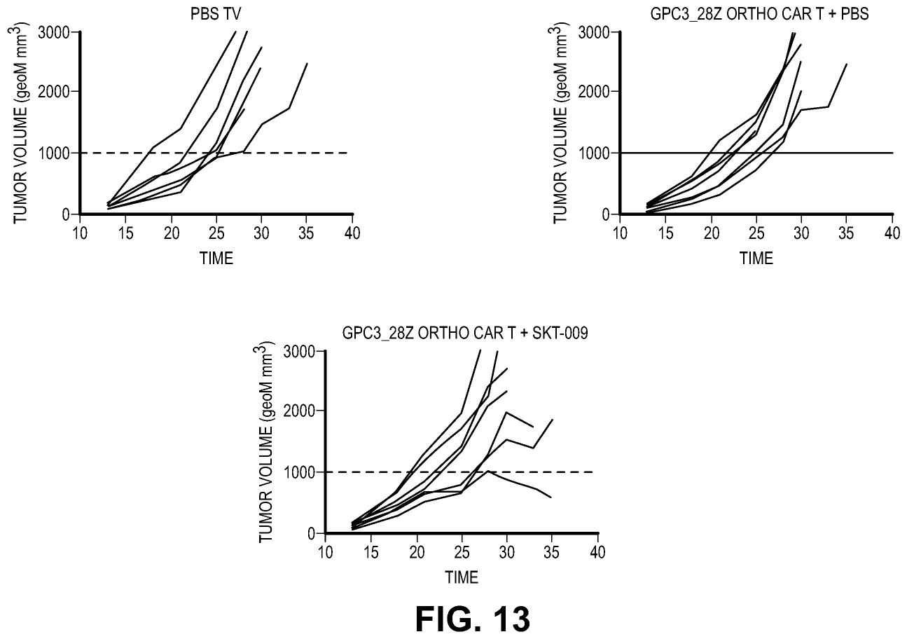

As illustrated in the data summarized on FIG. 12, the combination of the ortho GPC3 CAR T cell (DR625) in combination with an ortho IL2 (STK-009) failed to provide meaningful tumor control while toxicity was comparatively mild when evaluated in light of tumor burden. The spider plots with respect to individual animals (FIG. 13) indicate that certain animals in Group 3 responded to the treatment. In view of the difficulties in this experiment in obtaining initial tumor growth, two administrations of tumor cells intraperitoneally and subcutaneously, this study was repeated as described in Huh7 Study B below.

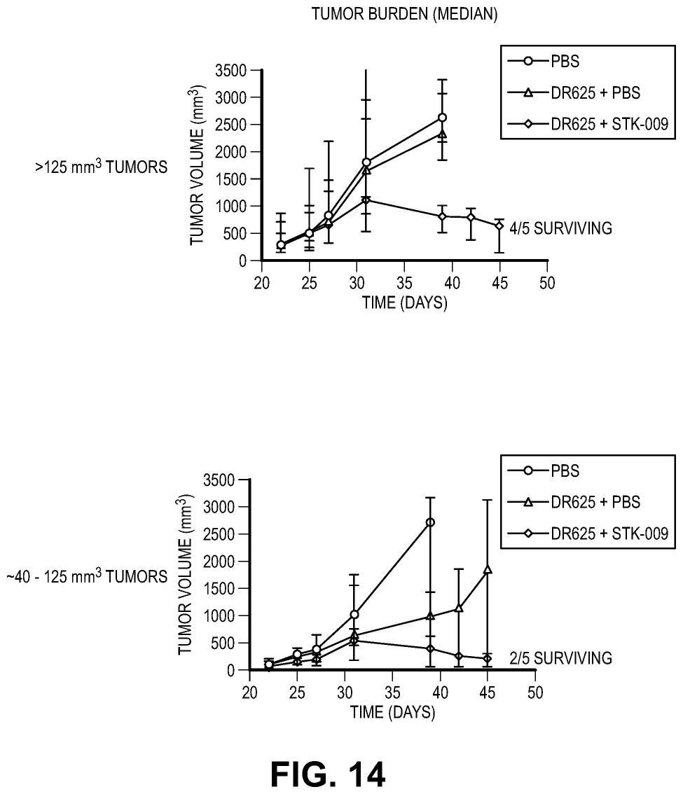

The foregoing study was repeated with the following modifications: (a) the initial implant was a subcutaneous implant of 2×10⁶ Huh7 cells expressing luciferase to facilitate imaging; (b) the Huh7 cells were implanted without Matrigel; (c) the dose of GPC3 orthoCAR T cells was increased to 5×10⁶ ortho GPC3 CAR T cells; (d) the STK-009 dose was administered 3 times per week. The data resulting from this study is presented in FIG. 14 of the attached drawings. As indicated, a significant fraction of the mice treated with the combination of the orthog GPC3 CAR T cells in combination with the ortho IL2 survived.

### 2. Huh7 Study B S2-21-019: DR625 in an Intraperitoneal Huh-7 In Vivo Model

This Huh7 Study B evaluated the effect of intraperitoneal administration of1×10⁶ Huh7 luciferase cells in NSG mice as discussed above. The treatment groups and treatments for this study are summarized in the following table:

| | | | | | Secondary | Secondary | |
|---|---|---|---|---|---|---|---|
| | | | Treatment | Secondary | Treatment | Treatment | |
| Group | Cells | Dose | Start Day | Treatment | Dose | Days | Mice/Group |
| 1 | n/a | n/a | 13 | PBS | | | 5 |
| 2 | DR625 | 5 × 10⁶ | 13 | PBS | | | 5 |
| 3 | DR625 | 5 × 10⁶ | 13 | STK-009 | 10 ug SQ | 3x week | 5 |
| 4 | DR625 | 2 × 10⁶ | 13 | PBS | | | 5 |
| 5 | DR625 | 2 × 10⁶ | 13 | STK-009 | 10 ug SQ | 3x week | 5 |

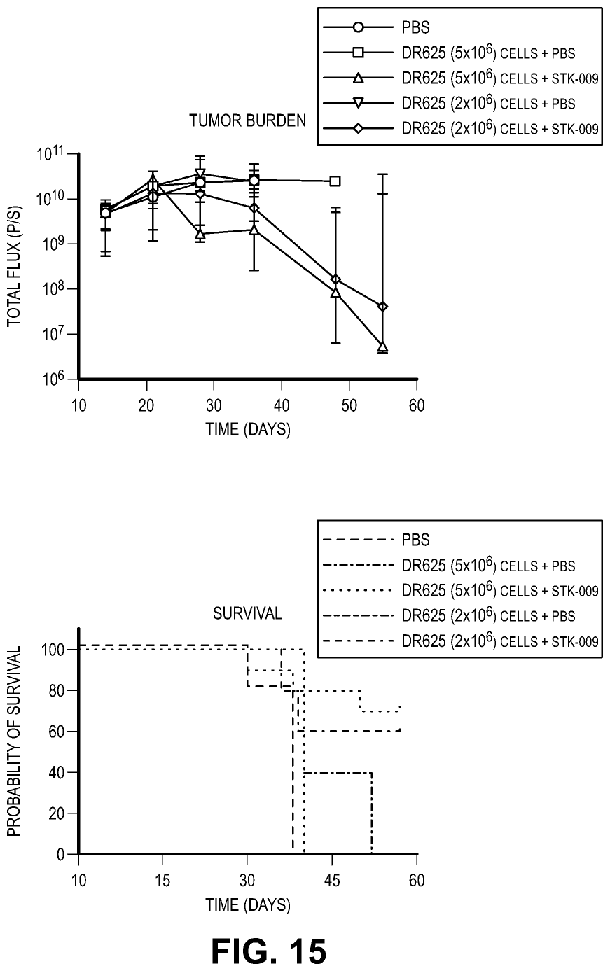

Tumor cell numbers were quantified twice weekly using an IVIS imager (Perkin Elmer). Mice were intraperitoneally injected with 100 ul of D-luciferin (15 mg/ml D-luciferin in PBS). Mice were put under anesthesia via controlled low-flow isoflurane exposure (Kent Scientific Somnosuite). A region of interest (ROI) was drawn around each individual mouse and total flux (photons/second) directly measuring luminescence was measured using Living Image software (Perkin Elmer). The results of this study are presented in FIG. 15 of the attached drawings.

As illustrated from the data presented in FIG. 15, the mice treated with the higher CAR-T cell dose (5×10⁶) cells demonstrated significant tumor regression. The mice treated with the lower CAR-T cell dose (2×10⁶) cells demonstrated significant control with tumors. The survival data presented in FIG. 14B demonstrates that the mice treated with the higher CAR-T cell dose (5×10⁶) cells demonstrated improved survival with most animals still alive at the conclusion of the study. The mice treated with the lower CAR-T cell dose (2×10⁶) cells demonstrated enhanced survival relative to control (PBS) but less than that observed at the higher CAR-T cell dose.

Collectively the foregoing data with respect to the treatment of Huh7 tumors demonstrates that an ortho GPC3 CAR T cell administered in combination with an ortho IL2 is effective in the treatment of tumors expressing low levels of GPC3.

### C. HepG2 Studies Rechallenge Studies

As previously discussed, one of the most challenging aspects of CAR-T therapy is that the initial response to the treatment is promising with very high response rates. However, over time, the patients relapse, and the cancer recurs even with IL2 supportive therapy. Also, the persistence of active CAR T cells from existing therapies is poor with the majority of the activated CAR T cells administered becoming non-efficacious through exhaustion or other mechanisms.

To evaluate the potential to treat relapse in a GPC3+ tumors, animals that were tumor free upon completion of the in the foregoing HepG2 studies described above were rechallenged by the administration of additional HepG2 tumor cells and treated solely with an ortho IL2 without the administration of ortho GPC3 CAR T cells.

1. HepG2 Rechallenge Study A: Rechallenge of Tumor Free Mice from Study HepG2 Study A with Additional Tumor and Just STK-009

In this study mice that survived from the treatment groups in HepG2 Study A discussed above were rechallenged by the administration of an additional quantity (2×10⁶) of HepG2 tumor cells on Day 55 following first tumor implant and then again on Day 80. The study design and treatment schedule is summarized in the table below.

| | | | Secondary | | Treatment | |
|---|---|---|---|---|---|---|
| Group | Mice | Previous treatment | Treatment | Dose | Days* | Mice/Efficacy |
| 1 | Naive | None | PBS | — | | 5 |
| 2 | S2-21-001 | DR626 + STK-009 | STK-009 | 10 ug SQ | Day −15, −8, | 7 or 8 |
| | Group 3 | (2E6 Total T cells) | | | 0, 23, 25, 27, | |
| | | | | | 38, and 49 | |
| 3 | S2-21-001 | DR625 + STK-009 | STK-009 | 10 ug SQ | Day −15, −8, | 4 |
| | Group 5 | (1E6 Total T cells) | | | 0, 23, 25, 27, | |
| | | | | | 38, and 49 | |

*beginning on time of rechallenged implant (Day 0)

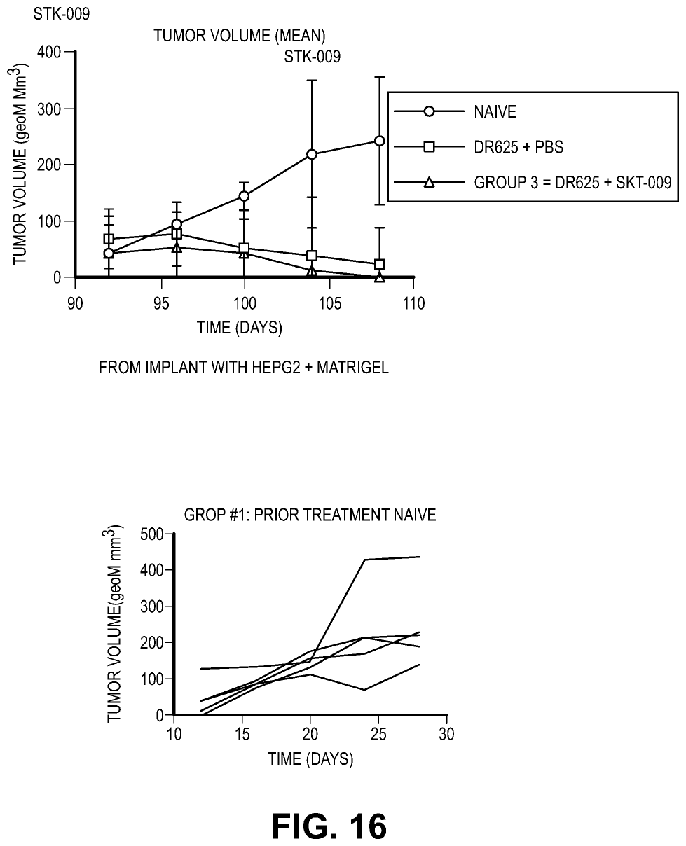

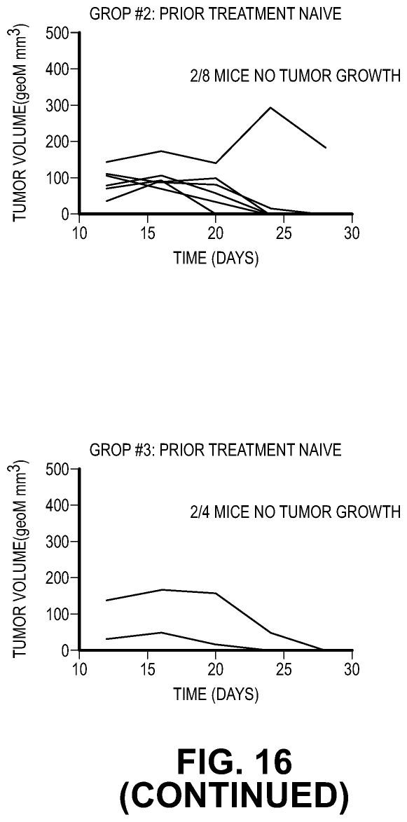

The data from this experiment is presented in FIG. 16 of the attached drawings. As can be seen from data presented, the addition of STK-009 alone in the absence of any additional CAR-T cells, results tumor regression. This data demonstrates that in subjects previously treated with an ortho GPC3 CAR T cell who relapse and whose tumors recur may be successfully treated by the administration of the ortho IL2 without the additional CAR T cell therapy. This data suggests that the GPC3 CAR T cells which were previously administered in the HepG2 Study A were able to be reinvigorated by the administration of ortho IL2 (STK-009) and retained antitumor efficacy.

2. HepG2 Rechallenge Study B S2-21-012: Rechallenge of Tumor Free Mice from Study HepG2 Study B S2-21-009 with Additional Tumor and Just STK-009

A second rechallenge study was done S2-21-012 HepG2 rechallenge study was conducted using the mice that survived from HepG2 Study B. In this study the surviving mice from the previous study were subcutaneously administered 2.5E6 cells HepG2-luciferase cells in Matrigel. The study design and treatment protocols are summarized in the following table.

| | | Previous | Secondary | | |
|---|---|---|---|---|---|
| Group | Mice | treatment | Treatment | Dose | Mice/Efficacy |
| 1 | Naive | None | PBS | PBS | 10 |
| 2 | Group #3 From | 8.5 × 10⁵ | PBS | PBS | 4 |
| | HepG2 Study B | | | | |
| 3 | Group #3 From | 8.5 × 10⁵ | STK-009 | 10 micrograms | 5 |
| | HepG2 Study B | | | twice per week* | |

*STK-009 was administered beginning on the day of reimplantation of tumor cells(Day 0).

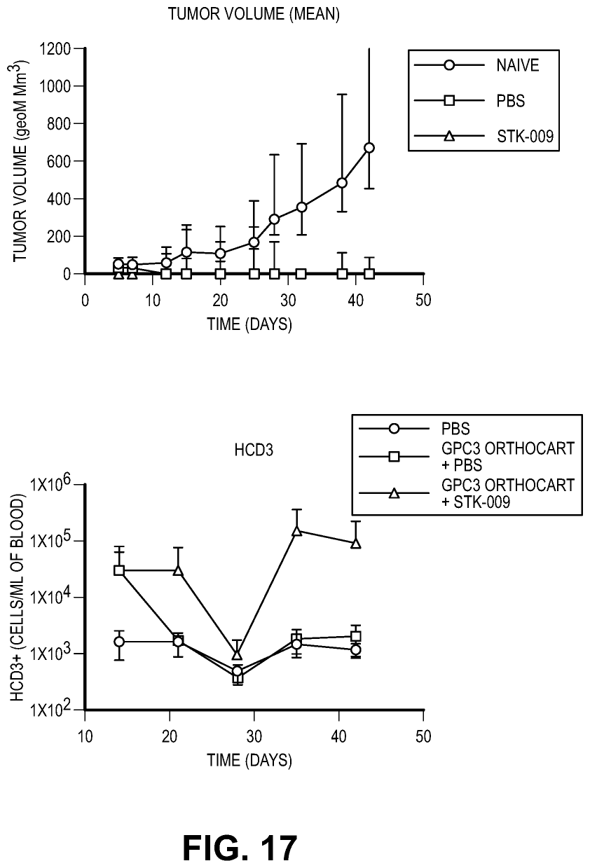

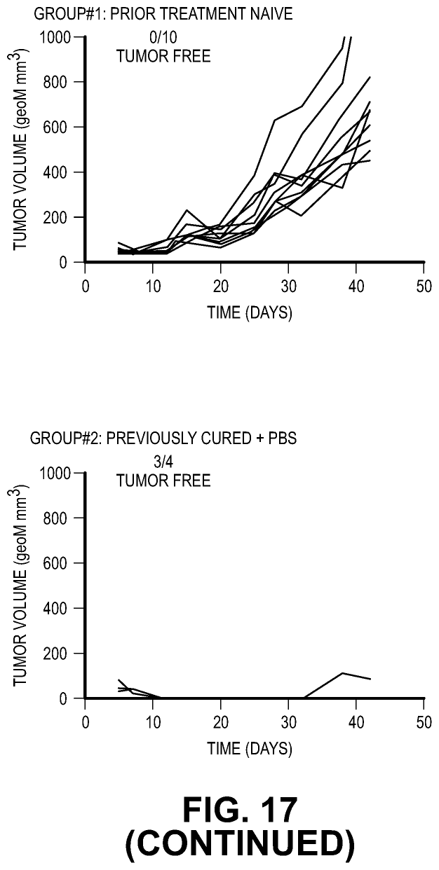

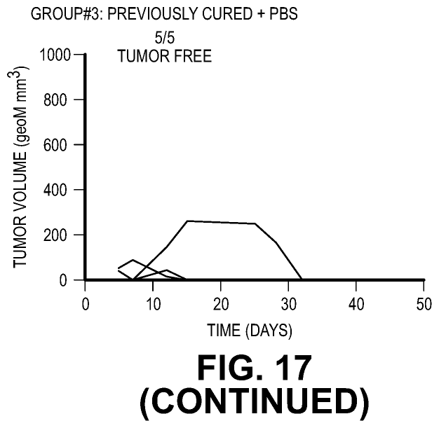

The data generated from this study is provided in FIG. 17. As can be seen from the data presented, the majority of mice previously cured with the ortho GPC3 CAR T cells were able to suppress rechallenged tumor growth. Consistent with the previous study, this data demonstrates that in subjects previously treated with an ortho GPC3 CAR T cell who relapse and whose tumors recur may be successfully treated by the administration of the ortho IL2 without the additional CAR T cell therapy.

### A. GPC3 Chimeric Antigen Receptors or GPC3 CARs:

The terms “chimeric antigen receptor” and “CAR” are used interchangeably to refer to a chimeric polypeptide comprising multiple functional domains arranged from amino to carboxy terminus in the sequence: (a) an extracellular domain (ECD) comprising an antigen binding domain (ABD), and optionally comprising a “hinge” domain, (b) a transmembrane domain (TM); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs may be prepared in accordance with principles well known in the art. See e.g., Eshhar, et al. (U.S. Pat. No. 7,741,465 B1 issued Jun. 22, 2010); Sadelain, et al. (2013) Cancer Discovery 3 (4): 388-398; Campana and Imai (U.S. Pat. No. 8,399,645 issued Mar. 19, 2013) Jensen and Riddell (2015) Current Opinions in Immunology 33:9-15; Gross, et al. (1989) PNAS (USA) 86 (24): 10024-10028; Curran, et al. (2012) J Gene Med 14 (6): 405-15; Brogdon, et al. (U.S. Pat. No. 10,174,095 issued Jan. 8, 2019) Guedan, et al. (2019) Engineering and Design of Chimeric Antigen Receptors Molecular Therapy: Methods & Clinical Development Vol. 12:145-156.

The present disclosure provides a GPC3 CAR which is a CAR wherein the antigen binding domain (ABD) of the CAR specifically binds to GPC3. In some embodiments, the ABD of the GPC3 CARs of the present disclosure is a single domain antibody (sdAb) that selectively binds to hGPC3 (“anti-hGPC3sdAb”). In some embodiments, the anti-hGPC3sdAb of the present disclosure is a single domain antibody that selectively binds to mature human GPC3 (amino acids 25-580 of SEQ ID NO:1). In some embodiments, the anti-hGPC3sdAb of the present disclosure is a single domain antibody that selectively binds human GPC3 beta subunit (SEQ ID NO:2).

In some embodiments, the anti-GPC3sdAb is an scFv (anti-hGPC3scFv). An scFv is a polypeptide comprised of the variable regions of the immunoglobulin heavy and light chain of an antibody covalently connected by a peptide linker (Bird, et al. (1988) Science 242:423-426; Huston, et al. (1988) PNAS (USA) 85:5879-5883; S-z Hu, et al. (1996) Cancer Research, 56, 3055-3061; Ladner, U.S. Pat. No. 4,946,778 issued Aug. 7, 1990). In some embodiments, the anti-hGPC3scFv selectively binds to mature human GPC3 (amino acids 25-580 of SEQ ID NO:1). In some embodiments, the anti-hGPC3scFv selectively binds to the human GPC3 beta subunit (SEQ ID NO:2). The preparation of an anti-GPC3 antigen ScFv proceeds initially by generating a monoclonal antibody against GPC3 or an antigenic fragment thereof. The generation of monoclonal antibodies and isolation of hybridomas is a technique well known to those of skill in the art. See e.g. Monoclonal Antibodies: A Laboratory Manual, Second Edition, Chapter 7 (E. Greenfield, Ed. 2014 Cold Spring Harbor Press). Generation of anti-hGPC3 monoclonal antibodies proceeds by the immunization of an immune competent species including but not limited to mice, rabbits, horses, pigs, camels, llamas, dromedaries, or sharks with a hGPC3 polypetide or an antigenic fragment thereof. In some embodiments, the immunogen used to generate the immune response is human glypican-3 (SEQ ID NO:1) or antigenic fragment thereof. In some embodiments, the immunogen is the human glypican-3 beta subunit (SEQ ID NO:2) or antigenic fragment thereof. Immune response to the immunogen may be enhanced through co-administration of the immunogen with adjuvants well known in the art including but not limited to alum, aluminum salts, Freund's complete adjuvant (FCA), and SP-21.

The antibodies generated through the immunization process may be optimized to select for antibodies possessing particular desirable characteristics through techniques well known in the art such as phage display and directed evolution. See, e.g. Barbas, et al. (1991) PNAS (USA) 88:7978-82; Ladner, et al. U.S. Pat. No. 5,223,409 issued Jun. 29, 1993; Stemmer, W. (1994) Nature 370:389-91; Garrard U.S. Pat. No. 5,821,047 issued Oct. 13, 1998; Camps, et al. (2003) PNAS (USA) 100 (17): 9727-32; Dulbecco U.S. Pat. No. 4,593,002 issued Jun. 3, 1986; McCafferty U.S. Pat. No. 6,806,079 issued Oct. 19, 2004; McCafferty, U.S. Pat. No. 7,635,666 issued Dec. 22, 2009; McCafferty, U.S. Pat. No. 7,662,557 issued Feb. 16, 2010; McCafferty, U.S. Pat. No. 7,723,271 issued May 25, 2010; and/or McCafferty U.S. Pat. No. 7,732,377.

As an alternative to immunization with a GPC3 immunogen, antiGPC3scFvs may also be generated based on known anti-GPC3 antibody sequences. See, e.g. The Protein Protocols Handbook, John M. Walker, Ed. (2002) Humana Press Section 150 “Bacterial Expression, Purification and Characterization of Single-Chain Antibodies” Kipriyanov, S.; Feng, et al. Therapeutically targeting glypican-3 via a conformation-specific single-domain antibody in hepatocellular carcinoma (2013) PNAS (USA) 1110 (12): E1083-E1091. A variety of anti-GPC3 antibodies have been described in the literature. The CDRs of such antibodies may also be grafted onto scFv frameworks, particularly human or humanized scFv frameworks, to generate humanized anti-hGPC3scFvs based on techniques known in the art. Examples of known anti-GPC3 antibodies and their CDRs (based on Kabat numbering conventions) useful in the preparation of antiGPC3scFvs are summarized in the following table:

Antibodies that selectively bind to GPC3 and subunit B of GPC3 are known in the art including but not limited to GC33 (Nakano, et al., U.S. Pat. No. 7,919,086 and Nakano, et al (2009) Biochem Biophys Res Comm 378 (2): 279-84), YP7, YP8, YP9, YP9.1, 32A9 are described in Liu, et al (2020) J Translational Medicine 18, article number 295 published Aug. 3, 2020 and Chinese Patent CN109021108B granted Jun. 25, 2019.

In one embodiment, the ABD of the GPC3 CAR is an scFv comprising the CDRs derived from the GC33 antibody. In some embodiments the s ABD of the GPC3 CAR is an scFv comprising heavy chain CDRs 1, 2, and 3 independently have at least 90%, 91%, 92%, 93%, 94%, 95%, 96%, 97%, 98%, 99% sequence identity to SEQ ID NOS: 12, 13 and 115, 16, 17 respectively and light chain CDRs 1, 2, and 3 independently have at least 90%, 91%, 92%, 93%, 94%, 95%, 96%, 97%, 98%, 99% sequence identity to SEQ ID NOS: 12, 13 and 14 respectively. In some embodiments the s ABD of the GPC3 is an scFv wherein the heavy chain CDRs 1, 2, and 3 are identical to SEQ ID NOS: 12, 13 and 14 respectively and wherein the light chain CDRs 1, 2, and 3 are identical to SEQ ID NOS: 15, 16, 17 respectively.

In some embodiments the ABD of the GPC3 CAR is an scFv comprising a heavy chain variable region having CDRs 1, 2 and 3 comprised of the amino acid sequences DYEMH (SEQ ID NO: 12), ALDPKTGDTAYSQKFKG (SEQ ID NO: 13) and FYSYTY (SEQ ID NO: 14), respectively, and a light chain variable region having CDRs 1, 2 and 3 comprised of the amino acid sequences RSSQSLVHSNANTYLH (SEQ ID NO: 15) KVSNRFS (SEQ ID NO: 16), and SQNTHVPPT (SEQ ID NO: 17).

As noted above, the heavy and light chain regions of the scFv are joined via a polypeptide linker. In some embodiments, the linker between the heavy chain variable region and the light chain region is a polypeptide linker having 3, 4, 5, 6, 7, 8, 9, 10, 11, 12, 13, 14, 15, 16, 17, 18, 19, 20, 21, 22, 23, 24, 25, 26, 27, 28, 29 or 30 amino acids. In some embodiments, the linker between the heavy and light chain variable regions of the scFv is a polypeptide of 15 amino acids. In some embodiments, the linker between the heavy and light chain variable regions of the scFv is a polypeptide having the amino acid sequence GSTSGSGKPGSGEGSTKG (SEQ ID NO:18) referred to in the art as the Whitlow linker as described in U.S. Pat. No. 5,856,456.

In some embodiments the linker is a polypeptide comprised primarily of glycine (G) and serine(s) resides referred to in the art as “GS” linkers. Glycine and glycine-serine polymers are relatively unstructured, and therefore may serve as a neutral tether between components. Examples of glycine polymers include (G) n, glycine-alanine polymers, alanine-serine polymers, glycine-serine polymers (for example, (GmSo) n, (GSGGS) n, (GmSoGm) n, (GmSoGmSoGm) n, (GSGGSm) n, (GSGSmG) n and (GGGSm) n, and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers. One example of a GS linker useful in the practice of the present disclosure is a polypeptide having the amino acid sequence GGGGSGGGGSGGGGS (SEQ ID NO:49) abbreviated herein “G4Sx3”

In some embodiments ABD of the GPC3 CAR is an scFv comprising a heavy chain variable region having CDRs 1, 2 and 3 comprised of the amino acid sequences DYEMH (SEQ ID NO: 12), ALDPKTGDTAYSQKFKG (SEQ ID NO: 13) and FYSYTY (SEQ ID NO: 14), respectively, and a light chain variable region having CDRs 1, 2 and 3 comprised of the amino acid sequences RSSQSLVHSNANTYLH (SEQ ID NO: 15) KVSNRFS (SEQ ID NO: 16), and SQNTHVPPT (SEQ ID NO: 17) wherein the heavy and light chain variable regions are joined via a polypeptide linker having the amino acid sequence GSTSGSGKPGSGEGSTKG (SEQ ID NO:33).

In some embodiments the ABD of the GPC3 CAR is an scFv comprising a heavy chain variable region having CDRs 1, 2 and 3 comprised of the amino acid sequences DYEMH (SEQ ID NO: 12), ALDPKTGDTAYSQKFKG (SEQ ID NO: 13) and FYSYTY (SEQ ID NO: 14), respectively, and a light chain variable region having CDRs 1, 2 and 3 comprised of the amino acid sequences RSSQSLVHSNANTYLH (SEQ ID NO: 15) KVSNRFS (SEQ ID NO: 16), and SQNTHVPPT (SEQ ID NO: 17) wherein the heavy and light chain variable regions are joined via a polypeptide linker having the amino acid sequence GSTSGSGKPGSGEGSTKG (SEQ ID NO:18), the scFv having at least 90%, 91%, 92%, 93%, 94%, 95%, 96%, 97%, 98%, 99% sequence identity the amino acid sequence:

(SEQ ID NO: 11)
DVVMTQSPLSLPVTPGEPASISCRSSQSLVHSNANTYLHWYLQKP
GQSPQLLIYKVSNRFSGVPDRFSGSGSGTDFTLKISRVEAEDVGV
YYCSQNTHVPPTFGQGTKLEIKRGSTSGSGKPGSGEGSTKGQVQL
VQSGAEVKKPGASVKVSCKASGYTFTDYEMHWVRQAPGQGLEWMG
ALDPKTGDTAYSQKFKGRVTLTADESTSTAYMELSSLRSEDTAVY
YCTRFYSYTYWGQGTLVTVSS.

In some embodiments the anti-GPC3 sdAb is an scFv comprising a heavy chain variable region having CDRs 1, 2 and 3 comprised of the amino acid sequences DYEMH (SEQ ID NO: 12), ALDPKTGDTAYSQKFKG (SEQ ID NO: 13) and FYSYTY (SEQ ID NO: 14), respectively, and a light chain variable region having CDRs 1, 2 and 3 comprised of the amino acid sequences RSSQSLVHSNANTYLH (SEQ ID NO: 15) KVSNRFS (SEQ ID NO: 16), and SQNTHVPPT (SEQ ID NO: 17) wherein the heavy and light chain variable regions are joined via a polypeptide linker having the amino acid sequence GGGGGGGGSGGGGS (SEQ ID NO:49) In some embodiments the anti-GPC3 sdAb is an scFv comprising a heavy chain variable region having CDRs 1, 2 and 3 comprised of the amino acid sequences DYEMH (SEQ ID NO: 12), ALDPKTGDTAYSQKFKG (SEQ ID NO: 19) and FYSYTY (SEQ ID NO: 14), respectively, and a light chain variable region having CDRs 1, 2 and 3 comprised of the amino acid sequences RSSQSLVHSNANTYLH (SEQ ID NO: 15) KVSNRFS (SEQ ID NO: 16), and SQNTHVPPT (SEQ ID NO: 17) wherein the heavy and light chain variable regions are joined via a polypeptide linker having the amino acid sequence GGGGSGGGGSGGGGS (SEQ ID NO:49), the scFv having at least 90%, 91%, 92%, 93%, 94%, 95%, 96%, 97%, 98%, 99% sequence identity the amino acid sequence:

(SEQ ID NO: 10)
DVVMTQSPLSLPVTPGEPASISCRSSQSLVHSNANTYLHWYLQKP
GQSPQLLIYKVSNRFSGVPDRFSGSGSGTDFTLKISRVEAEDVGV
YYCSQNTHVPPTFGQGTKLEIKRGGGGGGGGSGGGGSQVQLVQSG
AEVKKPGASVKVSCKASGYTFTDYEMHWVRQAPGQGLEWMGALDP
KTGDTAYSQKFKGRVTLTADESTSTAYMELSSLRSEDTAVYYCTR
FYSYTYWGQGTLVTVSS

### Signal Peptide (SP):

To facilitate surface expression the GPC3 CAR, the nucleic acid sequence encoding the GPC3 CAR may further encode a signal peptide which facilitates the surface expression of the GPC3 CAR when expressed in a mammalian cell and which is post-translationally cleaved from the polypeptide. The signal peptide may be derived from naturally occurring signal peptides of surface expressed proteins or synthetic. In one embodiment of the invention where the signal peptide is derived from a naturally occurring signal peptide of a surface expressed protein, the signal peptide of the GPC3 CAR is the signal peptide selected from the group consisting of the human CD8a signal peptide, the human serum albumin (HSA) signal peptide, the prolactin albumin signal peptide, the human IL2 signal peptide, human trypsinogen-2, human CD-5 signal peptide, the human immunoglobulin kappa light chain signal peptide, and functional derivatives thereof. Particular amino acid substitutions in naturally occurring signal peptide sequences to increase secretion efficiency using signal peptides are described in Stern, et al. (2007) Trends in Cell and Molecular Biology 2:1-17 and Kober, et al. (2013) Biotechnol Bioeng. 1110 (4): 1164-73. In the alternative to the use of a signal peptide derived from a naturally occurring source, the signal peptide may be a synthetic sequence prepared in accordance established principles. See e.g., Nielsen, et al. (1997) Protein Engineering 10 (1): 1-6; Bendtsen, et al (2004) J. Mol. Biol 340 (4): 783-795; Petersen, et al (2011) Nature Methods 8:785-796. In one embodiment as exemplified herein, the GPC3 CAR comprises the human CD8a signal peptide having the amino acid sequence MALPVTALLLPLALLLHAARP (SEQ ID NO:20).

In some embodiments, ABD of the GPC3 CAR is a VHH that specifically binds to GPC3 (SEQ ID NO:1) or a fragment thereof (e.g. SEQ ID NO:2). VHHs are the heavy domain fragment of the antibodies generated by Camelidae mammals (e.g., camels, llamas, dromedary, alpaca, and guanaco) which are naturally devoid of light chains. An typical VHH has a molecular weight of approximately 12-15 kDa which is much smaller than traditional mammalian IgG class antibodies (150-160 kDa) composed of two heavy chains and two light chains. Similar to more conventional IgG class antibodies generated in other mammalian species, camelid antibodies selectively to a specific antigen. Anti-GPC3 camelid antibodies from which antiGPC3VHH 1 are similarly generated by the immunization of Camelidae with proceeds by the immunization of an immune competent animal with a hGPC3 polypeptide or an antigenic fragment thereof. In some embodiments, the antiGPC3 VHH binds to mature human GPC3 (amino acids 25-580 of SEQ ID NO:1) derived from an camelid antibody obtained by immunization of a camelid with a polypeptide sequence comprising amino acids 25-580 of SEQ ID NO:1. In some embodiments, the antiGPC3 VHH is an VHH (anti-GPC3-VHH) specifically binds to the human GPC3 beta subunit (SEQ ID NO:2) derived from an camelid antibody obtained by immunization of a camelid with a polypeptide sequence comprising amino acids GPC3 beta subunit (SEQ ID NO:2).

GPC3 CARs of the present disclosure further provide a transmembrane spanning domain linking the ABD (optionally including polypeptide linker) to the intracellular domain (ICD) of the GPC3 CAR. The transmembrane spanning domain is comprised of any sequence which is thermodynamically stable in a eukaryotic cell membrane. Transmembrane spanning domains useful in construction of GPC3 CARs useful in the practice of the present invention are comprised of approximately 20 amino acids favoring the formation having an alpha-helical secondary structure. The transmembrane spanning domain may be derived from the transmembrane domain of a naturally occurring membrane spanning protein. Alternatively, the transmembrane domain may be synthetic. In designing synthetic transmembrane domains, amino acids favoring alpha-helical structures are preferred. Amino acids favoring the formation of alpha-helices are well known in the art. See e.g., Pace, et al. (1998) Biophysical Journal 75:422-427. In some embodiments, the CAR transmembrane domain may be derived from the transmembrane domain from type I membrane spanning proteins including but not limited to CD35, CD4, CD8, CD28. In some embodiments, the transmembrane spanning domain is the human CD28 transmembrane domain corresponding to amino acids 132-157 of the human CD28 precursor protein, numbered in accordance with human CD28 precursor protein including the signal peptide UniProt P10747.

The cytoplasmic domain of the GPC3 CAR comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor zeta-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ-chain, cytoplasmic domain of CD134 (also referred to as OX40 and TNFRSF4), FcεR1γ and β chains, MB1 (Iga) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

In some embodiments, the GPC3 CAR may also provide a co-stimulatory domain. The term “co-stimulatory domain”, refers to a stimulatory domain, typically an endodomain, of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. (2013) Nat Rev Immunol 13 (4): 227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

The ordinarily skilled artisan is aware of other co-stimulatory domains that may be used in conjunction with the teachings of the present disclosure.

In one embodiment of the disclosure, the ICD of the GPC3-CAR comprises the cytoplasmic domain of CD28 and a portion of the intracellular signaling domain of CD3 ζ.

In one embodiment of the invention, the ICD of the GPC3-CAR comprises the cytoplasmic domain of 4-1BB and a portion of the intracellular signaling domain of CD3 ζ.

In another embodiment, the ICD of the GPC3-CAR comprises the signaling domain of CD3-zeta and the signaling domain of CD28 and CD137.

GPC3 CARs of the present disclosure may optionally include one or more polypeptide spacers linking the functional domains of the CAR, in particular the linkage between the GPC3-ABD to the transmembrane spanning domain of the CAR. Although not an essential element of the CAR structure, the inclusion of a spacer domain is generally considered desirable to facilitate antigen recognition by the ABD. Moritz and Groner (1995) Gene Therapy 2 (8) 539-546. There is no particular sequence of amino acids that is necessary to achieve the spacer function but the typical properties of the spacer are flexibility to enable freedom of movement of the ABD to facilitate targeting antigen recognition. Similarly, it has been found that there is there is substantial leniency in spacer length while retaining CAR function. Jensen and Riddell (2014) Immunol. Review 257 (1) 127-144. Sequences useful as spacers in the construction of CARs useful in the practice of the present invention include but are not limited to the hinge region of IgG1, the immunoglobulin1CH2-CH3 region, IgG4 hinge-CH2-CH3, IgG4 hinge-CH3, and the IgG4 hinge. The hinge and transmembrane domains may be derived from the same molecule such as the hinge and transmembrane domains of CD8-alpha. Imai, et al. (2004) Leukemia 18 (4): 676-684.

The present disclosure provides GPC3 CARs which may be useful in the preparation of orthogonal GPC3 CAR T cells. In one embodiment, the GPC3 CAR comprises the amino acid sequence the hCD8a signal peptide (italics), a GC33 scFv with a Whitlow linker as an antigen binding domain (Whitlow Linker underlined), a polypeptide sequence possessing a small sequence of the extracellular domain of hCD28, the transmembrane and co-stimulatory domain and the human CD3zeta domain, the GPC3 CAR at least 90%, 91%, 92%, 93%, 94%, 95%, 96%, 97%, 98%, 99% sequence identity to the amino acid sequence:

MALPVTALLLPLALLLHAARPDVVMTQSPLSLPVTPGEPASISCR
SSQSLVHSNANTYLHWYLQKPGQSPQLLIYKVSNRFSGVPDRFSG
SGSGTDFTLKISRVEAEDVGVYYCSQNTHVPPTFGQGTKLEIKRG
STSGSGKPGSGEGSTKGQVQLVQSGAEVKKPGASVKVSCKASGYT
FTDYEMHWVRQAPGQGLEWMGALDPKTGDTAYSQKFKGRVTLTAD
ESTSTAYMELSSLRSEDTAVYYCTRFYSYTYWGQGTLVTVSSIEV
MYPPPYLDNEKSNGTIIHVKGKHLCPSPLFPGPSKPFWVLVVVGG
VLACYSLLVTVAFIIFWVRSKRSRLLHSDYMNMTPRRPGPTRKHY
QPYAPPRDFAAYRSRVKFSRSADAPAYQQGQNQLYNELNLGRREE
YDVLDKRRGRDPEMGGKPRRKNPQEGLYNELQKDKMAEAYSEIGM
KGERRRGKGHDGLYQGLSTATKDTYDALHMQALPPR

In one embodiment, the GPC3 CAR comprises the amino acid sequence of the hCD8a signal peptide (italics), a GC33 scFv with a G4Sx3 linker as an antigen binding domain (G4Sx3 linker underlined), a polypeptide sequence possessing a small sequence of the extracellular domain of hCD28, the transmembrane and co-stimulatory domain and the human CD3zeta domain, the GPC3 CAR having at least 90%, 91%, 92%, 93%, 94%, 95%, 96%, 97%, 98%, 99% sequence identity to the amino acid sequence:

MALPVTALLLPLALLLHAARPDVVMTQSPLSLPVTPGEPASISCR
SSQSLVHSNANTYLHWYLQKPGQSPQLLIYKVSNRFSGVPDRFSG
SGSGTDFTLKISRVEAEDVGVYYCSQNTHVPPTFGQGTKLEIKRG
GGGSGGGGSGGGGSQVQLVQSGAEVKKPGASVKVSCKASGYTFTD
YEMHWVRQAPGQGLEWMGALDPKTGDTAYSQKFKGRVTLTADEST
STAYMELSSLRSEDTAVYYCTRFYSYTYWGQGTLVTVSSIEVMYP
PPYLDNEKSNGTIIHVKGKHLCPSPLFPGPSKPFWVLVVVGGVLA
CYSLLVTVAFIIFWVRSKRSRLLHSDYMNMTPRRPGPTRKHYQPY
APPRDFAAYRSRVKFSRSADAPAYQQGQNQLYNELNLGRREEYDV
LDKRRGRDPEMGGKPRRKNPQEGLYNELQKDKMAEAYSEIGMKGE
RRRGKGHDGLYQGLSTATKDTYDALHMQALPPR

In one embodiment, the GPC3 CAR comprises the amino acid sequence of the hCD8a signal peptide (italics), a GC33 scFv with a Whitlow linker as an antigen binding domain (Whitlow linker underlined), the hCD8a hinge and transmembrane domain, the human 4-1BB co-stimulatory domain at least 90%, 91%, 92%, 93%, 94%, 95%, 96%, 97%, 98%, 99% sequence identity the amino acid sequence:

MALPVTALLLPLALLLHAARPDVVMTQSPLSLPVTPGEPASISCR
SSQSLVHSNANTYLHWYLQKPGQSPQLLIYKVSNRFSGVPDRFSG
SGSGTDFTLKISRVEAEDVGVYYCSQNTHVPPTFGQGTKLEIKRG
STSGSGKPGSGEGSTKGQVQLVQSGAEVKKPGASVKVSCKASGYT
FTDYEMHWVRQAPGQGLEWMGALDPKTGDTAYSQKFKGRVTLTAD
ESTSTAYMELSSLRSEDTAVYYCTRFYSYTYWGQGTLVTVSSTTT
PAPRPPTPAPTIASQPLSLRPEACRPAAGGAVHTRGLDFACDIYI
WAPLAGTCGVLLLSLVITLYCKRGRKKLLYIFKQPFMRPVQTTQE
EDGCSCRFPEEEEGGCELRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPR

In one embodiment, the GPC3 CAR comprises the amino acid sequence at least 90%, 91%, 92%, 93%, 94%, 95%, 96%, 97%, 98%, 99% sequence identity the amino acid sequence:

(SEQ ID NO: 37)
MALPVTALLLPLALLLHAARPDVVMTQSPLSLPVTPGEPASISCR
SSQSLVHSNANTYLHWYLQKPGQSPQLLIYKVSNRFSGVPDRFSG
SGSGTDFTLKISRVEAEDVGVYYCSQNTHVPPTFGQGTKLEIKRG
STSGSGKPGSGEGSTKGQVQLVQSGAEVKKPGASVKVSCKASGYT
FTDYEMHWVRQAPGQGLEWMGALDPKTGDTAYSQKFKGRVTLTAD
ESTSTAYMELSSLRSEDTAVYYCTRFYSYTYWGQGTLVTVSSAAA
IEVMYPPPYLDNEKSNGTIIHVKGKHLCPSPLFPGPSKPFWVLVV
VGGVLACYSLLVTVAFIIFWVRSKRSRLLHSDYMNMTPRRPGPTR
KHYQPYAPPRDFAAYRSRVKFSRSADAPAYQQGQNQLYNELNLGR
REEYDVLDKRRGRDPEMGGKPRRKNPQEGLYNELQKDKMAEAYSE
IGMKGERRRGKGHDGLYQGLSTATKDTYDALHMQALPPRGSGEGR
GSLLTCGDVEENPGPMAAPALSWRLPLLILLLPLATSWASAAVNG
TSQFTCFYNSRANISCVWSQDGALQDTSCQVHAWPDRRRWNQTCE
LLPVSQASWACNLILGAPDSQKLTTVDIVTLRVLCREGVRWRVMA
IQDFKPFENLRLMAPISLQVVHVETHRCNISWEISQASDFFERHL
EFEARTLSPGHTWEEAPLLTLKQKQEWICLETLTPDTQYEFQVRV
KPLQGEFTTWSPWSQPLAFRTKPAALGKDTIPWLGHLLVGLSGAF
GFIILVYLLINCRNTGPWLKKVLKCNTPDPSKFFSQLSSEHGGDV
QKWLSSPFPSSSFSPGGLAPEISPLEVLERDKVTQLLLQQDKVPE
PASLSSNHSLTSCFTNQGYFFFHLPDALEIEACQVYFTYDPYSEE
DPDEGVAGAPTGSSPQPLQPLSGEDDAYCTFPSRDDLLLFSPSLL
GGPSPPSTAPGGSGAGEERMPPSLQERVPRDWDPQPLGPPTPGVP
DLVDFQPPPELVLREAGEEVPDAGPREGVSFPWSRPPGQGEFRAL
NARLPLNTDAYLSLQELQGQDPTHLV.

In one embodiment, the GPC3 CAR comprises the amino acid sequence at least 90%, 91%, 92%, 93%, 94%, 95%, 96%, 97%, 98%, 99% sequence identity the amino acid sequence:

(SEQ ID NO: 38)
MAAPALSWRLPLLILLLPLATSWASAAVNGTSQFTCFYNSRANIS
CVWSQDGALQDTSCQVHAWPDRRRWNQTCELLPVSQASWACNLIL
GAPDSQKLTTVDIVTLRVLCREGVRWRVMAIQDFKPFENLRLMAP
ISLQVVHVETHRCNISWEISQASDFFERHLEFEARTLSPGHTWEE
APLLTLKQKQEWICLETLTPDTQYEFQVRVKPLQGEFTTWSPWSQ
PLAFRTKPAALGKDTIPWLGHLLVGLSGAFGFIILVYLLINCRNT
GPWLKKVLKCNTPDPSKFFSQLSSEHGGDVQKWLSSPFPSSSFSP
GGLAPEISPLEVLERDKVTQLLLQQDKVPEPASLSSNHSLTSCFT
NQGYFFFHLPDALEIEACQVYFTYDPYSEEDPDEGVAGAPTGSSP
QPLQPLSGEDDAYCTFPSRDDLLLFSPSLLGGPSPPSTAPGGSGA
GEERMPPSLQERVPRDWDPQPLGPPTPGVPDLVDFQPPPELVLRE
AGEEVPDAGPREGVSFPWSRPPGQGEFRALNARLPLNTDAYLSLQ
ELQGQDPTHLVGSGEGRGSLLTCGDVEENPGPMALPVTALLLPLA
LLLHAARPDVVMTQSPLSLPVTPGEPASISCRSSQSLVHSNANTY
LHWYLQKPGQSPQLLIYKVSNRFSGVPDRFSGSGSGTDFTLKISR
VEAEDVGVYYCSQNTHVPPTFGQGTKLEIKRGSTSGSGKPGSGEG
STKGQVQLVQSGAEVKKPGASVKVSCKASGYTFTDYEMHWVRQAP
GQGLEWMGALDPKTGDTAYSQKFKGRVTLTADESTSTAYMELSSL
RSEDTAVYYCTRFYSYTYWGQGTLVTVSSAAAIEVMYPPPYLDNE
KSNGTIIHVKGKHLCPSPLFPGPSKPFWVLVVVGGVLACYSLLVT
VAFIIFWVRSKRSRLLHSDYMNMTPRRPGPTRKHYQPYAPPRDFA
AYRSRVKFSRSADAPAYQQGQNQLYNELNLGRREEYDVLDKRRGR
DPEMGGKPRRKNPQEGLYNELQKDKMAEAYSEIGMKGERRRGKGH
DGLYQGLSTATKDTYDALHMQALPPR

In one embodiment, the GPC3 CAR comprises the amino acid sequence at least 90%, 91%, 92%, 93%, 94%, 95%, 96%, 97%, 98%, 99% sequence identity the amino acid sequence:

(SEQ ID NO: 39)
MALPVTALLLPLALLLHAARPDVVMTQSPLSLPVTPGEPASISCR
SSQSLVHSNANTYLHWYLQKPGQSPQLLIYKVSNRFSGVPDRFSG
SGSGTDFTLKISRVEAEDVGVYYCSQNTHVPPTFGQGTKLEIKRG
STSGSGKPGSGEGSTKGQVQLVQSGAEVKKPGASVKVSCKASGYT
FTDYEMHWVRQAPGQGLEWMGALDPKTGDTAYSQKFKGRVTLTAD
ESTSTAYMELSSLRSEDTAVYYCTRFYSYTYWGQGTLVTVSSTTT
PAPRPPTPAPTIASQPLSLRPEACRPAAGGAVHTRGLDFACDIYI
WAPLAGTCGVLLLSLVITKRGRKKLLYIFKQPFMRPVQTTQEEDG
CSCRFPEEEEGGCELRVKFSRSADAPAYQQGQNQLYNELNLGRRE
EYDVLDKRRGRDPEMGGKPQRRKNPQEGLYNELQKDKMAEAYSEI
GMKGERRRGKGHDGLYQGLSTATKDTYDALHMQALPPR

In one embodiment, the GPC3 CAR comprises the amino acid sequence at least 90%, 91%, 92%, 93%, 94%, 95%, 96%, 97%, 98%, 99% sequence identity to the amino acid sequence:

(SEQ ID NO: 40)
MALPVTALLLPLALLLHAARPDVVMTQSPLSLPVTPGEPASISCR
SSQSLVHSNANTYLHWYLQKPGQSPQLLIYKVSNRFSGVPDRFSG
SGSGTDFTLKISRVEAEDVGVYYCSQNTHVPPTFGQGTKLEIKRG
STSGSGKPGSGEGSTKGQVQLVQSGAEVKKPGASVKVSCKASGYT
FTDYEMHWVRQAPGQGLEWMGALDPKTGDTAYSQKFKGRVTLTAD
ESTSTAYMELSSLRSEDTAVYYCTRFYSYTYWGQGTLVTVSSTTT
PAPRPPTPAPTIASQPLSLRPEACRPAAGGAVHTRGLDFACDIYI
WAPLAGTCGVLLLSLVITLYCKRGRKKLLYIFKQPFMRPVQTTQE
EDGCSCRFPEEEEGGCELRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPRGSGEG
RGSLLTCGDVEENPGPMAAPALSWRLPLLILLLPLATSWASAAVN
GTSQFTCFYNSRANISCVWSQDGALQDTSCQVHAWPDRRRWNQTC
ELLPVSQASWACNLILGAPDSQKLTTVDIVTLRVLCREGVRWRVM
AIQDFKPFENLRLMAPISLQVVHVETHRCNISWEISQASDFFERH
LEFEARTLSPGHTWEEAPLLTLKQKQEWICLETLTPDTQYEFQVR
VKPLQGEFTTWSPWSQPLAFRTKPAALGKDTIPWLGHLLVGLSGA
FGFIILVYLLINCRNTGPWLKKVLKCNTPDPSKFFSQLSSEHGGD
VQKWLSSPFPSSSFSPGGLAPEISPLEVLERDKVTQLLLQQDKVP
EPASLSSNHSLTSCFTNQGYFFFHLPDALEIEACQVYFTYDPYSE
EDPDEGVAGAPTGSSPQPLQPLSGEDDAYCTFPSRDDLLLFSPSL
LGGPSPPSTAPGGSGAGEERMPPSLQERVPRDWDPQPLGPPTPGV
PDLVDFQPPPELVLREAGEEVPDAGPREGVSFPWSRPPGQGEFRA
LNARLPLNTDAYLSLQELQGQDPTHLV

### B. Ortho CD122 Receptor

A second component of the ortho GPC3 CAR T cell is an ortho CD122 receptor. The terms “ortho CD122 receptor” or “ortho CD122” and “oCD122” and “hoRb” are used interchangeably to refer to a transmembrane polypeptide wherein the extracellular domain comprises the extracellular domain of human CD122 (hCD122) comprising or more amino acid substitutions at positions H133 and Y134 numbered in accordance with the mature human CD122 polypeptide and an intracellular domain capable of signaling in a mammalian immune cell. In some embodiments, the amino acid substitutions at positions H133 and Y134 are H133D and Y134F

In some embodiments, the ortho CD122 is a human CD122 mutein comprising amino substitutions at positions H133 and Y134 numbered in accordance with the mature human CD122 polypeptide. In some embodiments, the ortho CD122 is a human CD122 mutein comprising amino substitutions at positions H133D and Y134F numbered in accordance with the mature human CD122 polypeptide. In some embodiments, the ortho IL2 receptor has the amino acid sequence:

The term “ortho CD122 receptor” or “ortho CD122” includes chimeric orthogonal receptors wherein the naturally occurring intracellular domain of CD122 is replaced with a heterologous intracellular signaling domain As described in Garcia, et al., PCT International Publication Number WO 2021/050752, the intracellular domain of the orthogonal receptor may comprise the signaling domain of a receptor other that the intracellular domain of CD122. Orthogonal receptors that comprise an intracellular sequence heterologous to the CD122 ECD are referred to as chimeric ortho receptors. In one embodiment, when the membrane spanning receptor comprises an orthogonal CD122 ECD and CD122 ICD, the binding of an ortho IL2 to such receptor results in an intracellular signal characteristic of the activation of a Cd25/CD122/CD132 high affinity of CD122/CD132 intermediate affinity IL2 receptor. In an alternative embodiment, with respect to chimeric ortho receptors, the binding of ortho IL2 to the ECD of the chimeric ortho receptor results in intracellular signaling characteristic of the intracellular domain. For example, when the chimeric ortho receptor comprises the hoCD122 ECD and the intracellular domain is derived from the IL9 receptor, the intracellular signal induced by the binding of the hoIL2 to the chimeric receptor results in intracellular signaling characteristic of the IL9 receptor. Examples of ortho IL2 molecules are described Certain modified IL2 polypeptides are provided in Garcia, et al. (United States Patent Application Publication US2018/0228842A1 published Aug. 16, 2018 and Garcia, et al. U.S. Pat. No. 10,869,887 issued Dec. 22, 2020, the entire teachings of which are hereby incorporated by reference.

In one embodiment, the orthogonal CD122 is human CD122 comprising amino acid modifications at as positions 133 and 134 of numbered in accordance with the naturally occurring form of mature human CD122 (SEQ ID NO: 4). In some embodiments, the orthogonal CD122 is a hCD122 molecule comprising the amino acid substitutions H133D and Y134. In one embodiment, the orthogonal receptor is a modified human CD122 having the amino acid sequence (less the signal peptide) of the ECD of hCD122 having substitutions H133D and Y134F and the transmembrane (TM) and intracellular domain (ICD) of the wild-type hCD122 molecule having least 90%, 91%, 92%, 93%, 94%, 95%, 96%, 97%, 98%, 99% sequence identity to the amino acid sequence:

(SEQ ID NO: 6)
VNGTSQFTCFYNSRANISCVWSQDGALQDTSCQVHAWPDRRRWNQ
TCELLPVSQASWACNLILGAPDSQKLTTVDIVTLRVLCREGVRWR
VMAIQDFKPFENLRLMAPISLQVVHVETHRCNISWEISQASDFFE
RHLEFEARTLSPGHTWEEAPLLTLKQKQEWICLETLTPDTQYEFQ
VRVKPLQGEFTTWSPWSQPLAFRTKPAALGKDTIPWLGHLLVGLS
GAFGFIILVYLLINCRNTGPWLKKVLKCNTPDPSKFFSQLSSEHG
GDVQKWLSSPFPSSSFSPGGLAPEISPLEVLERDKVTQLLLQQDK
VPEPASLSSNHSLTSCFTNQGYFFFHLPDALEIEACQVYFTYDPY
SEEDPDEGVAGAPTGSSPQPLQPLSGEDDAYCTFPSRDDLLLFSP
SLLGGPSPPSTAPGGSGAGEERMPPSLQERVPRDWDPQPLGPPTP
GVPDLVDFQPPPELVLREAGEEVPDAGPREGVSFPWSRPPGQGEF
RALNARLPLNTDAYLSLQELQGQDPTHLV

In some embodiments the ICD of the orthogonal CD122 comprises, in addition to a native STAT5 recognition motif, one or more STAT3 binding motifs. The additional STAT3 binding motifs boosts the signaling and also stabilizes the IL2 response. STAT proteins act as transcriptional activators upon phosphorylation of a conserved tyrosine residue at the C terminus followed by translocation into the nucleus, where they bind to DNA and activate target gene transcription. Hennighausen and Robinson (2008) Genes Dev. 2008; 22:711-21. Seven STAT proteins have been identified in the STAT family: STATI, STAT2, STAT3, STAT4, STAT5A, STAT5B and STAT6, and they have functions in a variety of pathways, from innate and acquired immunity to cell proliferation, differentiation and survival. Basham et al., (2008) Nucleic Acids Res. 2008 June; 36 (11): 3802-3818. STATs binding motifs are typically present in cytokine receptors and binding of their respective cytokine ligand activates the tyrosine kinases in the Janus kinase (JAK) families, which phosphorylate certain tyrosine residues in the intracellular domains. The phosphorylated receptor recruits STATs to STAT recognition motifs on the receptor and the STAT becomes phosphorylated. The phosphorylated STATs dimerize and translocate to the nucleus wherein they initiate transcription of a variety of genes. Hennighausen, supra

In some embodiments, the modified orthogonal CD122 may comprise one, two, three, or more additional STAT3 binding motifs. In some embodiments, the STAT3 recognition motif has an amino acid sequence of YX₁×₂Q. In some embodiments, X₁ is selected from the group consisting of L, R, F, M, and X2 is selected from the group consisting of R, K, H, and P. In some embodiments, the STAT3 recognition motif has an amino acid sequence selected from the group consisting of: YLRQ; YLKQ; YRHQ; YLRQ; YFKQ; YLPQ; YMPQ, and YDKPH.

### CAR-T Cell Expression Vectors

The preparation of orthogonal GPC CAR T cells useful in the practice of the present invention is achieved by transforming isolated T cells with an expression vector comprising: (a) a nucleic acid sequence encoding the GPC CAR described above and (b) a nucleic acid sequence encoding an orthogonal receptor.

Expression vectors for expression of the CAR in the T-cell may be viral vectors or non-viral vectors. The term “nonviral vector” refers to an autonomously replicating, extrachromosomal circular DNA molecule, distinct from the normal genome and nonessential for cell survival under nonselective conditions capable of effecting the expression of a coding sequence in the target cell. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, magnetic fields (electroporation)

In some embodiments as exemplified herein, the expression vector is a viral vector. As used herein, the term viral vector is used in its conventional sense to refer to any of the obligate intracellular parasites having no protein-synthesizing or energy-generating mechanism and generally refers to any of the enveloped or non-enveloped animal viruses commonly employed to deliver exogenous transgenes to mammalian cells. A viral vector may be replication competent (e.g., substantially wild-type), conditionally replicating (recombinantly engineered to replicate under certain conditions) or replication deficient (substantially incapable of replication in the absence of a cell line capable of complementing the deleted functions of the virus). The viral vector can possess certain modifications to make it “selectively replicating,” i.e. that it replicates preferentially in certain cell types or phenotypic cell states, e.g., cancerous. Viral vector systems useful in the practice of the instant invention include, for example, naturally occurring or recombinant viral vector systems. Examples of viral vectors useful in the practice of the present disclosure include recombinantly modified enveloped or non-enveloped DNA and RNA viruses such as adenoviruses, vaccinia virus, lentivirus, retrovirusherpes virus, adeno-associated virus, human immunodeficiency virus, sindbis virus, and retroviruses (including but not limited to Rous sarcoma virus), and hepatitis B virus.

Typically, genes of interest are inserted into such vectors to allow packaging of the gene construct, typically with accompanying viral genomic sequences, followed by infection of a sensitive host cell resulting in expression of the gene of interest (e.g. a targeting antigen). Additionally, the expression vector encoding the GPC CAR may also be an mRNA vector. When a viral vector system is to be employed for transfection, retroviral or lentiviral expression vectors are preferred to transfect T-cells due to an enhanced efficacy of gene transfer to T-cells using these systems resulting in a decreased time for culture of significant quantities of T-cells for clinical applications. In particular, gamma retroviruses a particularly preferred for the genetic modification of clinical grade T-cells and have been shown to have therapeutic effect. Pule, et al. (2008) Nature Medicine 14 (11): 1264-1270. Similarly, self-inactivating lentiviral vectors are also useful as they have been demonstrated to integrate into quiescent T-cells. June, et al. (2009) Nat Rev Immunol 9 (10): 704-716.

The expression vector may encode one or more polypeptides in addition to the targeting antigen. When expressing multiple polypeptides as in the practice of the present invention, each polypeptide may be operably linked to an expression control sequence (monocistronic) or multiple polypeptides may be encoded by a polycistronic construct where multiple polypeptides are expressed under the control of a single expression control sequence.

Alternative to the use of multiple expression cassettes, the nucleic acid sequences encoding the CAR and IL-10 polypeptide may be encoded by a polycistronic construct, the expression cassette comprising the nucleic acid sequences the GPC3 CAR and the orthogonal receptor polypeptide employing an internal ribosome entry site (IRES) element or the foot and mouth disease virus protein 2A (FMVD2A) to facilitate co-expression in the target cell. In one embodiment of the disclosure, the nucleic acid sequence that facilitates co-expression comprises the nucleic acid sequence:

(SEQ ID NO: 41)
GGGAGTGGAGAGGGCCGCGGATCACTTCTCACATGCGGCGACGTA
GAGGAAAATCCTGGCCCC

Also referred to herein as T2A.

In one embodiment, the expression vector encoding the GPC3 CAR may optionally further encode one or more immunological modulators. Examples of immunological modulators useful in the practice of the present invention include but are not limited to cytokines. Examples of such cytokines are interleukins including but not limited to one more or of IL-1, IL-2, IL-3, IL-4, Il-10 IL-12, TNF-alpha, interferon alpha, interferon alpha-2b, interferon-beta, interferon-gamma, GM-CSF, MIP1-alpha, MIP1-beta, MIP3-alpha, TGF-beta and other suitable cytokines capable of modulating immune response. The expressed cytokines can be directed for intracellular expression or expressed with a signal sequence for extracellular presentation or secretion. IL-12 as reportedly resulted in enhanced antitumor efficacy (See Yeku, et al Scientific Reports Vol. 7, Article number: 10541 (2017) Published online: 5 Sep. 2017)

In one embodiment, the expression vector encoding the GPC3 CAR may optionally provide an additional expression cassette comprising a nucleic acid sequence encoding a “rescue” gene. A “rescue gene” is a nucleic acid sequence, the expression of which renders the cell susceptible to killing by external factors or causes a toxic condition in the cell such that the cell is killed. Providing a rescue gene enables selective cell killing of transduced cells. Thus the rescue gene provides an additional safety precaution when said constructs are incorporated into the cells of a mammalian subject to prevent undesirable spreading of transduced cells or the effects of replication competent vector systems. In one embodiment, the rescue gene is the thymidine kinase (TK) gene (see e.g. Woo, et al. U.S. Pat. No. 5,631,236 issued May 20, 1997 and Freeman, et al. U.S. Pat. No. 5,601,818 issued Feb. 11, 1997) in which the cells expressing the TK gene product are susceptible to selective killing by the administration of gancyclovir.

The expression vector may also include one or more selectable marker genes.

### Preparing Ortho Gpc3 T Cells

The present disclosure provides a method for the preparation of ortho GPC3 CAR T cells, the method comprising the steps of:
a. obtaining a population of T cells to be engineered;
b. optionally, enriching for one or more T cell subtypes;
c. transforming the T cells with a first nucleic acid sequence encoding orthogonal receptor and second nucleic acid sequence a GPC3-CAR wherein each the nucleic acid sequence is operably linked to an expression control sequence operable in a mammalian T cell such that the GPC3-CAR and orthogonal receptor are expressed on the surface of the T cell
d. contacting the population of T cells from step (c) with one or more agents that effect proliferation of the T cells.
   a. Obtaining a Population of T Cells to be Engineered

T cells useful in the preparation of a population of orthogonal GPC3-CAR T cells include naïve T-cells, central memory T-cells, effector memory T-cells or combinations thereof. T-cells may be obtained from the mammalian subject to be treated or may be any of a variety of T cell lines available in the art. T-cells for transformation are typically obtained from the mammalian subject to be treated. T cells can be obtained from a number of sources of the mammalian subject, including peripheral blood mononuclear cells, bone marrow, lymph node tissue, cord blood, thymus tissue, spleen tissue, and tumors. In one embodiment, T-cells are obtained by apheresis. In another embodiment, T cells are isolated from peripheral blood and particular T cells (such as CD3⁺, CD28⁺, CD4⁺, CD8⁺, CD45RA⁺, and CD45RO⁺ T cells) can be isolate by selection techniques well known in the art such is incubation with anti-CD3/anti-CD28 conjugated beads.

In some embodiments, T cell used for preparation of the orthogonal GPC3-CAR T cells modified to remove the endogenous TCRa and TCRb functions which are referred to as allogeneic T cells and the orthogonal GPC3-CAR T cells based on such. allogeneic T cells are referred to as allogeneic GPC3-CAR T cells. In some embodiments, the T cell to be used as the basis of the ortho GPC3-CAR T cells is be obtained from a subject to whom the orthogonal GPC3-CAR T cells derived therefrom are to be administered. In such instances, the orthogonal GPC3-CAR T cells are referred to as autologous orthogonal GPC3-CAR T cells. T cells for engineering as described above are collected from a subject or a donor may be separated from a mixture of cells by techniques that enrich for desired cells or may be engineered and cultured without separation.

b. Enriching for One or More T Cell Subtypes;

In some embodiments, the isolated population of T cells is enriched for certain T cell subtypes prior to transformation of the cells with the nucleic acid sequences encoding the GPC3-CAR and the orthogonal receptor. A variety of techniques are known in the art for the enrichment of a mixed population of T cells for particular T cell subtypes. Techniques providing accurate separation include fluorescence activated cell sorters. The cells may be selected against dead cells by employing dyes associated with dead cells (e.g., propidium iodide). The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification or may be frozen at liquid nitrogen temperatures and stored, being thawed and capable of being reused. The cells will usually be stored in 10% DMSO, 50% FCS, 40% RPMI 1640 medium. Ortho IL2 may be used to selectively expanding such ortho GPC3-CAR T cells in vitro or in vivo.

c. Transforming T Cells with Nucleic Acid Sequences Encoding a GPC3 CAR and an Orthogonal Receptor:

The population of selected T-cells is transformed with an expression vector encoding the GPC3-CAR in substantial accordance with teachings hereinabove.

d. Contacting the Population of T Cells Comprising a Mixed Population of Ortho GPC3 CAR T Cells with One or More Agents More Agents that Effect Proliferation of the T Cells.

Following transformation, T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005. Generally, the T cells of the invention are expanded by culturing the cells in contact with a surface providing an agent that stimulates a CD3 TCR complex associated signal (e.g., an anti-CD3 antibody) and an agent that stimulates a co-stimulatory molecule on the surface of the T cells (e.g an anti-CD28 antibody). Conditions appropriate for T cell culture are well known in the art Lin, et al. (2009) Cytotherapy 11 (7): 912-922 (Optimization and validation of a robust human T-cell culture method for monitoring phenotypic and polyfunctional antigen-specific CD4 and CD8 T-cell responses); Smith, et al. (2015) Clinical & Translational Immunology 4: e31 published online 16 Jan. 2015 (“Ex vivo expansion of human T cells for adoptive immunotherapy using the novel Xeno-free CTS Immune Cell Serum Replacement”). During the expansion phase, the population of T cells is maintained under conditions sufficient to support cell proliferation, for example, an appropriate temperature (e.g., 37° C.) and atmosphere (e.g., air plus 5% CO₂).

It will be apparent to those of skill in the art that the population of cells resulting from the foregoing step of contacting the cell population with a vector(s) encoding the GPC3-CAR and orthogonal ligand does not result in 100% efficiency of transduction such that the resulting cell population remains a mixed cell population comprising ortho GPC3-CAR T cells as well as non-transformed T cells. As it is desirable that the cell population for administration to a subject be as enriched as possible for the ortho GPC3-CAR T cells, in some the embodiments, during the expansion phase the cell population is contacted with an ortho IL2. Contacting a mixed cell population comprised of ortho GPC3-CAR T cells and non-transformed T cells with an ortho IL2 results in selective activation and proliferation of the ortho GPC3-CAR T cells such that the resulting cell product is substantially enriched for the presence of ortho GPC3-CAR T cells.

In some embodiments, the ortho IL2 used for the selective expansion of the GPC3-CAR T cells in the mixed cell population is an human IL2 mutein comprising the set of amino acid substitutions: E15S/H16Q/L19V/D20L/Q22K/M23A numbered in accordance with mature wild type human IL2.

In some embodiments, the ortho IL2 used for the selective expansion of the GPC3-CAR T cells in the mixed cell population is STK-007.

In some embodiments, the ortho IL2 used for the selective expansion of the GPC3-CAR T cells in the mixed cell population is STK-009.

In an alternative to transduction of the isolated T cell with a recombinant vector encoding the GPC3 CAR and ortho CD122 sequence, the nucleic acid sequence encoding the signal peptide-GPC3CAR-T2A-ortho CD122 polypeptide may be inserted into the genome of the T cell genome. In some embodiments, the present invention provides a ortho GPC3 CAR T cell wherein the nucleic acid sequences encoding the GPC3 CAR and ortho CD122 are inserted into the genome of a mammalian T cell. Homologous recombination was shown to promote the site-specific integration of large transgenes in the T cell genome (Schumann. et al. Generation of knock-in primary human T cells using Cas9 ribonucleoproteins. Proc Natl Acad Sci USA. (2015) 112:10437-42. doi: 10.1073/pnas. 1512503112). In this method, after the DNA of the target gene is cleaved using Cas9 RNPs, the gene of interest may be subsequently delivered to the cleavage site using adeno-associated viruses (AAVs). Site-specific transgene integration is achieved by HDR. An anti-CD19 CAR gene has been successfully integrated into the TRAC locus using the combined action of Cas9/RNP and AAV donor vectors (Eyquem, et al. Targeting a CAR to the TRAC locus with CRISPR/Cas9 enhances tumour rejection. Nature. (2017) 543:113-7. Targeting the nucleic acid sequence encoding the signal peptide-GPC3CAR-T2A-ortho CD122 polypeptide to the TRAC locus may provide only results in uniform expression GPC3 CAR and ortho CD122 polypeptides but also is reported to provide resistance to exhaustion of the CAR T cell. Furthermore, the insertion of a GPC3 CAR and ortho CD122 sequences into a defined position within the host cell genome avoids risks of inducing oncogenesis and places the GPC3 CAR and ortho CD122 expression under the control of endogenous regulatory elements.

The genomic insert of the nucleic acid sequences encoding the GPC3 CAR and ortho CD122 polypeptides may be achieve by sequence specific endonuclease that recognize and cleave the nucleic acid molecules a specific “target” sequences. Endonucleases are often categorized with respect to the degree of specificity and sequence identity characteristic of the target sequences. Some endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009) Nucleic Acids Research 37 (16): 5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23 (3): 967-973, a TALE-nuclease, a Cas9 endonuclease from CRISPR system as or a modified restriction endonuclease to extended sequence specificity (Eisenschmidt, et al. 2005; 33 (22): 7039-7047). In some embodiments of the invention, the immune cell (e.g. a CAR-T expressing the orthogonal receptor ECD of Formula 1) is modified to reduce alloreactivity through inactivation of one more components of the T-cell receptor (TCR). Methods for such modification of T cells is described in Galetto, et al. United States Patent Application Publication No. US 2013/015884A1 published Nov. 28, 2013 and methods for TCRalpha deficient T-cells by expressing pTalpha resulting in restoration of a functional CD3 complex as described in Galetto, et al. U.S. Pat. No. 10,426,795B2 issued Oct. 21, 2019. the teaching of which is herein incorporated by reference.

### Ortho IL2:

As used herein, the term “ortho IL2” or “oIL2” refers to a IL2 mutein derived from an IL2 parent polypeptide wherein the ortho IL2 specifically binds to the extracellular domain of ortho CD122 receptor and exhibits significantly reduced binding to the extracellular domain of a wild type CD122. In some embodiment the ortho IL2 exhibits specific binding to a receptor comprising an orthogonal CD122 ECD and (2) the contacting of a cell expressing a membrane spanning receptor comprising the ECD of an orthogonal CD122 polypeptide in an amount sufficient to cause a response results in the a signal characteristic of the signal produced by the intracellular domain (ICD) of said membrane spanning receptor.

As described in Garcia, et al., PCT International Publication Number WO 2021/050752, the intracellular domain of the orthogonal receptor may comprise the signaling domain of a receptor other that the intracellular domain of CD122. Orthogonal receptors that comprise an intracellular sequence heterologous to the CD122 ECD are referred to as chimeric ortho receptors. In one embodiment, when the membrane spanning receptor comprises an orthogonal CD122 ECD and CD122 ICD, the binding of an ortho IL2 to such receptor results in an intracellular signal characteristic of the activation of a Cd25/CD122/CD132 high affinity of CD122/CD132 intermediate affinity IL2 receptor. In an alternative embodiment, with respect to chimeric ortho receptors, the binding of ortho IL2 to the ECD of the chimeric ortho receptor results in intracellular signaling characteristic of the intracellular domain. For example, when the chimeric ortho receptor comprises the hoCD122 ECD and the intracellular domain is derived from the IL9 receptor, the intracellular signal induced by the binding of the hoIL2 to the chimeric receptor results in intracellular signaling characteristic of the IL9 receptor. Examples of ortho IL2 molecules are described Certain modified IL2 polypeptides are provided in Garcia, et al. (United States Patent Application Publication US2018/0228842A1 published Aug. 16, 2018 and Garcia, et al. U.S. Pat. No. 10,869,887 issued Dec. 22, 2020, the entire teachings of which are hereby incorporated by reference.

In some embodiments, the ortho hIL2 the amino acid sequence of which has at least least 90%, alternatively at least 91%, alternatively at least 92%, alternatively at least 93%, alternatively at least 94%, alternatively at least 95%, alternatively at least 96%, alternatively at least 97%, alternatively at least 98%, or alternatively at least 99% sequence identity to any one of identity to a polypeptide having the amino acid sequence:

(AA1)-(AA2)-(AA3)-(AA4)-(AA5)-(AA6)-(AA7)-
(AA8)-(AA9)ᵢ-T10-Q11-L12-(AA13)-(AA14)-(AA15)-
(AA16)-L17-(AA18)-(AA19)-(AA20)-L21-(AA22)-
(AA23)-I24-L25-N26-(AA27)-I28-N29-N30-Y31-K32-
N33-P34-K35-L36-T37-(AA38)-(AA39)-L40-T41-
(AA42)-K43-F44-Y45-M46-P47-K48-K49-A50-(AA51)-
E52-L53-K54-(AA55)-L56-Q57-C58-L59-E60-E61-
E62-L63-K64-P65-L66-E67-E68-V69-L70-N71-L72-
A73-(AA74)-S75-K76-N77-F78-H79-(AA80-(AA81)-
P82-R83-D84-(AA85)-(AA86)-S87-N88-(AA89)-N90-
(AA91)-(AA92)-V93-L94-E95-L96-(AA97)-G98-S99-
E100-T101-T102-F103-(AA104)-C105-E106-Y107-
A108-(AA109)-E110-T111-A112-(AA113)-I114-
V115-E116-F117-L118-N119-R120-W121-I122-T123-
F124-(AA125)-(AA126)-S127-I128-I129-(AA130)-
T131-L132-T133

wherein:
   AA1 is A (wild type) or deleted;
   AA2 is P (wild type) or deleted;
   AA3 is T (wild type), C, A, G, Q, E, N, D, R, K, P, or deleted;
   AA4 is S (wild type) or deleted;
   AA5 is S (wild type) or deleted;
   AA6 is S (wild type) or deleted;
   AA7 is T (wild type) or deleted;
   AA8 is K (wild type) or deleted;
   AA9 is K (wild type) or deleted;
   AA13 is Q (wild type), W or deleted;
   AA14 is L (wild type), M, W or deleted;
   AA15 is E (wildtype), K, D, T, A, S, Q, H or deleted;
   AA16 is H (wildtype), N or Q or deleted;
   AA18 is L (wild type) or R, L, G, M, F, E, H, W, K, Q, S, V, I, Y, H, D or T;
   AA19 is L (wildtype), A, V, I or deleted;
   AA20 is D (wildtype), T, S M L, or deleted;
   AA22 is Q (wild type) or F, E, G, A, L, M, F, W, K, S, V, I, Y, H, R, N, D, T, F or deleted;
   AA23 is M (wild type), A, W, H, Y, F, Q, S, V, L, T, or deleted;
   AA27 IS G (wildtype), K, S or deleted;
   AA38 is R (wild type), W or G;
   AA39 is M (wildtype), L or V;
   AA42 is F (wildtype) or K;
   AA51 is T (wildtype), I or deleted
   AA55 is H (wildtype) or Y;
   AA74 is Q (wild type), N, H, S;
   AA80 is L (wild type), F or V;
   AA81 is R (wild type), I, D, Y, T or deleted
   AA85 is L (wild type) or V;
   AA86 is I (wild type) or V;
   AA88 is N (wildtype), E or Q or deleted;
   AA89 is I (wild type) or V;
   AA91 is V (wild type), R or K;
   AA92 is I (wild type) or F;
   AA97 is K (wild type) or Q;
   AA104 is M (wild type) or A;
   AA109 is D (wildtype), C or a non-natural amino acid with an activated side chain;
   AA113 is T (wild type) or N;
   AA125 is C (wild type), A or S;
   AA126 is Q (wild type) or H, M, K, C, D, E, G, I, R, S, or T; and/or
   AA130 is S (wild type), T or R.

In one embodiment, the, the ortho IL2 is a human ortho IL2 comprising the set of amino acid substitutions: E15S/H16Q/L19V/D20L/Q22K/M23A numbered in accordance with the mature human IL2 (SEQ ID NO:7)

In one embodiment, the ortho IL2 is a human ortho IL2 comprising the set of amino acid substitutions: desAla1/E15S-H16Q-L19V-D20L-Q22K-M23A].

In one embodiment, the ortho IL2 is a polypeptide comprising the amino acid sequence:

(SEQ ID NO: 9)
PTSSSTKKTQLQLSQLLVLLKAILNGINNYKNPKLTRMLTFKFYM
PKKATELKHLQCLEEELKPLEEVLNLAQSKNFHLRPRDLISNINV
IVLELKGSETTFMCEYADETATIVEFLNRWITFCQSIISTLT

Also referred to as STK-007.

In some embodiments, the present disclosure provides human ortho IL2s to facilitate recombinant expression in bacterial cells by eliminating the unpaired cysteine residue at position 125 and/or elimination of the N-terminal Met of the directly expressed IL2 polypeptide as well as the alanine at position 1 by post-translational processing by endogenous bacterial proteases. When an amino acid is missing, it is referred to as “des”. In some embodiments, the cysteine at position 125 is substituted with alanine or serine (C125A or C125S). Such mutations are typically used to avoid misfolding of the protein when expressed recombinantly in bacteria and isolated from inclusion bodies.

In some embodiments, human ortho IL2s contain one or more mutations in positions of the hIL2 sequence that either contact hCD122 or alter the orientation of other positions contacting CD122, resulting in an ortho IL2 having increased affinity for CD122. IL2 residues that have been identified as being involved in the binding of IL2 to CD122 include L12, Q13, H16, L19, D20, M23, Q74, L80, R81, D84, L85, I86, S87, N88, I89 V91, I92, and E95. In some embodiments, the ortho IL2 comprises one or more of the amino acid substitutions: Q74N, Q74H, Q74S, L80F, L80V, R81D, R81T, L85V, I86V, I89V, and/or I92F or combinations thereof. In some embodiments, the ortho IL2 comprises one or more of the amino acid substitutions: L80F, R81D, L85V, I86V and I92F. In some embodiments, the ortho IL2 comprises one or more of the amino acid substitutions: N74Q, L80F, R81D, L85V, I86V, 189V, and I92F. In some embodiments, the ortho IL2 comprises one or more of the amino acid substitutions: Q74N, L80V, R81T, L85V, I86V, and I92F. In some embodiments, the ortho IL2 comprises one or more of the amino acid substitutions: Q74H, L80F, R81D, L85V, I86V and I92F. In some embodiments, the ortho IL2 comprises one or more of the amino acid substitutions: Q74S, L80F, R81D, L85V, I86V and I92F. In some embodiments, the ortho IL2 comprises one or more of the amino acid substitutions: Q74N, L80F, R81D, L85V, I86V and I92F. In some embodiments, the ortho IL2 comprises one or more of the amino acid substitutions: Q74S, R81T, L85V, and I92F. In some embodiments, the ortho IL2 comprises [L80F-R81D-L85V-I86V-I92F]. In some embodiments, the ortho IL2s comprise the substitution L85V that has been identified as increasing affinity of IL2 to CD122.

### Modifications to Modulate CD25 Affinity

In some embodiments, the ortho IL2s contain one or more mutations in positions of the IL2 sequence that either contact CD25 or alter the orientation of other positions contacting CD25 resulting in a decreased affinity for CD25. The mutations may be in or near areas known to be in close proximity to CD25 based on published crystal structures (Wang, et al Science 310:1159 2005). IL2 residues believed to contact CD25 include K35, R38, T41, F42, K43, F44, Y45, E61, E62, K64, P65, E68, V69, L72, and Y107. In some embodiments, the ortho IL2s of the present disclosure comprise one or more of the point mutations of R38A, F41A and F42A (Suave, et al (1991) PNAS (USA) 88:4636-4640); P65L (Chen et al. Cell Death and Disease (2018) 9:989); F42A/G/S/T/Q/E/N/R/K, Y45A/G/S/T/Q/E/N/D/R/K/and/or L72G/A/S/T/Q/E/N/D/R/K (Ast, et al United States Patent Application Publication 2012/0244112A1 published Sep. 27, 2012; U.S. Pat. No. 9,266,938B2 issued Feb. 23, 2016). Particular combinations of substitutions have been identified as reducing binding to CD25. In some embodiments, the ortho IL2s of the present disclosure comprise one or more of the of the sets of substitutions [R38A-F42A-Y45A-E62A] as described in Carmenate, et al (2013) J Immunol 190:6230-6238; [F42A-Y45A-L72G] (Roche RG7461 (RO6874281); and/or [T41P-T51P] (Chang, et al (1995) Molecular Pharmacology 47:206-211).

### Modifications to Modulate CD132 Affinity

In some embodiments of the invention, the ortho IL2s contain one or more mutations in positions of the IL2 sequence that either contact CD132 or alter the orientation of other positions contacting CD132 resulting in an altered binding to CD132. Exemplary ortho IL2s contain one or more mutations in positions of the IL2 sequence that either contact CD132 or alter the orientation of other positions contacting CD122, resulting in an altered binding to CD132. IL2 residues believed to contact CD132 include Q11, L18, Q22, E110, N119, T123, Q126, S127, I129, S130, and T133. In some embodiments, the IL2 comprises modifications at L18 AA18 is L (wild type) or R, L, G, M, F, E, H, W, K, Q, S, V, I, Y, H, D or T; AA126 is Q (wild type) or H, M, K, C, D, E, G, I, R, S, or T; and/or AA22 is Q (wild type) or F, E, G, A, L, M, F, W, K, S, V, I, Y, H, R, N, D, T, or F.

When produced recombinantly in bacterial expression systems directly in the absence of a leader sequence, endogenous proteases result in the deletion of the N-terminal Met-Alal residues to provide “desAla1” ortho IL2s. In some embodiments, the present disclosure provides human ortho IL2s which are hIL2 polypeptides comprising one of the following sets of amino acid modifications:

The ortho IL2s of the present disclosure may comprises comprise modifications to eliminate the O-glycosylation site at position Thr3 to facilitate the production of an aglycosylated ortho IL2 when the ortho IL2 expressed in mammalian cells such as CHO or HEK cells. Thus, in certain embodiments the ortho IL2 comprise a modification which eliminates the O-glycosylation site of IL-2 at a position corresponding to residue 3 of human IL-2. In one embodiment said modification which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P which removes the glycosylation site at position 3 without eliminating biological activity (see U.S. Pat. No. 5,116,943; Weiger et al., (1989) Eur. J. Biochem., 180:295-300). In some embodiments the ortho IL2 may comprise deletion of the first two amino acids (desAla1-desPro2) as well as substitution of the Thr3 glycosylation with a cysteine residue to facilitate for selective N-terminal modification, especially PEGylation of the sulfhydryl group of the cysteine (See, e.g. Katre, et al. U.S. Pat. No. 5,206,344 issued Apr. 27, 1993).

The ortho IL2s may optionally further comprise a modification at position M104, in one embodiment the substitution of methionine 104 with an alanine residue (M104A) to provide a more oxidation-resistant ortho IL2 (See Koths, et al. U.S. Pat. No. 4,752,585.

When produced recombinantly in bacterial expression systems directly in the absence of a leader sequence, endogenous proteases result in the deletion of the N-terminal Met-Alal residues to provide “desAla1” ortho IL2s. ortho IL2s may comprise deletion of the first two amino acids (desAla1-desPro2) as well as substitution of the Thr3 glycosylation with a cysteine residue (T3C) to facilitate for N-terminal modification, especially PEGylation of the sulfhydryl group of the cysteine (See, e.g. Katre, et al. U.S. Pat. No. 5,206,344 issued Apr. 27, 1993).

The ortho IL2s may further comprise elimination of N-terminal amino acids at one or more of positions 1-9, alternatively positions 1-8, alternatively positions 1-7 alternatively positions 1-6, alternatively positions 1-5, alternatively positions 1-4, alternatively positions 1-3, alternatively positions 1-2.

In some embodiments of the disclosure, the ortho IL2 comprises amino acid substitutions to avoid vascular leak syndrome, a substantial negative and dose limiting side effect of the use of IL2 therapy in human beings without out substantial loss of efficacy. See, Epstein, et al., U.S. Pat. No. 7,514,073B2 issued Apr. 7, 2009. Examples of such modifications which are included in the ortho IL2s of the present disclosure include one or more of R38W, R38G, R39L, R39V, F42K, and H55Y.

In some embodiments, ortho IL2s may be affinity matured to enhance their activity with respect to the orthogonal CD122. An “affinity matured” polypeptide is one having one or more alteration(s) in one or more residues which results in an improvement in the affinity of the orthogonal polypeptide for the cognate orthogonal receptor, or vice versa, compared to a parent polypeptide which does not possess those alteration(s). Affinity maturation can be done to increase the binding affinity of the ortho IL2 by at least about 10%, alternatively at least about 50%, alternatively at least about 100% alternatively at least about 150%, or from 1 to 5-fold as compared to the “parent” polypeptide. An engineered ortho IL2 of the present invention activates its cognate orthogonal receptor, as discussed above, but has significantly reduced binding and activation of the wild-type IL2 receptor when assessed by ELISA and/or FACS analysis using sufficient amounts of the molecules under suitable assay conditions.

As discussed above, the compositions of the present disclosure include ortho IL2s that have been modified to provide for an extended lifetime in vivo and/or extended duration of action in a subject. Such modifications to provided extended lifetime and/or duration of action include modifications to the primary sequence of the ortho IL2, conjugation to carrier molecules, (e.g. albumin, acylation, PEGylation), and Fc fusions.

In some embodiments, the ortho IL2 may comprise certain amino acid substitutions that result in prolonged in vivo lifetime. For example, Dakshinamurthi, et al. (International Journal of Bioinformatics Research (2009) 1 (2): 4-13) state that one or more of the substitutions in the IL2 polypeptide V91R, K97E and T113N will result in an IL2 variant possessing enhanced stability and activity. In some embodiments, the ortho IL2s of the present disclosure comprise one, two or all three of the V91R, K97E and T113N modifications.

In some embodiments the ortho IL2 is modified to provide certain properties to the ortho IL2 (e.g. extended duration of action in a subject) which may be achieve through conjugation to carrier molecules to provide desired pharmacological properties such as extended half-life. In some embodiments, the ortho IL2 can be covalently linked to the Fc domain of IgG, albumin, or other molecules to extend its half-life, e.g. by PEGylation, glycosylation, fatty acid acylation, and the like as known in the art.

In some embodiments, the ortho IL2 is expressed as a fusion protein with an albumin molecule (e.g. human serum albumin) which is known in the art to facilitate extended exposure in vivo.

In one embodiment of the invention, the human ortho IL2 is conjugated to albumin referred to herein as an “ortho IL2 albumin fusion.” The term “albumin” as used in the context human ortho IL2 albumin fusions include albumins such as human serum albumin (HSA), cyno serum albumin, and bovine serum albumin (BSA). In some embodiments, the HSA the HSA comprises a C34S or K573P amino acid substitution relative to the wild type HSA sequence According to the present disclosure, albumin can be conjugated to a human ortho IL2 at the carboxyl terminus, the amino terminus, both the carboxyl and amino termini, and internally (see, e.g., U.S. Pat. Nos. 5,876,969 and 7,056,701). In the HSA-human ortho IL2 polypeptide conjugates contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a human ortho IL2 polypeptide fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker such as a peptide linker or modified version thereof as more fully discussed below.

Alternatively, the human ortho IL2 albumin fusion comprises ortho IL2s that are fusion proteins which comprise an albumin binding domain (ABD) polypeptide sequence and an ortho IL2 polypeptide. As alluded to above, fusion proteins which comprise an albumin binding domain (ABD) polypeptide sequence and a human ortho IL2 polypeptide can, for example, be achieved by genetic manipulation, such that the nucleic acid coding for HSA, or a fragment thereof, is joined to the nucleic acid coding for the one or more ortho IL2 sequences. In some embodiments, the albumin-binding peptide comprises the amino acid sequence DICLPRWGCLW (SEQ ID NO:50).

The ortho IL2 polypeptide can also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria, dendritic cells, thyroglobulin; tetanus toxoid; Diphtheria toxoid; polyamino acids such as poly (D-lysine: D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen Such conjugated forms, if desired, can be used to produce antibodies against a polypeptide of the present disclosure.

In some embodiments, the ortho IL2 is conjugated (either chemically or as a fusion protein) with an XTEN which provides extended duration of akin to PEGylation and may be produced as a recombinant fusion protein in E. coli. XTEN polymers suitable for use in conjunction with the ortho IL2s of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE|DOI: 10.1371/journal.pone. 0157193 Jun. 13, 2016. The XTEN polymer fusion protein may incorporate a protease sensitive cleavage site between the XTEN polypeptide and the ortho IL2 such as an MMP-2 cleavage site.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

In some embodiments, the IL-2 mutein also may be linked to additional therapeutic agents including therapeutic compounds such as anti-inflammatory compounds or antineoplastic agents, therapeutic antibodies (e.g. Herceptin), immune checkpoint modulators, immune checkpoint inhibitors (e.g. anti-PD1 antibodies), cancer vaccines as described elsewhere in this disclosure. Anti-microbial agents include aminoglycosides including gentamicin, antiviral compounds such as rifampicin, 3′-azido-3′-deoxythymidine (AZT) and acylovir, antifungal agents such as azoles including fluconazole, plyre macrolides such as amphotericin B, and candicidin, anti-parasitic compounds such as antimonials, and the like. The ortho IL2 may be conjugated to additional cytokines as CSF, GSF, GMCSF, TNF, erythropoietin, immunomodulators or cytokines such as the interferons or interleukins, a neuropeptide, reproductive hormones such as HGH, FSH, or LH, thyroid hormone, neurotransmitters such as acetylcholine, hormone receptors such as the estrogen receptor. Also included are non-steroidal anti-inflammatoires such as indomethacin, salicylic acid acetate, ibuprofen, sulindac, piroxicam, and naproxen, and anesthetics or analgesics. Also included are radioisotopes such as those useful for imaging as well as for therapy.

The ortho IL2s of the present disclosure may be chemically conjugated to such carrier molecules using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to IL-2 mutein and can readily be identified by those skilled in the art. Alternatively, or in addition, the ortho IL2 and/or the molecule to which it is intended to be conjugated may be chemically derivatized such that the two can be conjugated in a separate reaction as is also well known in the art.

In some embodiments, the ortho IL2 is conjugated to one or more water-soluble polymers. Examples of water soluble polymers useful in the practice of the present invention include polyethylene glycol (PEG), poly-propylene glycol (PPG), polysaccharides (polyvinylpyrrolidone, copolymers of ethylene glycol and propylene glycol, poly (oxyethylated polyol), polyolefinic alcohol, polysaccharides, poly-alpha-hydroxy acid, polyvinyl alcohol (PVA), polyphosphazene, polyoxazolines (POZ), poly (N-acryloylmorpholine), or a combination thereof.

In some embodiments the ortho IL2 is conjugated to one or more polyethylene glycol molecules or “PEGylated.” Although the method or site of PEG attachment to ortho IL2 may vary, in certain embodiments the PEGylation does not alter, or only minimally alters, the activity of the ortho IL2.

In some embodiments, a cysteine may be substituted for the threonine at position 3 (3TC) to facilitate N-terminal PEGylation using particular chemistries.

In some embodiments, selective PEGylation of the ortho IL2 (for example by the incorporation of non-natural amino acids having side chains to facilitate selective PEG conjugation chemistries as described Ptacin, et al., (PCT International Application No. PCT/US2018/045257 filed Aug. 3, 2018 and published Feb. 7, 2019 as International Publication Number WO 2019/028419A1 may be employed to generate an ortho IL2 with having reduced affinity for one or more subunits (e.g. CD25, CD132) of an IL2 receptor complex. For example, an human ortho IL2 incorporating non-natural amino acids having a PEGylatable specific moiety at those sequences or residues of IL2 identified as interacting with CD25 including amino acids 34-45, 61-72 and 10⁵-109 typically provides an ortho IL2 having diminished binding to CD25. Similarly, a human ortho IL2 incorporating non-natural amino acids having a PEGylatable specific moiety at those sequences or residues of IL2 identified as interacting with hCD132 including amino acids 18, 22, 109, 126, or from 119-133 provides an ortho IL2 having diminished binding to hCD132.

In certain embodiments, the increase in half-life is greater than any decrease in biological activity. PEGs suitable for conjugation to a polypeptide sequence are generally soluble in water at room temperature and have the general formula R (O-CH2-CH2) nO-R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the PEG-ortho IL2 can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, alternatively about 30,000 to about 40,000 daltons. In one embodiment of the invention, the PEG is a 40 kD branched PEG comprising two 20 kD arms.

The present disclosure also contemplates compositions of conjugates wherein the PEGs have different n values, and thus the various PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R (O-CH2-CH2) nO-R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein.

The PEG can be bound to an ortho IL2 of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of ortho IL2s is facilitated by the incorporation of non-natural amino acids bearing unique side chains to facilitate site specific PEGylation. The incorporation of non-natural amino acids into polypeptides to provide functional moieties to achieve site specific pegylation of such polypeptides is known in the art. See e.g. Ptacin, et al., (PCT International Application No. PCT/US2018/045257 filed Aug. 3, 2018 and published Feb. 7, 2019 as International Publication Number WO 2019/028419A1. In one embodiment, the ortho IL2s of the present invention incorporate a non-natural amino acid at position D109 of the ortho IL2. In one embodiment of the invention the ortho IL2 is a PEGylated at position 109 of the ortho IL2 to a PEG molecule having a molecular weight of about 20 kD, alternatively about 30 kD, alternatively about 40 kD.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present invention include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g. Sunbright® ME-200AL, NOF, a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

As previously noted, the PEG may be attached directly to the ortho IL2 or via a linker molecule. Suitable linkers include “flexible linkers” which are generally of sufficient length to permit some movement between the modified polypeptide sequences and the linked components and molecules. The linker molecules are generally about 6-50 atoms long. The linker molecules can also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers include glycine polymers (G) n, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G) n, glycine-alanine polymers, alanine-serine polymers, glycine-serine polymers. Glycine and glycine-serine polymers are relatively unstructured, and therefore may serve as a neutral tether between components. A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein.

Further, such linkers may be used to link the ortho IL2 to additional heterologous polypeptide components as described herein, the heterologous amino acid sequence may be a signal sequence and/or a fusion partner, such as, albumin, Fc sequence, and the like.

In one embodiment of the disclosure, the ortho IL2 is a human ortho IL2 of the structure:

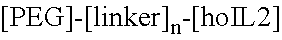

wherein n=0 or 1, or

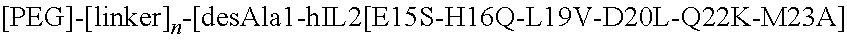

wherein n=0 or 1, or

In another embodiment of the invention, the ortho IL2 is a human ortho IL2 of the structure

40kD-PEG-(linker)ₙ-PTSSSTKKTQLQLSQLLVLLKAILNGINNYK
NPKLTRMLTFKFYMPKKATELKHLQCLEEELKPLEEVLNLAQSKNFHLRP
RDLISNINVIVLELKGSETTFMCEYADETATIVEFLNRWITFCQSIISTL
T

wherein n=0 (absent) or 1 (present).

In one embodiment the ortho IL2 is a polypeptide of the

40kD-PEG-(linker)ₙ-PTSSSTKKTQLQLSQLLVLLKAILNGINNYK
NPKLTRMLTFKFYMPKKATELKHLQCLEEELKPLEEVLNLAQSKNFHLRP
RDLISNINVIVLELKGSETTFMCEYADETATIVEFLNRWITFCQSIISTL
T

wherein the 40 kD-PEG-Linker (n=1) is a molecule of the structure:

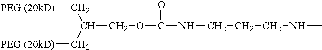

also referred to herein as STK-009

In some embodiments, the ortho IL2 is provided as a fusion protein with a polypeptide sequence (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between the ortho IL2 sequence and the sequence of the targeting domain of the fusion protein.

In other embodiments, a chimeric polypeptide including a orthogonal IL-2 and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting domain of the ortho IL2 fusion protein specifically binds to a cell surface molecule of a tumor cell. In one embodiment wherein the ECD of the CAR of a CAR-T cell specifically binds to GPC3, the ortho IL2 may be provided as a fusion protein with a GPC3 targeting moiety. For example, in one embodiment wherein the ECD of the CAR of a CAR-T cell is an scFv molecule that provides specific binding to GPC3, the ortho IL2 is provided as a fusion protein with a GPC3 targeting moiety such as a single chain antibody (e.g., an scFv or VHH) that specifically binds to GPC3. In some embodiments, targeting domain is an scFv derived from GC33. In some embodiments, the targeting domains is an scFv selected from SEQ ID NO:10 or SEQ ID NO:11.

### Therapeutic Methods

The present disclosure provides methods and compositions for treating a subject suffering from a neoplastic disease.

In one embodiment the present invention provides a method treating or preventing a neoplastic disease, disorder, or condition in a mammalian subject in need of treatment or prevention, the method comprising the steps of:
(a) isolating a population of T cells from the subject;
(b) contacting said population of T cells with recombinant viral vector, the vector comprising a first nucleic acid sequence encoding a GPC3 CAR and a second nucleic acid sequence encoding an orthogonal receptor, said first and second nucleic acid sequences are operably linked to an expression control element functional in a mammalian T cell, the contacting resulting in a fraction of the isolated population of T cells is transformed by the viral vector such that the transformed cell expresses the GPC3 CAR and the orthogonal receptor;
(c) administering to the subject a therapeutically effective amount the T cells resulting from step (b) in combination with the administration to the subject of a therapeutically effective amount of an ortho IL2.

In one embodiment the present invention provides a method of treatment of a subject suffering from a neoplastic disease the method comprising the administering to the subject of a therapeutically effective quantity of ortho GPC3 CAR T cells in combination with a therapeutically effective amount of ortho IL2. In some embodiments, the antigen binding domain of the GPC3 CAR cell is an scFv. In some embodiments, the scFv is a GC33 scFv. In some embodiments, the scFv is SEQ ID NO: 10 or SEQ ID NO: 11.

In some embodiments, the intracellular domain of the GPC3 CAR T comprises a CD28 costimulatory domain and a CD3-zeta domain. In some embodiments, the intracellular domain of the GPC3 CAR T comprises a 41BB costimulatory domain and a CD3-zeta domain.

In some embodiments, the GPC3 CAR is a polypeptide having at least 90%, alternatively at least 91%, alternatively at least 92%, alternatively at least 93%, alternatively at least 94%, alternatively at least 95%, alternatively at least 96%, alternatively at least 97%, alternatively at least 98%, or alternatively at least 99% sequence identity to any one of SEQ ID NOS: 34, 35, 36, 37, 38, 39 and 40. In some embodiments, the GPC3 CAR is selected from the group consisting of SEQ ID NOS: 34, 35, 36, 37, 38, 39 and 40.

In some embodiments the neoplastic disease is selected from hepatocellular carcinoma, hepatoblastoma, lung squamous cell carcinoma, ovarian yolk sac tumor, melanoma and clear cell carcinoma of the ovary.

In some embodiments the present disclosure provides a method of treatment of a subject suffering from a neoplastic disease characterized by the presence in the subject of a neoplasm that expresses GPC3 (“GPC3+ neoplasms”).

As used herein the term “subject” is used interchangeably with the terms “recipient”, “individual”, and “patient”, refer to any mammalian subject for whom diagnosis, treatment, or therapy is desired. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm. As previously discussed above, the presence of GPC3+ neoplasms may be identified by the presence of the presence of the 40 kDa subunit of GPC3 referred to as “soluble GPC3” or “sGPC3” which is detectable in the serum and the level of GPC3 is suggested as a diagnostic marker.

As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions.

The terms “administration” and “administer” are used interchangeably herein to refer the act of contacting a subject, including contacting a cell, tissue, organ, or biological fluid of the subject in vitro, in vivo or ex vivo with an agent (e.g. an ortholog, an IL2 ortholog, an engineered cell expressing an orthogonal receptor, an engineered cell expressing an orthogonal IL2 receptor (e.g. a CAR-T cell expressing an orthogonal IL2 receptor) a chemotherapeutic agent, an antibody) or a pharmaceutical formulation comprising one or more of the foregoing. Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), inhalation (e.g respiratory inhalers including dry-powder inhalers), intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell, tissue or organ.

The term “therapeutically effective amount” as used herein in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15 (23): 7412-7420. A therapeutically effective amount may be adjusted over a course of treatment of a subject in connection with the dosing regimen and/or evaluation of the subject's condition and variations in the foregoing factors. In one embodiment, a therapeutically effective amount is an amount of an agent when used alone or in combination with another agent does not result in non-reversible serious adverse events in the course of administration to a mammalian subject.

As used herein, the term “in combination with” when used in reference to the administration of multiple agents to a subject refers to the administration of a first agent at least one additional (i.e. second, third, fourth, fifth, etc.) agent to a subject. For purposes of the present invention, one agent (e.g. IL2 ortholog) is considered to be administered in combination with a second agent (e.g. an engineered human immune cell) if the biological effect resulting from the administration of the first agent persists in the subject at the time of administration of the second agent such that the therapeutic effects of the first agent and second agent overlap. For example, an engineered orthogonal cell therapy agent would typically be administered infrequently (typically only a single administration) while the while the IL2 orthologs are administered periodically while the orthogonal cell agent persists in the subject. The engineered orthogonal cell therapy agent provides a therapeutic effect over an extended time (weeks or months) and the administration of the second agent (e.g. an IL2 ortholog) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject.

In some embodiments, the ortho IL2 is administered in combination with the ortho GPC3 CAR T cells, wherein the ortho IL2 is administered to the subject contemporaneously with administration of the ortho GPC3 CAR T cells to the subject.

In some embodiments, the ortho IL2 is administered in combination with the ortho GPC3 CAR T cells, wherein the ortho IL2 is administered to the subject at one or more time points 1, 2, 3, 4, 5, 6, 7, 8, 9, 10, 11, 12, 13 or 14 days prior to the administration of the ortho GPC3 CAR T cells to the subject.

In some embodiments, the ortho IL2 is administered in combination with the ortho GPC3 CAR T cells, wherein the ortho IL2 is administered to the subject at one or more time points 1, 2, 3, 4, 5, 6, 7, 8, 9, 10, 11, 12, 13, 14, 20, 30, 60, 90, 120, 150, 180, 240 days after the administration of the ortho GPC3 CAR T cells to the subject.

In some embodiments, the ortho IL2 is administered in combination with the ortho GPC3 CAR T cells, wherein the ortho IL2 is administered (a) 1, 2, 3, 4, 5, 6, 7, 8, 9, 10, 11, 12, 13 or 14 days prior to the administration of the ortho GPC3 CAR T cells to the subject; (b) contemporaneously with administration of the ortho GPC3 CAR T cells to the subject, and (c) at one or more time points 1, 2, 3, 4, 5, 6, 7, 8, 9, 10, 11, 12, 13, 14, 20, 30, 60, 90, 120, 150, 180, 240 days after the administration of the ortho GPC3 CAR T cells to the subject.

In some embodiments, the ortho IL2 is administered in combination with the ortho GPC3 CAR T cells, wherein the ortho IL2 is administered (a) contemporaneously with administration of the ortho GPC3 CAR T cells to the subject and (b) at one or more time points 1, 2, 3, 4, 5, 6, 7, 8, 9, 10, 11, 12, 13, 14, 20, 30, 60, 90, 120, 150, 180, 240 days after the administration of the ortho GPC3 CAR T cells to the subject.

In certain embodiments, orthogonal cell and IL2 ortholog is further combined in combination with supplementary anti-neoplastic agents. The supplementary agent(s) may be administered prior to, during and/or following the a course of treatment with the GPC3 CAR T cells in combination ortho IL2. In some embodiments, the supplementary anti-neoplastic agent is selected from the group consisting of chemotherapeutic agents, small molecules, supplementary biologics including but not limited to checkpoint inhibitors (including but not limited to anti-PD1 antibodies such as Keytruda, Opdivo), anti-tumor antigen antibodies (Herceptin), and/or physical methods (surgery, radiation, etc).

In other embodiments, the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

### Administration of GPC3 CAR T Cells

Ortho GPC3 CAR T cells and ortho IL2 can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow infusion), intraarterial, intraperitoneal, intrathecal or subcutaneous administration. In the typical practice, the CAR T cells are infused to the subject in a physiologically acceptable medium, normally intravascularly, although they may also be introduced into any other convenient site, where the cells may find an appropriate site for growth.

Therapeutically effective amounts of ortho GPC3 CAR T cells for use in the method of treating a subject suffering from a GPC+ neoplasm are from about 1×10⁴ to 5×10⁶ ortho GPC3 CAR T cells per kg of subject bodyweight per course of therapy. In some embodiments, the dose ortho GPC3 CAR T cells administered to the subject in need of treatment in a course of therapy is approximately 1×10⁵, alternatively 2×10⁵, alternatively 3×10⁵, alternatively 4×10⁵, alternatively 5×10⁵, alternatively 6×10⁵, alternatively 7×10⁵, alternatively 8×10⁵, alternatively 9×10⁵, alternatively 1×10⁶, alternatively 2×10⁶, alternatively 3×10⁶, alternatively 4×10⁶, alternatively 5×10⁶ ortho GPC3 CAR cells/kg of bodyweight.

A course of ortho GPC3 CAR T cell therapy may be a single dose or in multiple doses over a period of time. Typically, the Ortho GPC3 CAR T cells are administered in a single dose per course of therapy and the ortho IL2 administered periodically in combination with the ortho GPC3 CAR T cells. However, the Ortho GPC3 CAR T cells may be administered in two or more split doses administered over a period of 1, 2, 3, 4, 5, 6, 7, 8, 9, 10, 11, 12, 13, 14, 21, 28, 30, 60, 90, 120 or 180 days. The quantity of ortho GPC3 CAR T cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

### Administration of Ortho IL2:

In embodiments of the therapeutic methods of the present disclosure involve the administration of a pharmaceutical formulation comprising an therapeutically effective amount of ortho IL2 (and/or nucleic acids encoding the ortho IL2) to a subject in need of treatment. Administration to the subject may be achieved by intravenous, as a bolus or by continuous infusion over a period of time. Alternative routes of administration include intramuscular, intraperitoneal, intra-cerobrospinal, subcutaneous, intra-articular, intrasynovial, intrathecal, oral, topical, or inhalation routes. The ortho IL2s also are suitably administered by intratumoral, peritumoral, intralesional, intranodal or perilesional routes or to the lymph, to exert local as well as systemic therapeutic effects.

When the ortho IL2 is STK-009, a therapeutically effective amount of STK-009 for a human subject in combination with an ortho GPC3 CAR T cell is from about 0.5 mg to about 20 mg, alternatively from about 1 mg to about 15 mg, alternatively from about 1.5 mg to about 12 mg administered subcutaneously weekly.

In some embodiments, subject ortho IL2s (and/or nucleic acids encoding the ortho IL2) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the ortho IL2 is to be administered to the subject in need of treatment or prophyaxis.

The ortho IL2s (or nucleic acids encoding same) of the present disclosure may be administered to a subject in a pharmaceutically acceptable dosage form. The preferred formulation depends on the intended mode of administration and therapeutic application. Typically, protein therapeutics are formulated for parenteral administration and the methods of the present disclosure involve the parental administration of an ortho IL2. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for parenteral administration

In some embodiments of the method of the present disclosure, the ortho IL2 may administered to a subject in need of treatment in a formulation to provide extended release of the ortho IL2 agent. Examples of extended release formulations of the injectable compositions can be brought about by including in the composition an agent which delays absorption, for example, aluminum monostearate and gelatin. In one embodiment, the subject ortho IL2s or nucleic acids are prepared with carriers that will protect the mutant IL-2 polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. In one embodiment, the ortho IL2 formulation is provided in accordance with the teaching of Fernandes and Taforo, U.S. Pat. No. 4,604,377 issued Aug. 5, 1986 and Yasui, et al., U.S. Pat. No. 4,645,830.

Alternative to the administration to a subject of a ortho IL2 protein pharmaceutical formulation comprising an ortho IL2, the ortho IL2 may be provided to a subject by the administration of pharmaceutically acceptable formulation of a recombinant vector comprising a nucleic acid sequence encoding the ortho IL2 to the subject to achieve continuous exposure of the subject to the selective ortho IL2. The administration of a recombinant vector encoding the ortho IL2 provides for extended delivery of the ortho IL2 to the subject and prolonged activation of the corresponding cells engineered to express the cognate orthogonal receptor associated with such ortho IL2. In some embodiments of the method of the present disclosure, nucleic acids encoding the ortho IL2 is administered to the subject by transfection or infection using methods known in the art, including but not limited to the methods described in McCaffrey et al. (Nature 418:6893, 2002), Xia et al. (Nature Biotechnol. 20:1006-1010, 2002), or Putnam (Am. J. Health Syst. Pharm. 53:151-160, 1996, erratum at Am. J. Health Syst. Pharm. 53:325, 1996.

In some embodiments, the, ortho IL2 may be administered to a subject in the form of nucleic acid expression construct in viral vector encoding the ortho IL2. The terms “viral vector” and “virus” are used interchangeably herein to refer to any of the obligate intracellular parasites having no protein-synthesizing or energy-generating mechanism. The viral genome may be RNA or DNA contained with a coated structure of protein of a lipid membrane. The terms virus(es) and viral vector(s) are used interchangeably herein. The viruses useful in the practice of the present invention include recombinantly modified enveloped or nonenveloped DNA and RNA viruses, preferably selected from baculoviridiae, parvoviridiae, picomoviridiae, herpesviridiae, poxviridae, or adenoviridiae. The viruses are modified by recombinant DNA techniques to include expression of exogenous transgenes (e.g. a nucleic acid sequence encoding the ortho IL2) and may be engineered to be replication deficient, conditionally replicating or replication competent. The term “replication competent viral vectors” refers to a viral vector that is capable of infection, DNA replication, packaging and lysis of an infected cell. The term “conditionally replicating viral vectors” is used herein to refer to replication competent vectors that are designed to achieve selective expression in particular cell types. Such conditional replication may be achieved by operably linking tissue specific, tumor specific or cell type specific or other selectively induced regulatory control sequences to early genes (e.g., the E1 gene of adenoviral vectors). Infection of the subject with the recombinant virus or non-viral vector can provide for long term expression of the ortho IL2 in the subject and provide continuous selective maintenance of the ortho GPC3 CAR T cells that express the CD122 orthogonal receptor. In one embodiment, the nucleic acid sequence in the viral vector system encoding the IL2 receptor is under control of a regulatable promoter, inducible promoter, tissue specific or tumor specific promoter, or temporally regulated promoter.

### With Lymphodepletion

Lymphodepletion regimens are commonly employed in combination with CAR T cell therapy and agents and dose ranges for the administration of lymphodepleting agents are well known in the art. In one embodiment of the practice of the foregoing method, the subject is treated with a lymphodepletion regimen comprising cyclophosphamide in combination with fludarabine. In some embodiments the lymphodepletion regimen involves the administration cyclophosphamide in combination with fludarabine for a period of 1, 2, 3, 4, or 5 days prior to the administration of the GPC3 CAR T cells. In some embodiments the dose of cyclophosphamide used in the lymphodepletion regimen is from about 100, 200, 300, 400, 500, 600 mg/m²/day over the course of 1, 2, 3, 4, or 5 days prior to the administration of the GPC CAR T cells. In some embodiments the dose of fludarabine used in the lymphodepletion regimen is from about 10, 20, 30, 40, 50, 60 mg/m²/day over the course of 1, 2, 3, 4, or 5 days prior to the administration of the GPC CAR T cells. In one embodiment, the dose of cyclophosphamide at about 500 mg/m²/day to about 600 mg/m²/day and a dose of fludarabine at about 30 mg/m²/day for a period of three days prior to administration of the GPC3 CAR T cells. In one embodiment, the dose of cyclophosphamide at about 300 mg/m²/day to about 600 mg/m²/day and a dose of fludarabine at about 30 mg/m²/day for a period of three days prior to administration of the GPC3 CAR T cells.

### Without Lymphodepletion

As previously noted, the GPC3 CAR T cells are able to be expanded in vivo in a mammalian subject. Consequently, this enables the administration of a significant lower dose of GPC3 CAR T cells to be administered to the patient and thereby reducing or eliminating the need for lymphodepletion prior to administration of the GPC3 CAR T cells. Lymphodepletion significantly compromises the subject by leaving them vulnerable to environmental factors and avoiding lymphodepleting regimens in conjunction with cell therapy would be of significant benefit to the patient

In one embodiment, the present disclosure provides a method of treating a subject suffering from a neoplastic disease, disorder or condition the neoplasm characterized by the expression of GPC3 amenable to treatment with GPC3 CAR-T cell therapy by the administration of a orthogonal ligand expressing CAR-Ts in the absence of lymphodepletion.

Sequence Listing

| SEQ | | |
|---|---|---|
| ID | | |
| NO. | Description | Amino Acid or DNA Sequence |
| 1 | Human GPC3 precursor | MAGTVRTACLVVAMLLSLDFPGQAQPPPPPPDAT |
| | (580 amino acids, signal peptide | CHQVRSFFQRLQPGLKWVPETPVPGSDLQVCLPK |
| | underlined; | GPTCCSRKMEEKYQLTARLNMEQLLQSASMELKF |
| | UniProt Ref: P51654 | LIIQNAAVFQEAFEIVVRHAKNYTNAMFKNNYPS |
| | | LTPQAFEFVGEFFTDVSLYILGSDINVDDMVNEL |
| | | FDSLFPVIYTQLMNPGLPDSALDINECLRGARRD |
| | | LKVFGNFPKLIMTQVSKSLQVTRIFLQALNLGIE |
| | | VINTTDHLKFSKDCGRMLTRMWYCSYCQGLMMVK |
| | | PCGGYCNVVMQGCMAGVVEIDKYWREYILSLEEL |
| | | VNGMYRIYDMENVLLGLFSTIHDSIQYVQKNAGK |
| | | LTTTIGKLCAHSQQRQYRSAYYPEDLFIDKKVLK |
| | | VAHVEHEETISSRRRELIQKLKSFISFYSALPGY |
| | | ICSHSPVAENDTLCWNGQELVERYSQKAARNGMK |
| | | NQFNLHELKMKGPEPVVSQIIDKLKHINQLLRTM |
| | | SMPKGRVLDKNLDEEGFESGDCGDDEDECIGGSG |
| | | DGMIKVKNQLRFLAELAYDLDVDDAPGNSQQATP |
| | | KDNEISTFHNLGNVHSPLKLLTSMAISVVCFFFL |
| | | VH |
| 2 | Human Glypican-3 beta subunit | SAYYPEDLFIDKKVLKVAHVEHEETLSSRRRELI |
| | UniProt Ref: P51654, AAs 359- | QKLKSFISFYSALPGYICSHSPVAENDTLCWNGQ |
| | SS4 numbered in accordance | ELVERYSQKAARNGMKNQFNLHELKMKGPEPVVS |
| | with hGPC3 precursor, SEQ ID | QIIDKLKHINQLLRTMSMPKGRVLDKNLDEEGFE |
| | NO: 1 | SGDCGDDEDECIGGSGDGMIKVKNQLRFLAELAY |
| | | DLDVDDAPGNSQQATPKDNEISTFHN |
| 3 | Wild Type Human CD122 | MAAPALSWRLPLLILLLPLATSWASAAVNGTSQF |
| | precursor (including signal | TCFYNSRANISCVWSQDGALQDTSCQVHAWPDRR |
| | peptide underlined) | RWNQTCELLPVSQASWACNLILGAPDSQKLTTVD |
| | UniProt Ref: P14784 | IVTLRVLCREGVRWRVMAIQDFKPFENLRLMAPI |
| | | SLQVVHVETHRCNISWEISQASHYFERHLEFEAR |
| | | TLSPGHTWEEAPLLTLKQKQEWICLETLTPDTQY |
| | | EFQVRVKPLQGEFTTWSPWSQPLAFRTKPAALGK |
| | | DTIPWLGHLLVGLSGAFGFIILVYLLINCRNTGP |
| | | WLKKVLKCNTPDPSKFFSQLSSEHGGDVQKWLSS |
| | | PFPSSSFSPGGLAPEISPLEVLERDKVTQLLLQQ |
| | | DKVPEPASLSSNHSLTSCFTNQGYFFFHLPDALE |
| | | IEACQVYFTYDPYSEEDPDEGVAGAPTGSSPQPL |
| | | QPLSGEDDAYCTFPSRDDLLLFSPSLLGGPSPPS |
| | | TAPGGSGAGEERMPPSLQERVPRDWDPQPLGPPT |
| | | PGVPDLVDFQPPPELVLREAGEEVPDAGPREGVS |
| | | FPWSRPPGQGEFRALNARLPLNTDAYLSLQELQG |
| | | QDPTHLV |
| 4 | Mature wild type hCD122 | AVNGTSQFTCFYNSRANISCVWSQDGALQDTSCQ |
| | (without signal peptide) | VHAWPDRRRWNQTCELLPVSQASWACNLILGAPD |
| | UniProt Ref: P14784 | SQKLTTVDIVTLRVLCREGVRWRVMAIQDFKPFE |
| | | NLRLMAPISLQVVHVETHRCNISWEISQASHYFE |
| | | RHLEFEARTLSPGHTWEEAPLLTLKQKQEWICLE |
| | | TLTPDTQYEFQVRVKPLQGEFTTWSPWSQPLAFR |
| | | TKPAALGKDTIPWLGHLLVGLSGAFGFIILVYLL |
| | | INCRNTGPWLKKVLKCNTPDPSKFFSQLSSEHGG |
| | | DVQKWLSSPFPSSSFSPGGLAPEISPLEVLERDK |
| | | VTQLLLQQDKVPEPASLSSNHSLTSCFTNQGYFF |
| | | FHLPDALEIEACQVYFTYDPYSEEDPDEGVAGAP |
| | | TGSSPQPLQPLSGEDDAYCTFPSRDDLLLFSPSL |
| | | LGGPSPPSTAPGGSGAGEERMPPSLQERVPRDWD |
| | | PQPLGPPTPGVPDLVDFQPPPELVLREAGEEVPD |
| | | AGPREGVSFPWSRPPGQGEFRALNARLPLNTDAY |
| | | LSLQELQGQDPTHLV |
| 5 | hoCD122 precursor | MAAPALSWRLPLLILLLPLATSWASAAVNGTSQF |
| | with signal peptide (underlined) | TCFYNSRANISCVWSQDGALQDTSCQVHAWPDRR |
| | containing amino acid | RWNQTCELLPVSQASWACNLILGAPDSQKLTTVD |
| | substitutions H133D and Y134F | IVTLRVLCREGVRWRVMAIQDFKPFENLRLMAPI |
| | (bold, numbered in accordance | SLQVVHVETHRCNISWEISQASDFFERHLEFEAR |
| | with mature wt CD122 protein, | TLSPGHTWEEAPLLTLKQKQEWICLETLTPDTQY |
| | SEQ ID NO: 4) | EFQVRVKPLQGEFTTWSPWSQPLAFRTKPAALGK |
| | | DTIPWLGHLLVGLSGAFGFIILVYLLINCRNTGP |
| | | WLKKVLKCNTPDPSKFFSQLSSEHGGDVQKWLSS |
| | | PFPSSSFSPGGLAPEISPLEVLERDKVTQLLLQQ |
| | | DKVPEPASLSSNHSLTSCFTNQGYFFFHLPDALE |
| | | IEACQVYFTYDPYSEEDPDEGVAGAPTGSSPQPL |
| | | QPLSGEDDAYCTFPSRDDLLLESPSLLGGPSPPS |
| | | TAPGGSGAGEERMPPSLQERVPRDWDPQPLGPPT |
| | | PGVPDLVDFQPPPELVLREAGEEVPDAGPREGVS |
| | | FPWSRPPGQGEFRALNARLPLNTDAYLSLQELQG |
| | | QDPTHLV |
| 6 | Mature hoCD122 | AVNGTSQFTCFYNSRANISCVWSQDGALQDTSCQ |
| | without signal peptide | VHAWPDRRRWNQTCELLPVSQASWACNLILGAPD |
| | containing amino acid | SQKLTTVDIVTLRVLCREGVRWRVMAIQDFKPFE |
| | substitutions H133D and Y134F | NLRLMAPISLQVVHVETHRCNISWEISQASDFFE |
| | (bold, numbered in accordance | RHLEFEARTLSPGHTWEEAPLLTLKQKQEWICLE |
| | with mature wt CD122 protein | TLTPDTQYEFQVRVKPLQGEFTTWSPWSQPLAFR |
| | SEQ ID NO: 4) | TKPAALGKDTIPWLGHLLVGLSGAFGFIILVYLL |
| | | INCRNTGPWLKKVLKCNTPDPSKFFSQLSSEHGG |
| | | DVQKWLSSPFPSSSESPGGLAPEISPLEVLERDK |
| | | VTQLLLQQDKVPEPASLSSNHSLTSCFTNQGYFF |
| | | FHLPDALEIEACQVYFTYDPYSEEDPDEGVAGAP |
| | | TGSSPQPLQPLSGEDDAYCTFPSRDDLLLESPSL |
| | | LGGPSPPSTAPGGSGAGEERMPPSLQERVPRDWD |
| | | PQPLGPPTPGVPDLVDFQPPPELVLREAGEEVPD |
| | | AGPREGVSFPWSRPPGQGEFRALNARLPLNTDAY |
| | | LSLQELQGQDPTHLV |
| 7 | Wild Type Human IL2 Precursor | MYRMQLLSCIALSLALVTNSAPTSSSTKKTQLQL |
| | (signal peptide underlined) | EHLLLDLQMILNGINNYKNPKLTRMLTFKFYMPK |
| | UniProt P60568) | KATELKHLQCLEEELKPLEEVLNLAQSKNFHLRP |
| | | RDLISNINVIVLELKGSETTFMCEYADETATIVE |
| | | FLNRWITFCQSIISTLT |
| 8 | Mature Wild Type human IL2 | APTSSSTKKTQLQLEHLLLDLQMILNGINNYKNP |
| | UniProt P60568 | KLTRMLTFKFYMPKKATELKHLQCLEEELKPLEE |
| | | VLNLAQSKNFHLRPRDLISNINVIVLELKGSETT |
| | | FMCEYADETATIVEFLNRWITFCQSIISTLT |
| 9 | hIL2 mutein having elation of | PTSSSTKKTQLQLSQLLVLLKAILNGINNYKNPK |
| | Ala1 and AA subsitutions | LTRMLTFKFYMPKKATELKHLQCLEEELKPLEEV |
| | E15S/H16Q/L19V/D20L/Q22K/ | LNLAQSKNFHLRPRDLISNINVIVLELKGSETTE |
| | M23A (STK-007) | MCEYADETATIVEFLNRWITFCQSIISTLT |
| 10 | GC33 scFv with G4Sx3 linker | DVVMTQSPLSLPVTPGEPASISCRSSQSLVHSNA |
| | (underlined) | NTYLHWYLQKPGQSPQLLIYKVSNRFSGVPDRFS |
| | | GSGSGTDFTLKISRVEAEDVGVYYCSQNTHVPPT |
| | | FGQGTKLEIKRGGGGSGGGGSGGGGSQVQLVQSG |
| | | AEVKKPGASVKVSCKASGYTFTDYEMHWVRQAPG |
| | | QGLEWMGALDPKTGDTAYSQKFKGRVTLTADEST |
| | | STAYMELSSLRSEDTAVYYCTRFYSYTYWGQGTL |
| | | VTVSS |
| 11 | GC33 scFv with Whitlow linker | DVVMTQSPLSLPVTPGEPASISCRSSQSLVHSNA |
| | (underlined) | NTYLHWYLQKPGQSPQLLIYKVSNRFSGVPDRFS |
| | | GSGSGTDFTLKISRVEAEDVGVYYCSQNTHVPPT |
| | | FGQGTKLEIKRGSTSGSGKPGSGEGSTKGQVQLV |
| | | QSGAEVKKPGASVKVSCKASGYTFTDYEMHWVRQ |
| | | APGQGLEWMGALDPKTGDTAYSQKFKGRVTLTAD |
| | | ESTSTAYMELSSLRSEDTAVYYCTRFYSYTYWGQ |
| | | GTLVTVSS |
| 12 | GC33 Heavy Chain CDR1 | DYEMH |
| 13 | GC33 Heavy Chain CDR2 | ALDPKTGDTAYSQKFKG |
| 14 | GC33 Heavy Chain CDR3 | FYSYTY |
| 15 | GC33 Light Chain CDR1 | RSSQSLVHSNANTYLH |
| 16 | GC33 Light Chain CDR2 | KVSNRFS |
| 17 | GC33 Light Chain CDR3 | SQNTHVPPT |
| 18 | Whitlow Linker | GSTSGSGKPGSGEGSTKG |
| 19 | hCD8A precursor including | MALPVTALLLPLALLLHAARPSQFRVSPLDRTWN |
| | signal peptide (underlined), | LGETVELKCQVLLSNPTSGCSWLFQPRGAAASPT |
| | UniProt Ref: P01732 | FLLYLSQNKPKAAEGLDTQRFSGKRLGDTFVLTL |
| | | SDFRRENEGYYFCSALSNSIMYFSHFVPVFLPAK |
| | | PTTTPAPRPPTPAPTIASQPLSLRPEACRPAAGG |
| | | AVHTRGLDFACDIYIWAPLAGTCGVLLLSLVITL |
| | | YCNHRNRRRVCKCPRPVVKSGDKPSLSARYV |
| 20 | Human CD8a signal peptide | MALPVTALLLPLALLLHAARP |
| | (AAs 1-21 of hCD8a precursor | |
| | numbered in accordance with | |
| | hCD8A precursor including | |
| | signal peptide, UniProt Ref: | |
| | P01732) | |
| 21 | Human CD8a hinge | ALSNSIMYFSHFVPVFLPAKPTTTPAPRPPTPAP |
| | 117 to 178 | TIASQPLSLRPEACRPAAGGAVHTRGLD |
| 22 | Human CD8a hinge fragment | TTTPAPRPPTPAPTIASQPLSLRPEACRPAAGGA |
| | | VHTRGLD |
| 23 | Human CD8a hinge region with | ALSNSIMYFSHFVPVFLPAKPTTTPAPRPPTPAP |
| | modification of unpaired CYS to | TIASQPLSLRPEASRPAAGGAVHTRGLD |
| | SER substitution at | |
| | 117 to 178; Schonfeld, et al | |
| | European patent application | |
| | 3115373A1 | |
| 24 | Human CD8a transmembrane | FACDIYIWAPLAGTCGVLLLSLVITLYC |
| | domain 1 | |
| 25 | Human CD8a transmembrane | FACDIYIWAPLAGTCGVLLLSLVIT |
| | domain 2 | |
| 26 | Partial human CD8a hinge | TTTPAPRPPTPAPTIASQPLSLRPEACRPAAGGA |
| | domain, CD8a transmembrane | VHTRGLDFACDIYIWAPLAGTCGVLLLSLVITLY |
| | domain (AAs 138-206 of hCD8a | C |
| | precursor numbered in | |
| | accordance with hCD8A | |
| | precursor including signal | |
| | peptide, UniProt Ref: P01732) | |
| 27 | Human CD8a hinge and | TTTPAPRPPTPAPTIASQPLSLRPEACRPAAGGA |
| | transmembrane domain | VHTRGLDFACDIYIWAPLAGTCGVLLLSLVIT |
| | (AAs 138-203 of hCD8a | |
| | precursor numbered in | |
| | accordance with hCD8A | |
| | precursor including signal | |
| | peptide, UniProt Ref: P01732) | |
| 28 | Human CD28 ECD fragment, | IEVMYPPPYLDNEKSNGTIIHVKGKHLCPSPLFP |
| | transmembrane and Co- | GPSKPFWVLVVVGGVLACYSLLVTVAFIIFWVRS |
| | stimulatory domain | KRSRLLHSDYMNMTPRRPGPTRKHYQPYAPPRDF |
| | (UniProt P10747, AAs114-220 | AAYRS |
| | numbered in accordance with | |
| | precursor including signal | |
| | peptide) | |
| 29 | Human CD28 ECD fragment | IEVMYPPPYLDNEKSNGTIIHVKGKHLCPSPLFP |
| | | GPSKPF |
| 30 | Human CD28 transmembrane | WVLVVVGGVLACYSLLVTVAFIIFW |
| | domain | |
| 31 | Human CD28 co-stimulatory | VRSKRSRLLHSDYMNMTPRRPGPTRKHYQPYAPP |
| | domain | RDFAAYRS |
| 32 | Human 4-1BB | KRGRKKLLYIFKQPFMRPVQTTQEEDGCSCRFPE |
| | (CD137,TNFRSF9) co- | EEEGGCEL |
| | stimulatory domain | |
| | (UniProt Q07011, AAs 129-225 | |
| | numbered in accordance with | |
| | precursor including signal | |
| | peptide) | |
| 33 | Human CD3 zeta domain | RVKFSRSADAPAYQQGQNQLYNELNLGRREEYDV |
| | (UniProt P20963, AAs 52-164 | LDKRRGRDPEMGGKPRRKNPQEGLYNELQKDKMA |
| | numbered in accordance with | EAYSEIGMKGERRRGKGHDGLYQGLSTATKDTYD |
| | CD3z precursor including signal | ALHMQALPPR |
| | peptide) | |
| 34 | hCD8a signal peptide (italics); | MALPVTALLLPLALLLHAARPDVVMTQSPLSLPV |
| | Whitlow Linker underlined); | TPGEPASISCRSSQSLVHSNANTYLHWYLQKPGQ |
| | hCD28 transmembrane and co- | SPQLLIYKVSNRFSGVPDRFSGSGSGTDFTLKIS |
| | stimulatory domain; | RVEAEDVGVYYCSQNTHVPPTFGQGTKLEIKRGS |
| | hCD3zeta domain | TSGSGKPGSGEGSTKGQVQLVQSGAEVKKPGASV |
| | | KVSCKASGYTFTDYEMHWVRQAPGQGLEWMGALD |
| | | PKTGDTAYSQKFKGRVTLTADESTSTAYMELSSL |
| | | RSEDTAVYYCTRFYSYTYWGQGTLVTVSSIEVMY |
| | | PPPYLDNEKSNGTIIHVKGKHLCPSPLFPGPSKP |
| | | FWVLVVVGGVLACYSLLVTVAFIIFWVRSKRSRL |
| | | LHSDYMNMTPRRPGPTRKHYQPYAPPRDFAAYRS |
| | | RVKFSRSADAPAYQQGQNQLYNELNLGRREEYDV |
| | | LDKRRGRDPEMGGKPRRKNPQEGLYNELQKDKMA |
| | | EAYSEIGMKGERRRGKGHDGLYQGLSTATKDTYD |
| | | ALHMQALPPR |
| 35 | hCD8a signal peptide (italics); | MALPVTALLLPLALLLHAARPDVVMTQSPLSLPV |
| | G4Sx3 Linker (underlined); | TPGEPASISCRSSQSLVHSNANTYLHWYLQKPGQ |
| | hCD28 transmembrane and co- | SPQLLIYKVSNRFSGVPDRFSGSGSGTDFTLKIS |
| | stimulatory domain; | RVEAEDVGVYYCSQNTHVPPTFGQGTKLEIKRGG |
| | hCD3zeta domain | GGSGGGGSGGGGSQVQLVQSGAEVKKPGASVKVS |
| | | CKASGYTFTDYEMHWVRQAPGQGLEWMGALDPKT |
| | | GDTAYSQKFKGRVTLTADESTSTAYMELSSLRSE |
| | | DTAVYYCTRFYSYTYWGQGTLVTVSSIEVMYPPP |
| | | YLDNEKSNGTIIHVKGKHLCPSPLFPGPSKPFWV |
| | | LVVVGGVLACYSLLVTVAFIIFWVRSKRSRLLHS |
| | | DYMNMTPRRPGPTRKHYQPYAPPRDFAAYRSRVK |
| | | FSRSADAPAYQQGQNQLYNELNLGRREEYDVLDK |
| | | RRGRDPEMGGKPRRKNPQEGLYNELQKDKMAEAY |
| | | SEIGMKGERRRGKGHDGLYQGLSTATKDTYDALH |
| | | MQALPPR |
| 36 | · CD8a signal peptide | MALPVTALLLPLALLLHAARPDVVMTQSPLSLPV |
| | (italics), GPC3 scFv | TPGEPASISCRSSQSLVHSNANTYLHWYLQKPGQ |
| | whitlow linker | SPQLLIYKVSNRFSGVPDRFSGSGSGTDFTLKIS |
| | underlined, hCD8a hinge | RVEAEDVGVYYCSQNTHVPPTFGQGTKLEIKRGS |
| | and transmembrane h4- | TSGSGKPGSGEGSTKGQVQLVQSGAEVKKPGASV |
| | 1BB co-stimulatory | KVSCKASGYTFTDYEMHWVRQAPGQGLEWMGALD |
| | domain, hCD3zeta | PKTGDTAYSQKFKGRVTLTADESTSTAYMELSSL |
| | domain | RSEDTAVYYCTRFYSYTYWGQGTLVTVSSTTTPA |
| | | PRPPTPAPTIASQPLSLRPEACRPAAGGAVHTRG |
| | | LDFACDIYIWAPLAGTCGVLLLSLVITLYCKRGR |
| | | KKLLYIFKQPFMRPVQTTQEEDGCSCRFPEEEEG |
| | | GCELRVKFSRSADAPAYQQGQNQLYNELNLGRRE |
| | | EYDVLDKRRGRDPEMGGKPRRKNPQEGLYNELQK |
| | | DKMAEAYSEIGMKGERRRGKGHDGLYQGLSTATK |
| | | DTYDALHMQALPPR |
| 37 | DR625 GPC3 CAR | MALPVTALLLPLALLLHAARPDVVMTQSPLSLPV |
| | | TPGEPASISCRSSQSLVHSNANTYLHWYLQKPGQ |
| | | SPQLLIYKVSNRFSGVPDRFSGSGSGTDFTLKIS |
| | | RVEAEDVGVYYCSQNTHVPPTFGQGTKLEIKRGS |
| | | TSGSGKPGSGEGSTKGQVQLVQSGAEVKKPGASV |
| | | KVSCKASGYTFTDYEMHWVRQAPGQGLEWMGALD |
| | | PKTGDTAYSQKFKGRVTLTADESTSTAYMELSSL |
| | | RSEDTAVYYCTRFYSYTYWGQGTLVTVSSAAAIE |
| | | VMYPPPYLDNEKSNGTIIHVKGKHLCPSPLFPGP |
| | | SKPFWVLVVVGGVLACYSLLVTVAFIIFWVRSKR |
| | | SRLLHSDYMNMTPRRPGPTRKHYQPYAPPRDFAA |
| | | YRSRVKFSRSADAPAYQQGQNQLYNELNLGRREE |
| | | YDVLDKRRGRDPEMGGKPRRKNPQEGLYNELQKD |
| | | KMAEAYSEIGMKGERRRGKGHDGLYQGLSTATKD |
| | | TYDALHMQALPPRGSGEGRGSLLTCGDVEENPGP |
| | | MAAPALSWRLPLLILLLPLATSWASAAVNGTSQF |
| | | TCFYNSRANISCVWSQDGALQDTSCQVHAWPDRR |
| | | RWNQTCELLPVSQASWACNLILGAPDSQKLTTVD |
| | | IVTLRVLCREGVRWRVMAIQDFKPFENLRLMAPI |
| | | SLQVVHVETHRCNISWEISQASDFFERHLEFEAR |
| | | TLSPGHTWEEAPLLTLKQKQEWICLETLTPDTQY |
| | | EFQVRVKPLQGEFTTWSPWSQPLAFRTKPAALGK |
| | | DTIPWLGHLLVGLSGAFGFIILVYLLINCRNTGP |
| | | WLKKVLKCNTPDPSKFFSQLSSEHGGDVQKWLSS |
| | | PFPSSSESPGGLAPEISPLEVLERDKVTQLLLQQ |
| | | DKVPEPASLSSNHSLTSCFTNQGYFFFHLPDALE |
| | | IEACQVYFTYDPYSEEDPDEGVAGAPTGSSPQPL |
| | | QPLSGEDDAYCTFPSRDDLLLFSPSLLGGPSPPS |
| | | TAPGGSGAGEERMPPSLQERVPRDWDPQPLGPPT |
| | | PGVPDLVDFQPPPELVLREAGEEVPDAGPREGVS |
| | | FPWSRPPGQGEFRALNARLPLNTDAYLSLQELQG |
| | | QDPTHLV |
| 38 | DR626 GPC3 CAR | MAAPALSWRLPLLILLLPLATSWASAAVNGTSQF |
| | | TCFYNSRANISCVWSQDGALQDTSCQVHAWPDRR |
| | | RWNQTCELLPVSQASWACNLILGAPDSQKLTTVD |
| | | IVTLRVLCREGVRWRVMAIQDFKPFENLRLMAPI |
| | | SLQVVHVETHRCNISWEISQASDFFERHLEFEAR |
| | | TLSPGHTWEEAPLLTLKQKQEWICLETLTPDTQY |
| | | EFQVRVKPLQGEFTTWSPWSQPLAFRTKPAALGK |
| | | DTIPWLGHLLVGLSGAFGFIILVYLLINCRNTGP |
| | | WLKKVLKCNTPDPSKFFSQLSSEHGGDVQKWLSS |
| | | PFPSSSFSPGGLAPEISPLEVLERDKVTQLLLQQ |
| | | DKVPEPASLSSNHSLTSCFTNQGYFFFHLPDALE |
| | | IEACQVYFTYDPYSEEDPDEGVAGAPTGSSPQPL |
| | | QPLSGEDDAYCTFPSRDDLLLFSPSLLGGPSPPS |
| | | TAPGGSGAGEERMPPSLQERVPRDWDPQPLGPPT |
| | | PGVPDLVDFQPPPELVLREAGEEVPDAGPREGVS |
| | | FPWSRPPGQGEFRALNARLPLNTDAYLSLQELQG |
| | | QDPTHLVGSGEGRGSLLTCGDVEENPGPMALPVT |
| | | ALLLPLALLLHAARPDVVMTQSPLSLPVTPGEPA |
| | | SISCRSSQSLVHSNANTYLHWYLQKPGQSPQLLI |
| | | YKVSNRFSGVPDRFSGSGSGTDFTLKISRVEAED |
| | | VGVYYCSQNTHVPPTFGQGTKLEIKRGSTSGSGK |
| | | PGSGEGSTKGQVQLVQSGAEVKKPGASVKVSCKA |
| | | SGYTFTDYEMHWVRQAPGQGLEWMGALDPKTGDT |
| | | AYSQKFKGRVTLTADESTSTAYMELSSLRSEDTA |
| | | VYYCTRFYSYTYWGQGTLVTVSSAAAIEVMYPPP |
| | | YLDNEKSNGTIIHVKGKHLCPSPLFPGPSKPFWV |
| | | LVVVGGVLACYSLLVTVAFIIFWVRSKRSRLLHS |
| | | DYMNMTPRRPGPTRKHYQPYAPPRDFAAYRSRVK |
| | | FSRSADAPAYQQGQNQLYNELNLGRREEYDVLDK |
| | | RRGRDPEMGGKPRRKNPQEGLYNELQKDKMAEAY |
| | | SEIGMKGERRRGKGHDGLYQGLSTATKDTYDALH |
| | | MQALPPR |
| 39 | DR628 GPC3 CAR | MALPVTALLLPLALLLHAARPDVVMTQSPLSLPV |
| | | TPGEPASISCRSSQSLVHSNANTYLHWYLQKPGQ |
| | | SPQLLIYKVSNRFSGVPDRFSGSGSGTDFTLKIS |
| | | RVEAEDVGVYYCSQNTHVPPTFGQGTKLEIKRGS |
| | | TSGSGKPGSGEGSTKGQVQLVQSGAEVKKPGASV |
| | | KVSCKASGYTFTDYEMHWVRQAPGQGLEWMGALD |
| | | PKTGDTAYSQKFKGRVTLTADESTSTAYMELSSL |
| | | RSEDTAVYYCTRFYSYTYWGQGTLVTVSSTTTPA |
| | | PRPPTPAPTIASQPLSLRPEACRPAAGGAVHTRG |
| | | LDFACDIYIWAPLAGTCGVLLLSLVITKRGRKKL |
| | | LYIFKQPFMRPVQTTQEEDGCSCRFPEEEEGGCE |
| | | LRVKFSRSADAPAYQQGQNQLYNELNLGRREEYD |
| | | VLDKRRGRDPEMGGKPQRRKNPQEGLYNELQKDK |
| | | MAEAYSEIGMKGERRRGKGHDGLYQGLSTATKDT |
| | | YDALHMQALPPR |
| 40 | DR1581 CAR | MALPVTALLLPLALLLHAARPDVVMTQSPLSLPV |
| | (DR625 with CD28 replaced | TPGEPASISCRSSQSLVHSNANTYLHWYLQKPGQ |
| | with CD8a TM 41bb) | SPQLLIYKVSNRFSGVPDRFSGSGSGTDFTLKIS |
| | | RVEAEDVGVYYCSQNTHVPPTFGQGTKLEIKRGS |
| | | TSGSGKPGSGEGSTKGQVQLVQSGAEVKKPGASV |
| | | KVSCKASGYTFTDYEMHWVRQAPGQGLEWMGALD |
| | | PKTGDTAYSQKFKGRVTLTADESTSTAYMELSSL |
| | | RSEDTAVYYCTRFYSYTYWGQGTLVTVSSTTTPA |
| | | PRPPTPAPTIASQPLSLRPEACRPAAGGAVHTRG |
| | | LDFACDIYIWAPLAGTCGVLLLSLVITLYCKRGR |
| | | KKLLYIFKQPFMRPVQTTQEEDGCSCRFPEEEEG |
| | | GCELRVKFSRSADAPAYQQGQNQLYNELNLGRRE |
| | | EYDVLDKRRGRDPEMGGKPRRKNPQEGLYNELQK |
| | | DKMAEAYSEIGMKGERRRGKGHDGLYQGLSTATK |
| | | DTYDALHMQALPPRGSGEGRGSLLTCGDVEENPG |
| | | PMAAPALSWRLPLLILLLPLATSWASAAVNGTSQ |
| | | FTCFYNSRANISCVWSQDGALQDTSCQVHAWPDR |
| | | RRWNQTCELLPVSQASWACNLILGAPDSQKLTTV |
| | | DIVTLRVLCREGVRWRVMAIQDFKPFENLRLMAP |
| | | ISLQVVHVETHRCNISWEISQASDFFERHLEFEA |
| | | RTLSPGHTWEEAPLLTLKQKQEWICLETLTPDTQ |
| | | YEFQVRVKPLQGEFTTWSPWSQPLAFRTKPAALG |
| | | KDTIPWLGHLLVGLSGAFGFIILVYLLINCRNTG |
| | | PWLKKVLKCNTPDPSKFFSQLSSEHGGDVQKWLS |
| | | SPFPSSSFSPGGLAPEISPLEVLERDKVTQLLLQ |
| | | QDKVPEPASLSSNHSLTSCFTNQGYFFFHLPDAL |
| | | EIEACQVYFTYDPYSEEDPDEGVAGAPTGSSPQP |
| | | LQPLSGEDDAYCTFPSRDDLLLFSPSLLGGPSPP |
| | | STAPGGSGAGEERMPPSLQERVPRDWDPQPLGPP |
| | | TPGVPDLVDFQPPPELVLREAGEEVPDAGPREGV |
| | | SFPWSRPPGQGEFRALNARLPLNTDAYLSLQELQ |
| | | GQDPTHLV |
| 41 | DNA Sequence Encoding T2A | GGGAGTGGAGAGGGCCGCGGATCACTTCTCACAT |
| | (ribosome skipping sequence, | GCGGCGACGTAGAGGAAAATCCTGGCCCC |
| | not translated into protein) | |
| 42 | DNA Sequence Encoding | ATGGCTCTGCCTGTGACAGCTCTGCTGCTGCCTC |
| | DR625 | TGGCTCTGCTTCTGCATGCCGCCAGACCTGACGT |
| | | GGTCATGACACAGAGCCCTCTGAGCCTGCCTGTG |
| | | ACACCTGGCGAACCTGCCAGCATCAGCTGTAGAA |
| | | GCAGCCAGAGCCTGGTGCACAGCAACGCCAACAC |
| | | ATACCTGCACTGGTATCTGCAGAAGCCCGGCCAG |
| | | TCTCCTCAGCTGCTGATCTACAAGGTGTCCAACA |
| | | GATTCAGCGGCGTGCCCGACAGATTCTCTGGCTC |
| | | TGGATCTGGCACCGACTTCACCCTGAAGATCTCC |
| | | AGAGTGGAAGCCGAGGACGTGGGCGTGTACTACT |
| | | GCAGCCAGAACACACACGTGCCACCTACATTCGG |
| | | CCAGGGCACCAAGCTGGAAATCAAGAGGGGATCC |
| | | ACAAGCGGCTCTGGCAAGCCTGGAAGCGGAGAGG |
| | | GATCTACCAAAGGCCAGGTGCAGCTGGTTCAGTC |
| | | TGGCGCCGAAGTGAAGAAACCTGGCGCCTCTGTG |
| | | AAGGTGTCCTGCAAGGCAAGCGGCTACACATTCA |
| | | CCGACTACGAGATGCACTGGGTCCGACAGGCTCC |
| | | AGGACAGGGACTTGAATGGATGGGAGCCCTAGAT |
| | | CCTAAGACCGGCGATACAGCCTACAGCCAGAAAT |
| | | TCAAGGGAAGAGTGACCCTGACCGCCGACGAGAG |
| | | CACATCTACCGCCTACATGGAACTGAGCAGCCTG |
| | | AGAAGCGAGGACACCGCCGTGTATTACTGTACCA |
| | | GATTCTACTCCTACACCTACTGGGGCCAGGGAAC |
| | | CCTGGTCACAGTCTCTTCTGCCGCCGCTATCGAA |
| | | GTGATGTACCCTCCTCCTTACCTGGACAACGAGA |
| | | AGTCCAACGGCACCATCATCCACGTGAAGGGCAA |
| | | GCACCTGTGTCCTTCTCCACTGTTCCCCGGACCT |
| | | AGCAAGCCTTTCTGGGTGCTCGTTGTTGTTGGCG |
| | | GCGTGCTGGCCTGTTACTCTCTGCTGGTTACCGT |
| | | GGCCTTCATCATCTTTTGGGTCCGATCCAAGCGG |
| | | AGCCGGCTGCTGCACAGCGACTACATGAACATGA |
| | | CCCCTAGACGGCCCGGACCAACCAGAAAGCACTA |
| | | CCAGCCTTACGCTCCTCCTCGGGATTTCGCCGCC |
| | | TACAGATCCAGAGTGAAGTTCTCCAGAAGCGCCG |
| | | ACGCTCCTGCCTATCAGCAGGGACAGAATCAGCT |
| | | GTACAACGAGCTGAACCTGGGGCGCAGAGAAGAG |
| | | TACGACGTGCTGGATAAGCGGAGAGGCAGAGATC |
| | | CTGAGATGGGCGGCAAGCCCAGACGGAAGAATCC |
| | | TCAAGAGGGCCTGTATAATGAGCTGCAGAAAGAC |
| | | AAGATGGCCGAGGCCTACAGCGAGATCGGAATGA |
| | | AGGGCGAACGCAGAAGAGGCAAGGGCCACGATGG |
| | | ACTGTACCAGGGACTGAGCACCGCCACCAAGGAT |
| | | ACCTATGACGCCCTGCACATGCAGGCTCTGCCAC |
| | | CTAGAGGGTCCGGAGAAGGCAGAGGATCTCTGCT |
| | | GACATGCGGCGACGTGGAAGAGAACCCTGGGCCC |
| | | ATGGCTGCCCCTGCTCTGTCTTGGAGACTGCCTC |
| | | TGCTGATCCTGCTGCTGCCTCTGGCTACATCTTG |
| | | GGCCTCTGCCGCCGTGAATGGCACCAGCCAGTTC |
| | | ACCTGTTTCTACAACAGCCGGGCCAACATCAGCT |
| | | GCGTGTGGTCACAAGATGGCGCCCTGCAGGATAC |
| | | CAGCTGTCAGGTTCACGCCTGGCCTGATCGGAGA |
| | | AGATGGAACCAGACATGCGAGCTGCTGCCCGTGT |
| | | CTCAGGCTTCCTGGGCCTGTAATCTGATCCTGGG |
| | | AGCCCCTGACAGCCAGAAACTGACCACCGTGGAT |
| | | ATCGTGACCCTGCGGGTGCTGTGTAGAGAAGGCG |
| | | TTCGTTGGAGAGTGATGGCCATTCAGGACTTCAA |
| | | GCCCTTCGAGAACCTGCGGCTGATGGCCCCAATT |
| | | AGCCTGCAGGTTGTGCACGTGGAAACCCACAGAT |
| | | GCAATATCAGCTGGGAGATCAGCCAGGCCAGCGA |
| | | CTTCTTCGAGCGGCACCTGGAATTTGAGGCCCGG |
| | | ACACTGTCTCCAGGCCACACCTGGGAAGAAGCCC |
| | | CTCTGCTCACCCTGAAGCAGAAACAAGAGTGGAT |
| | | CTGCCTGGAAACCCTGACACCTGACACACAGTAC |
| | | GAGTTCCAAGTGCGCGTGAAGCCTCTCCAGGGCG |
| | | AGTTCACGACGTGGTCCCCTTGGAGTCAGCCCCT |
| | | GGCCTTTAGAACAAAGCCTGCCGCTCTGGGCAAA |
| | | GACACAATCCCTTGGCTGGGCCATCTGCTCGTTG |
| | | GACTGTCTGGCGCCTTCGGCTTCATCATCCTGGT |
| | | GTACCTGCTGATCAACTGCCGGAACACCGGACCT |
| | | TGGCTGAAGAAAGTGCTGAAGTGCAACACCCCTG |
| | | ATCCGAGCAAGTTCTTTAGCCAGCTGAGCAGCGA |
| | | GCACGGCGGAGATGTTCAGAAGTGGCTGTCTAGC |
| | | CCATTTCCTAGCAGCAGCTTCAGCCCTGGTGGAC |
| | | TGGCCCCTGAGATTAGCCCTCTGGAAGTGCTGGA |
| | | ACGGGACAAAGTGACCCAGCTGCTTCTGCAGCAG |
| | | GACAAGGTGCCAGAACCTGCCAGCCTGTCCAGCA |
| | | ATCACAGCCTGACCAGCTGTTTTACAAACCAGGG |
| | | CTACTTCTTCTTCCATCTGCCTGACGCACTGGAA |
| | | ATCGAGGCCTGCCAGGTGTACTTCACCTACGATC |
| | | CCTACAGCGAAGAGGACCCCGATGAAGGTGTTGC |
| | | CGGTGCTCCTACCGGAAGCTCTCCTCAACCTCTG |
| | | CAACCACTGAGCGGCGAGGATGACGCCTACTGCA |
| | | CATTCCCCAGCAGGGACGATCTGCTGCTGTTCAG |
| | | CCCATCTCTGCTCGGCGGACCTTCTCCACCATCT |
| | | ACAGCTCCAGGTGGAAGCGGAGCCGGCGAGGAAA |
| | | GAATGCCACCTAGCCTGCAAGAGCGGGTGCCCAG |
| | | AGATTGGGACCCTCAACCACTTGGCCCTCCAACA |
| | | CCTGGCGTGCCAGACCTGGTTGATTTCCAGCCTC |
| | | CTCCAGAGCTGGTGCTGAGAGAAGCTGGCGAAGA |
| | | AGTGCCAGACGCCGGACCTAGAGAGGGCGTTAGC |
| | | TTTCCTTGGAGCAGACCTCCTGGACAAGGCGAGT |
| | | TCAGAGCCCTGAATGCCAGACTGCCCCTGAACAC |
| | | CGATGCCTACCTGTCTCTGCAAGAACTGCAGGGA |
| | | CAGGATCCCACACACCTGGTC |
| 43 | DNA Sequence Encoding | ATGGCTGCCCCTGCTCTGTCTTGGAGACTGCCTC |
| | DR626 | TGCTGATCCTGCTGCTGCCTCTGGCTACATCTTG |
| | | GGCCTCTGCCGCCGTGAATGGCACCAGCCAGTTC |
| | | ACCTGTTTCTACAACAGCCGGGCCAACATCAGCT |
| | | GCGTGTGGTCACAAGATGGCGCCCTGCAGGATAC |
| | | CAGCTGTCAGGTTCACGCCTGGCCTGATCGGAGA |
| | | AGATGGAACCAGACATGCGAGCTGCTGCCCGTGT |
| | | CTCAGGCTTCCTGGGCCTGTAATCTGATCCTGGG |
| | | AGCCCCTGACAGCCAGAAACTGACCACCGTGGAT |
| | | ATCGTGACCCTGCGGGTGCTGTGTAGAGAAGGCG |
| | | TTCGTTGGAGAGTGATGGCCATTCAGGACTTCAA |
| | | GCCCTTCGAGAACCTGCGGCTGATGGCCCCAATT |
| | | AGCCTGCAGGTTGTGCACGTGGAAACCCACAGAT |
| | | GCAATATCAGCTGGGAGATCAGCCAGGCCAGCGA |
| | | CTTCTTCGAGCGGCACCTGGAATTTGAGGCCCGG |
| | | ACACTGTCTCCAGGCCACACCTGGGAAGAAGCCC |
| | | CTCTGCTCACCCTGAAGCAGAAACAAGAGTGGAT |
| | | CTGCCTGGAAACCCTGACACCTGACACACAGTAC |
| | | GAGTTCCAAGTGCGCGTGAAGCCTCTCCAGGGCG |
| | | AGTTCACGACGTGGTCCCCTTGGAGTCAGCCCCT |
| | | GGCCTTTAGAACAAAGCCTGCCGCTCTGGGCAAA |
| | | GACACAATCCCTTGGCTGGGCCATCTGCTCGTTG |
| | | GACTGTCTGGCGCCTTCGGCTTCATCATCCTGGT |
| | | GTACCTGCTGATCAACTGCCGGAACACCGGACCT |
| | | TGGCTGAAGAAAGTGCTGAAGTGCAACACCCCTG |
| | | ATCCGAGCAAGTTCTTTAGCCAGCTGAGCAGCGA |
| | | GCACGGCGGAGATGTTCAGAAGTGGCTGTCTAGC |
| | | CCATTTCCTAGCAGCAGCTTCAGCCCTGGTGGAC |
| | | TGGCCCCTGAGATTAGCCCTCTGGAAGTGCTGGA |
| | | ACGGGACAAAGTGACCCAGCTGCTTCTGCAGCAG |
| | | GACAAGGTGCCAGAACCTGCCAGCCTGTCCAGCA |
| | | ATCACAGCCTGACCAGCTGTTTTACAAACCAGGG |
| | | CTACTTCTTCTTCCATCTGCCTGACGCACTGGAA |
| | | ATCGAGGCCTGCCAGGTGTACTTCACCTACGATC |
| | | CCTACAGCGAAGAGGACCCCGATGAAGGTGTTGC |
| | | CGGTGCTCCTACCGGAAGCTCTCCTCAACCTCTG |
| | | CAACCACTGAGCGGCGAGGATGACGCCTACTGCA |
| | | CATTCCCCAGCAGGGACGATCTGCTGCTGTTCAG |
| | | CCCATCTCTGCTCGGCGGACCTTCTCCACCATCT |
| | | ACAGCTCCAGGTGGAAGCGGAGCCGGCGAGGAAA |
| | | GAATGCCACCTAGCCTGCAAGAGCGGGTGCCCAG |
| | | AGATTGGGACCCTCAACCACTTGGCCCTCCAACA |
| | | CCTGGCGTGCCAGACCTGGTTGATTTCCAGCCTC |
| | | CTCCAGAGCTGGTGCTGAGAGAAGCTGGCGAAGA |
| | | AGTGCCAGACGCCGGACCTAGAGAGGGCGTTAGC |
| | | TTTCCTTGGAGCAGACCTCCTGGACAAGGCGAGT |
| | | TCAGAGCCCTGAATGCCAGACTGCCCCTGAACAC |
| | | CGATGCCTACCTGTCTCTGCAAGAACTGCAGGGA |
| | | CAGGATCCCACACACCTGGTCGGGAGCGGAGAAG |
| | | GCAGAGGAAGCTTGCTGACATGCGGCGACGTGGA |
| | | AGAGAACCCTGGTCCCATGGCTCTGCCTGTGACA |
| | | GCTCTGCTGCTGCCTCTGGCTCTGCTTCTGCATG |
| | | CCGCCCGTCCGGACGTGGTCATGACACAGAGCCC |
| | | TCTGAGCCTGCCTGTGACACCTGGCGAACCTGCC |
| | | AGCATCAGCTGTAGAAGCAGCCAGAGCCTGGTGC |
| | | ACAGCAACGCCAACACATACCTGCACTGGTATCT |
| | | GCAGAAGCCCGGCCAGTCTCCTCAGCTGCTGATC |
| | | TACAAGGTGTCCAACAGATTCAGCGGCGTGCCCG |
| | | ACAGATTCTCTGGCTCTGGATCTGGCACCGACTT |
| | | CACCCTGAAGATCTCCAGAGTGGAAGCCGAGGAC |
| | | GTGGGCGTGTACTACTGCAGCCAGAACACACACG |
| | | TGCCACCTACATTCGGCCAGGGCACCAAGCTGGA |
| | | AATCAAGAGGGGATCCACAAGCGGCTCTGGCAAG |
| | | CCTGGAAGCGGAGAGGGATCTACCAAAGGCCAGG |
| | | TGCAGCTGGTTCAGTCTGGCGCCGAAGTGAAGAA |
| | | ACCTGGCGCCTCTGTGAAGGTGTCCTGCAAGGCA |
| | | AGCGGCTACACATTCACCGACTACGAGATGCACT |
| | | GGGTCCGACAGGCTCCAGGACAGGGACTTGAATG |
| | | GATGGGAGCCCTAGATCCTAAGACCGGCGATACA |
| | | GCCTACAGCCAGAAATTCAAGGGAAGAGTGACCC |
| | | TGACCGCCGACGAGAGCACATCTACCGCCTACAT |
| | | GGAACTGAGCAGCCTGAGAAGCGAGGACACCGCC |
| | | GTGTATTACTGTACCAGATTCTACTCCTACACCT |
| | | ACTGGGGCCAGGGAACCCTGGTCACAGTCTCTTC |
| | | TGCGGCCGCTATCGAAGTGATGTACCCTCCTCCT |
| | | TACCTGGACAACGAGAAGTCCAACGGCACCATCA |
| | | TCCACGTGAAGGGCAAGCACCTGTGTCCTTCTCC |
| | | ACTGTTCCCCGGACCTAGCAAGCCTTTCTGGGTG |
| | | CTCGTTGTTGTTGGCGGCGTGCTGGCCTGTTACT |
| | | CTCTGCTGGTTACCGTGGCCTTCATCATCTTTTG |
| | | GGTCCGATCCAAGCGGAGCCGGCTGCTGCACAGC |
| | | GACTACATGAACATGACCCCTAGACGGCCCGGAC |
| | | CAACCAGAAAGCACTACCAGCCTTACGCTCCTCC |
| | | TCGGGATTTCGCCGCCTACAGATCCAGAGTGAAG |
| | | TTCTCCAGAAGCGCCGACGCTCCTGCCTATCAGC |
| | | AGGGACAGAATCAGCTGTACAACGAGCTGAACCT |
| | | GGGGCGCAGAGAAGAGTACGACGTGCTGGATAAG |
| | | CGGAGAGGCAGAGATCCTGAGATGGGCGGCAAGC |
| | | CCAGACGGAAGAATCCTCAAGAGGGCCTGTATAA |
| | | TGAGCTGCAGAAAGACAAGATGGCCGAGGCCTAC |
| | | AGCGAGATCGGAATGAAGGGCGAACGCAGAAGAG |
| | | GCAAGGGCCACGATGGACTGTACCAGGGACTGAG |
| | | CACCGCCACCAAGGATACCTATGACGCCCTGCAC |
| | | ATGCAGGCTCTGCCACCTAGA |
| 44 | DR1581 DNA SEQUENCE | ATGGCTCTGCCTGTGACAGCTCTGCTGCTGCCTC |
| | | TGGCTCTGCTTCTGCATGCCGCCAGACCTGACGT |
| | | GGTCATGACACAGAGCCCTCTGAGCCTGCCTGTG |
| | | ACACCTGGCGAACCTGCCAGCATCAGCTGTAGAA |
| | | GCAGCCAGAGCCTGGTGCACAGCAACGCCAACAC |
| | | ATACCTGCACTGGTATCTGCAGAAGCCCGGCCAG |
| | | TCTCCTCAGCTGCTGATCTACAAGGTGTCCAACA |
| | | GATTCAGCGGCGTGCCCGACAGATTCTCTGGCTC |
| | | TGGATCTGGCACCGACTTCACCCTGAAGATCTCC |
| | | AGAGTGGAAGCCGAGGACGTGGGCGTGTACTACT |
| | | GCAGCCAGAACACACACGTGCCACCTACATTCGG |
| | | CCAGGGCACCAAGCTGGAAATCAAGAGGGGATCC |
| | | ACAAGCGGCTCTGGCAAGCCTGGAAGCGGAGAGG |
| | | GATCTACCAAAGGCCAGGTGCAGCTGGTTCAGTC |
| | | TGGCGCCGAAGTGAAGAAACCTGGCGCCTCTGTG |
| | | AAGGTGTCCTGCAAGGCAAGCGGCTACACATTCA |
| | | CCGACTACGAGATGCACTGGGTCCGACAGGCTCC |
| | | AGGACAGGGACTTGAATGGATGGGAGCCCTAGAT |
| | | CCTAAGACCGGCGATACAGCCTACAGCCAGAAAT |
| | | TCAAGGGAAGAGTGACCCTGACCGCCGACGAGAG |
| | | CACATCTACCGCCTACATGGAACTGAGCAGCCTG |
| | | AGAAGCGAGGACACCGCCGTGTATTACTGTACCA |
| | | GATTCTACTCCTACACCTACTGGGGCCAGGGAAC |
| | | CCTGGTCACAGTCTCGAGCACTACGACCCCGGCA |
| | | CCTCGCCCTCCCACCCCTGCGCCTACTATTGCCT |
| | | CACAGCCGCTGTCCCTGCGCCCTGAGGCCTGTCG |
| | | CCCAGCTGCCGGAGGGGCCGTGCACACACGCGGA |
| | | CTGGATTTCGCCTGCGATATTTATATCTGGGCTC |
| | | CGCTCGCCGGAACCTGTGGTGTGCTCTTGCTGTC |
| | | CCTCGTAATCACCCTTTACTGTAAGAGGGGACGG |
| | | AAAAAGTTGCTCTACATTTTCAAGCAGCCATTTA |
| | | TGCGGCCCGTTCAGACAACCCAGGAAGAGGACGG |
| | | TTGCAGTTGCAGATTTCCCGAGGAAGAGGAAGGA |
| | | GGTTGCGAGTTGAGAGTGAAGTTCTCCAGAAGCG |
| | | CCGACGCTCCTGCCTATCAGCAGGGACAGAATCA |
| | | GCTGTACAACGAGCTGAACCTGGGGCGCAGAGAA |
| | | GAGTACGACGTGCTGGATAAGCGGAGAGGCAGAG |
| | | ATCCTGAGATGGGCGGCAAGCCCAGACGGAAGAA |
| | | TCCTCAAGAGGGCCTGTATAATGAGCTGCAGAAA |
| | | GACAAGATGGCCGAGGCCTACAGCGAGATCGGAA |
| | | TGAAGGGCGAACGCAGAAGAGGCAAGGGCCACGA |
| | | TGGACTGTACCAGGGACTGAGCACCGCCACCAAG |
| | | GATACCTATGACGCCCTGCACATGCAGGCTCTGC |
| | | CACCTAGAGGGTCCGGAGAAGGCAGAGGATCTCT |
| | | GCTGACATGCGGCGACGTGGAAGAGAACCCTGGG |
| | | CCCATGGCTGCCCCTGCTCTGTCTTGGAGACTGC |
| | | CTCTGCTGATCCTGCTGCTGCCTCTGGCTACATC |
| | | TTGGGCCTCTGCCGCCGTGAATGGCACCAGCCAG |
| | | TTCACCTGTTTCTACAACAGCCGGGCCAACATCA |
| | | GCTGCGTGTGGTCACAAGATGGCGCCCTGCAGGA |
| | | TACCAGCTGTCAGGTTCACGCCTGGCCTGATCGG |
| | | AGAAGATGGAACCAGACATGCGAGCTGCTGCCCG |
| | | TGTCTCAGGCTTCCTGGGCCTGTAATCTGATCCT |
| | | GGGAGCCCCTGACAGCCAGAAACTGACCACCGTG |
| | | GATATCGTGACCCTGCGGGTGCTGTGTAGAGAAG |
| | | GCGTTCGTTGGAGAGTGATGGCCATTCAGGACTT |
| | | CAAGCCCTTCGAGAACCTGCGGCTGATGGCCCCA |
| | | ATTAGCCTGCAGGTTGTGCACGTGGAAACCCACA |
| | | GATGCAATATCAGCTGGGAGATCAGCCAGGCCAG |
| | | CGACTTCTTCGAGCGGCACCTGGAATTTGAGGCC |
| | | CGGACACTGTCTCCAGGCCACACCTGGGAAGAAG |
| | | CCCCTCTGCTCACCCTGAAGCAGAAACAAGAGTG |
| | | GATCTGCCTGGAAACCCTGACACCTGACACACAG |
| | | TACGAGTTCCAAGTGCGCGTGAAGCCTCTCCAGG |
| | | GCGAGTTCACGACGTGGTCCCCTTGGAGTCAGCC |
| | | CCTGGCCTTTAGAACAAAGCCTGCCGCTCTGGGC |
| | | AAAGACACAATCCCTTGGCTGGGCCATCTGCTCG |
| | | TTGGACTGTCTGGCGCCTTCGGCTTCATCATCCT |
| | | GGTGTACCTGCTGATCAACTGCCGGAACACCGGA |
| | | CCTTGGCTGAAGAAAGTGCTGAAGTGCAACACCC |
| | | CTGATCCGAGCAAGTTCTTTAGCCAGCTGAGCAG |
| | | CGAGCACGGCGGAGATGTTCAGAAGTGGCTGTCT |
| | | AGCCCATTTCCTAGCAGCAGCTTCAGCCCTGGTG |
| | | GACTGGCCCCTGAGATTAGCCCTCTGGAAGTGCT |
| | | GGAACGGGACAAAGTGACCCAGCTGCTTCTGCAG |
| | | CAGGACAAGGTGCCAGAACCTGCCAGCCTGTCCA |
| | | GCAATCACAGCCTGACCAGCTGTTTTACAAACCA |
| | | GGGCTACTTCTTCTTCCATCTGCCTGACGCACTG |
| | | GAAATCGAGGCCTGCCAGGTGTACTTCACCTACG |
| | | ATCCCTACAGCGAAGAGGACCCCGATGAAGGTGT |
| | | TGCCGGTGCTCCTACCGGAAGCTCTCCTCAACCT |
| | | CTGCAACCACTGAGCGGCGAGGATGACGCCTACT |
| | | GCACATTCCCCAGCAGGGACGATCTGCTGCTGTT |
| | | CAGCCCATCTCTGCTCGGCGGACCTTCTCCACCA |
| | | TCTACAGCTCCAGGTGGAAGCGGAGCCGGCGAGG |
| | | AAAGAATGCCACCTAGCCTGCAAGAGCGGGTGCC |
| | | CAGAGATTGGGACCCTCAACCACTTGGCCCTCCA |
| | | ACACCTGGCGTGCCAGACCTGGTTGATTTCCAGC |
| | | CTCCTCCAGAGCTGGTGCTGAGAGAAGCTGGCGA |
| | | AGAAGTGCCAGACGCCGGACCTAGAGAGGGCGTT |
| | | AGCTTTCCTTGGAGCAGACCTCCTGGACAAGGCG |
| | | AGTTCAGAGCCCTGAATGCCAGACTGCCCCTGAA |
| | | CACCGATGCCTACCTGTCTCTGCAAGAACTGCAG |
| | | GGACAGGATCCCACACACCTGGTC |
| 45 | DNA Sequence DR628 CAR | ATGGCTCTGCCTGTGACAGCTCTGCTGCTGCCTC |
| | | TGGCTCTGCTTCTGCATGCCGCTAGACCTGACGT |
| | | GGTTATGACACAGAGCCCTCTGAGCCTGCCTGTG |
| | | ACACCTGGCGAACCTGCCAGCATCAGCTGTAGAA |
| | | GCAGCCAGAGCCTGGTGCACAGCAACGCCAACAC |
| | | ATACCTGCACTGGTATCTGCAGAAGCCCGGCCAG |
| | | TCTCCTCAGCTGCTGATCTACAAGGTGTCCAACA |
| | | GATTCAGCGGCGTGCCCGACAGATTCTCTGGCTC |
| | | TGGATCTGGCACCGACTTCACCCTGAAGATCTCC |
| | | AGAGTGGAAGCCGAGGACGTGGGCGTGTACTACT |
| | | GCAGCCAGAACACACACGTGCCACCTACATTCGG |
| | | CCAGGGCACCAAGCTGGAAATCAAGAGGGGATCC |
| | | ACAAGCGGCTCTGGCAAGCCTGGAAGCGGAGAGG |
| | | GATCTACCAAAGGCCAGGTGCAGCTGGTTCAGTC |
| | | TGGCGCCGAAGTGAAGAAACCTGGCGCCTCTGTG |
| | | AAGGTGTCCTGCAAGGCAAGCGGCTACACATTCA |
| | | CCGACTACGAGATGCACTGGGTCCGACAGGCTCC |
| | | AGGACAGGGACTTGAATGGATGGGAGCCCTAGAT |
| | | CCTAAGACCGGCGATACAGCCTACAGCCAGAAAT |
| | | TCAAGGGAAGAGTGACCCTGACCGCCGACGAGAG |
| | | CACATCTACCGCCTACATGGAACTGAGCAGCCTG |
| | | AGAAGCGAGGACACCGCCGTGTATTACTGTACCA |
| | | GATTCTACTCCTACACCTACTGGGGCCAGGGAAC |
| | | CCTGGTCACAGTCTCTTCTACAACAACCCCTGCT |
| | | CCTAGACCTCCTACACCAGCTCCTACAATCGCCA |
| | | GCCAGCCTCTGTCTCTGAGGCCAGAGGCTTGTAG |
| | | ACCTGCTGCTGGCGGAGCCGTGCATACAAGAGGA |
| | | CTGGATTTCGCCTGCGACATCTATATCTGGGCCC |
| | | CTCTGGCCGGAACATGTGGCGTTTTGCTGCTGAG |
| | | CCTGGTCATCACCAAGCGGGGCAGAAAGAAGCTG |
| | | CTGTACATCTTCAAGCAGCCCTTCATGCGGCCCG |
| | | TGCAGACCACACAAGAGGAAGATGGCTGCTCCTG |
| | | CAGATTCCCCGAGGAAGAAGAAGGCGGCTGCGAG |
| | | CTGAGAGTGAAGTTCTCCAGATCCGCCGACGCTC |
| | | CTGCCTATCAGCAGGGACAGAATCAGCTGTACAA |
| | | CGAGCTGAACCTGGGGCGCAGAGAAGAGTACGAC |
| | | GTGCTGGATAAGCGGAGAGGCAGAGATCCTGAGA |
| | | TGGGCGGAAAGCCCCAGCGGAGAAAGAATCCTCA |
| | | AGAGGGCCTGTATAATGAGCTGCAGAAAGACAAG |
| | | ATGGCCGAGGCCTACAGCGAGATCGGAATGAAGG |
| | | GCGAACGCAGAAGAGGCAAGGGCCACGATGGACT |
| | | GTACCAGGGACTGAGCACCGCCACCAAGGATACC |
| | | TATGACGCCCTGCACATGCAGGCTCTGCCACCTA |
| | | GA |
| 46 | DNA Sequence Encoding | ATGGCTCTGCCGGTTACAGCCCTGCTGCTTCCTC |
| | DR625 | TCGCCCTCCTTCTTCATGCAGCGCGACCGGATGT |
| | (codon optimized) | CGTGATGACCCAGAGCCCCCTGTCTCTGCCCGTC |
| | | ACTCCAGGTGAACCGGCATCAATATCATGTCGAA |
| | | GTAGTCAATCACTCGTTCATTCAAACGCAAATAC |
| | | CTATCTCCACTGGTACCTGCAGAAGCCCGGACAA |
| | | TCACCTCAGTTGCTGATCTACAAGGTATCTAACC |
| | | GCTTCTCAGGCGTTCCAGATAGATTCTCTGGGTC |
| | | TGGTTCCGGGACAGACTTTACGCTCAAAATTAGC |
| | | CGAGTTGAGGCTGAGGACGTAGGGGTGTATTATT |
| | | GTTCCCAAAATACTCATGTTCCGCCCACCTTCGG |
| | | GCAGGGCACCAAACTTGAAATAAAGCGCGGCAGC |
| | | ACTTCAGGAAGCGGCAAACCTGGCTCTGGTGAGG |
| | | GAAGCACGAAGGGCCAGGTACAACTCGTACAATC |
| | | AGGAGCTGAAGTTAAAAAGCCCGGGGCGTCAGTC |
| | | AAAGTGTCCTGCAAGGCCAGTGGCTATACGTTTA |
| | | CTGACTATGAGATGCATTGGGTGCGCCAGGCTCC |
| | | GGGTCAAGGGCTCGAGTGGATGGGAGCACTTGAT |
| | | CCCAAGACGGGTGACACTGCTTACTCTCAGAAAT |
| | | TTAAAGGGCGGGTTACACTCACGGCGGACGAGTC |
| | | CACATCAACGGCGTATATGGAGTTGAGTTCACTG |
| | | CGAAGCGAAGATACGGCTGTCTACTATTGCACCC |
| | | GATTTTATTCATACACGTATTGGGGTCAAGGTAC |
| | | CCTCGTGACTGTTTCATCAGCTGCCGCGATCGAG |
| | | GTGATGTATCCTCCGCCCTACTTGGACAACGAAA |
| | | AGTCCAACGGCACCATCATTCATGTGAAAGGCAA |
| | | ACACCTCTGCCCTTCCCCCCTTTTCCCCGGCCCA |
| | | AGCAAGCCCTTCTGGGTCTTGGTAGTAGTGGGTG |
| | | GGGTGCTCGCGTGCTACTCTCTGCTGGTCACAGT |
| | | GGCCTTCATTATCTTTTGGGTCCGGTCAAAACGC |
| | | TCTCGACTGCTTCACTCTGATTACATGAATATGA |
| | | CTCCCAGACGGCCAGGGCCTACGAGAAAGCATTA |
| | | CCAACCTTATGCACCACCTCGGGATTTCGCAGCA |
| | | TATCGATCCAGGGTCAAGTTCTCACGATCAGCAG |
| | | ACGCCCCGGCCTACCAACAAGGCCAAAATCAACT |
| | | GTATAATGAACTGAACCTTGGGCGGCGAGAGGAG |
| | | TATGACGTTCTCGATAAACGCAGAGGGCGCGACC |
| | | CAGAGATGGGTGGGAAACCGCGGCGCAAAAACCC |
| | | CCAGGAAGGACTCTATAATGAGCTCCAAAAGGAT |
| | | AAAATGGCTGAAGCTTACAGTGAAATCGGAATGA |
| | | AGGGTGAACGGAGGCGAGGGAAGGGACACGATGG |
| | | CTTGTATCAGGGACTTTCCACAGCTACAAAGGAT |
| | | ACTTACGACGCCTTGCACATGCAGGCGCTTCCTC |
| | | CTCGGGGGAGTGGAGAGGGCCGCGGATCACTTCT |
| | | CACATGCGGCGACGTAGAGGAAAATCCTGGCCCC |
| | | ATGGCAGCACCGGCGCTTTCATGGCGGCTGCCGC |
| | | TGTTGATACTTCTTTTGCCTCTGGCAACATCATG |
| | | GGCTTCCGCCGCCGTAAATGGGACCTCCCAATTT |
| | | ACTTGCTTCTACAACAGTCGCGCGAACATCTCCT |
| | | GCGTCTGGAGCCAGGACGGCGCTCTCCAAGACAC |
| | | GAGCTGTCAAGTACATGCCTGGCCGGATAGACGC |
| | | AGATGGAATCAGACCTGCGAGTTGCTCCCTGTAT |
| | | CCCAAGCTTCATGGGCTTGCAATCTCATCTTGGG |
| | | CGCCCCAGATAGCCAAAAATTGACTACGGTAGAT |
| | | ATTGTCACTTTGAGAGTCCTCTGCCGCGAAGGCG |
| | | TCAGATGGAGAGTAATGGCAATTCAAGATTTTAA |
| | | ACCATTTGAGAATCTGCGACTTATGGCCCCTATC |
| | | AGTTTGCAAGTAGTACATGTGGAAACGCATCGAT |
| | | GCAATATATCATGGGAGATAAGCCAGGCGAGTGA |
| | | TTTCTTCGAAAGGCACCTCGAGTTTGAGGCCCGA |
| | | ACTTTGAGCCCAGGTCACACTTGGGAAGAAGCAC |
| | | CCTTGCTCACTCTTAAACAGAAACAGGAGTGGAT |
| | | ATGTTTGGAGACACTTACTCCGGACACGCAGTAT |
| | | GAGTTCCAAGTTAGAGTGAAACCCCTGCAAGGAG |
| | | AATTCACCACTTGGTCTCCATGGAGTCAACCATT |
| | | GGCTTTTCGCACTAAACCCGCGGCGCTTGGAAAG |
| | | GACACTATACCGTGGCTTGGACATTTGCTCGTCG |
| | | GTCTGAGCGGAGCTTTCGGGTTTATTATATTGGT |
| | | CTATTTGCTCATTAATTGCAGGAATACAGGCCCG |
| | | TGGCTGAAGAAGGTTCTCAAATGTAATACACCCG |
| | | ACCCATCTAAGTTTTTCTCTCAACTCTCCTCTGA |
| | | GCACGGAGGAGACGTCCAAAAGTGGCTTTCAAGC |
| | | CCCTTCCCGAGTAGTTCCTTTAGTCCGGGTGGAC |
| | | TCGCGCCGGAGATTAGTCCCCTGGAAGTATTGGA |
| | | ACGAGACAAGGTTACTCAGCTCCTTCTGCAGCAA |
| | | GACAAGGTTCCTGAACCCGCTTCCTTGTCCAGTA |
| | | ATCATTCACTGACATCTTGCTTTACCAACCAAGG |
| | | ATATTTTTTTTTCCATCTTCCTGACGCCCTGGAA |
| | | ATTGAGGCTTGCCAAGTGTACTTTACGTACGACC |
| | | CTTATTCCGAAGAAGACCCAGACGAGGGCGTAGC |
| | | GGGTGCACCAACTGGGTCCAGCCCTCAACCGCTT |
| | | CAGCCGCTTAGTGGGGAAGACGATGCATATTGTA |
| | | CTTTCCCCAGTAGGGACGATCTTCTTCTCTTTTC |
| | | CCCAAGTCTGCTCGGGGGCCCGAGCCCTCCATCA |
| | | ACCGCCCCAGGCGGTTCAGGCGCCGGCGAAGAGA |
| | | GAATGCCTCCAAGCCTTCAAGAAAGAGTTCCACG |
| | | GGACTGGGATCCTCAGCCACTTGGTCCACCGACT |
| | | CCCGGTGTTCCTGATCTGGTTGACTTCCAGCCTC |
| | | CACCAGAGCTTGTGCTTAGGGAGGCAGGCGAAGA |
| | | AGTGCCCGACGCTGGGCCAAGGGAGGGAGTTAGT |
| | | TTCCCGTGGTCTAGACCGCCTGGCCAGGGCGAGT |
| | | TCAGAGCTTTGAACGCGCGGCTCCCTTTGAATAC |
| | | CGACGCTTACTTGTCATTGCAAGAGCTGCAAGGG |
| | | CAAGACCCGACACACTTGGTGTGATAA |
| 47 | Modified AllStem vector used | TTTGGGGTTGCTCTGGAAAACTCATTTGCACCAC |
| | for construction of DR625, | TGCTGTGCCTTGGAATGCTAGTTGGAGTAATAAA |
| | DR626 and DR628 CAR T cells | TCTCTGGAACAGATTTGGAATCACACGACCTGGA |
| | used in studies provided | TGGAGTGGGACAGAGAAATTAACAATTACACAAG |
| | | CTTAATACACTCCTTAATTGAAGAATCGCAAAAC |
| | | CAGCAAGAAAAGAATGAACAAGAATTATTGGAAT |
| | | TAGATAAATGGGCAAGTTTGTGGAATTGGTTTAA |
| | | CATAACAAATTGGCTGTGGTATATAAAATTATTC |
| | | ATAATGATAGTAGGAGGCTTGGTAGGTTTAAGAA |
| | | TAGTTTTTGCTGTACTTTCTATAGTGAATAGAGT |
| | | TAGGCAGGGATATTCACCATTATCGTTTCAGACC |
| | | CACCTCCCAACCCCGAGGGGACCCGACAGGCCCG |
| | | AAGGAATAGAAGAAGAAGGTGGAGAGAGAGACAG |
| | | AGACAGATCCATTCGATTAGTGAACGGATCGGCA |
| | | CTGCGTGCGCCAATTCTGCAGACAAATGGCAGTA |
| | | TTCATCCACAATTTTAAAAGAAAAGGGGGGATTG |
| | | GGGGGTACAGTGCAGGGGAAAGAATAGTAGACAT |
| | | AATAGCAACAGACATACAAACTAAAGAATTACAA |
| | | AAACAAATTACAAAAATTCAAAATTTTCGGGTTT |
| | | ATTACAGGGACAGCAGAGATCCAGTTTGGTTAAT |
| | | TAAAAGGATCTGCGATCGCTCCGGTGCCCGTCAG |
| | | TGGGCAGAGCGCACATCGCCCACAGTCCCCGAGA |
| | | AGTTGGGGGGAGGGGTCGGCAATTGAACGGGTGC |
| | | CTAGAGAAGGTGGCGCGGGGTAAACTGGGAAAGT |
| | | GATGTCGTGTACTGGCTCCGCCTTTTTCCCGAGG |
| | | GTGGGGGAGAACCGTATATAAGTGCAGTAGTCGC |
| | | CGTGAACGTTCTTTTTCGCAACGGGTTTGCCGCC |
| | | AGAACACAGCTGAAGCTTCGAGGGGCTCGCATCT |
| | | CTCCTTCACGCGCCCGCCGCCCTACCTGAGGCCG |
| | | CCATCCACGCCGGTTGAGTCGCGTTCTGCCGCCT |
| | | CCCGCCTGTGGTGCCTCCTGAACTGCGTCCGCCG |
| | | TCTAGGTAAGTTTAAAGCTCAGGTCGAGACCGGG |
| | | CCTTTGTCCGGCGCTCCCTTGGAGCCTACCTAGA |
| | | CTCAGCCGGCTCTCCACGCTTTGCCTGACCCTGC |
| | | TTGCTCAACTCTACGTCTTTGTTTCGTTTTCTGT |
| | | TCTGCGCCGTTACAGATCCAAGCTGTGACCGGCG |
| | | CCTACGAATTCGAATTTAAATCGGATCCGACCGG |
| | | TGGCGCGCCCTCGAGGGTACCGCTAGCACGCGTT |
| | | AAGTCGACAATCAACCTCTGGATTACAAAATTTG |
| | | TGAAAGATTGACTGGTATTCTTAACTATGTTGCT |
| | | CCTTTTACGCTATGTGGATACGCTGCTTTAATGC |
| | | CTTTGTATCATGCTATTGCTTCCCGTATGGCTTT |
| | | CATTTTCTCCTCCTTGTATAAATCCTGGTTGCTG |
| | | TCTCTTTATGAGGAGTTGTGGCCCGTTGTCAGGC |
| | | AACGTGGCGTGGTGTGCACTGTGTTTGCTGACGC |
| | | AACCCCCACTGGTTGGGGCATTGCCACCACCTGT |
| | | CAGCTCCTTTCCGGGACTTTCGCTTTCCGCCTCC |
| | | CTATTGCCACGGCGGAACTCATCGCCGCCTGCCT |
| | | TGCCCGCTGCTGGACAGGGGCTCGGCTGTTGGGC |
| | | ACTGACAATTCCGTGGTGTTGTCGGGGAAATCAT |
| | | CGTCCTTTCCTTGGCTGCTCGCCTGTGTTGCCAC |
| | | CTGGATTCTGCGCGGGACGTCCTTCTGCTACGTC |
| | | CCTTCGGCCCTCAATCCAGCGGACCTTCCTTCCC |
| | | GCGGCCTGCTGCCGGCTCTGCGGCCTCTTCCGCG |
| | | TCTTCGCCTTCGCCCTCAGACGAGTCGGATCTCC |
| | | CTTTGGGCCGCCTCCCCGCGTCGACTTTAAGACC |
| | | AATGACTTACAAGGCAGCTGTAGATCTTAGCCAC |
| | | TTTTTAAAAGAAAAGGGGGGACTGGAAGGGCTAA |
| | | TTCACTCCCAACGAAGACAAGATCTGCTTTTTGC |
| | | TTGTACTGGGTCTCTCTGGTTAGACCAGATCTGA |
| | | GCCTGGGAGCTCTCTGGCTAACTAGGGAACCCAC |
| | | TGCTTAAGCCTCAATAAAGCTTGCCTTGAGTGCT |
| | | TCAAGTAGTGTGTGCCCGTCTGTTGTGTGACTCT |
| | | GGTAACTAGAGATCCCTCAGACCCTTTTAGTCAG |
| | | TGTGGAAAATCTCTAGCAGGGCCCGTTTAAACCC |
| | | GCTGATCAGCCTCGACTGTGCCTTCTAGTTGCCA |
| | | GCCATCTGTTGTTTGCCCCTCCCCCGTGCCTTCC |
| | | TTGACCCTGGAAGGTGCCACTCCCACTGTCCTTT |
| | | CCTAATAAAATGAGGAAATTGCATCGCATTGTCT |
| | | GAGTAGGTGTCATTCTATTCTGGGGGGTGGGGTG |
| | | GGGCAGGACAGCAAGGGGGAGGATTGGGAAGACA |
| | | ATAGCAGGCATGCTGGGGATGCGGTGGGCTCTAT |
| | | GGCTTCTGAGGCGGAAAGAACCAGCTGGGGCTCT |
| | | AGGGGGTATCCCCACGCGCCCTGTAGCGGCGCAT |
| | | TAAGCGCGGCGGGTGTGGTGGTTACGCGCAGCGT |
| | | GACCGCTACACTTGCCAGCGCCCTAGCGCCCGCT |
| | | CCTTTCGCTTTCTTCCCTTCCTTTCTCGCCACGT |
| | | TCGCCGGCTTTCCCCGTCAAGCTCTAAATCGGGG |
| | | GCTCCCTTTAGGGTTCCGATTTAGTGCTTTACGG |
| | | CACCTCGACCCCAAAAAACTTGATTAGGGTGATG |
| | | GTTCACGTAGTGGGCCATCGCCCTGATAGACGGT |
| | | TTTTCGCCCTTTGACGTTGGAGTCCACGTTCTTT |
| | | AATAGTGGACTCTTGTTCCAAACTGGAACAACAC |
| | | TCAACCCTATCTCGGTCTATTCTTTTGATTTATA |
| | | AGGGATTTTGCCGATTTCGGCCTATTGGTTAAAA |
| | | AATGAGCTGATTTAACAAAAATTTAACGCGAATT |
| | | AATTCTGTGGAATGTGTGTCAGTTAGGGTGTGGA |
| | | AAGTCCCCAGGCTCCCCAGCAGGCAGAAGTATGC |
| | | AAAGCATGCATCTCAATTAGTCAGCAACCAGGTG |
| | | TGGAAAGTCCCCAGGCTCCCCAGCAGGCAGAAGT |
| | | ATGCAAAGCATGCATCTCAATTAGTCAGCAACCA |
| | | TAGTCCCGCCCCTAACTCCGCCCATCCCGCCCCT |
| | | AACTCCGCCCAGTTCCGCCCATTCTCCGCCCCAT |
| | | GGCTGACTAATTTTTTTTATTTATGCAGAGGCCG |
| | | AGGCCGCCTCTGCCTCTGAGCTATTCCAGAAGTA |
| | | GTGAGGAGGCTTTTTTGGAGGCCTAGGCTTTTGC |
| | | AAAAAGCTCCCGGGAGCTTGTATATCCATTTTCG |
| | | GATCTGATCAGCACGTGTTGACAATTAATCATCG |
| | | GCATAGTATATCGGCATAGTATAATACGACAAGG |
| | | TGAGGAACTAAACCATGGCCAAGTTGACCAGTGC |
| | | CGTTCCGGTGCTCACCGCGCGCGACGTCGCCGGA |
| | | GCGGTCGAGTTCTGGACCGACCGGCTCGGGTTCT |
| | | CCCGGGACTTCGTGGAGGACGACTTCGCCGGTGT |
| | | GGTCCGGGACGACGTGACCCTGTTCATCAGCGCG |
| | | GTCCAGGACCAGGTGGTGCCGGACAACACCCTGG |
| | | CCTGGGTGTGGGTGCGCGGCCTGGACGAGCTGTA |
| | | CGCCGAGTGGTCGGAGGTCGTGTCCACGAACTTC |
| | | CGGGACGCCTCCGGGCCGGCCATGACCGAGATCG |
| | | GCGAGCAGCCGTGGGGGCGGGAGTTCGCCCTGCG |
| | | CGACCCGGCCGGCAACTGCGTGCACTTCGTGGCC |
| | | GAGGAGCAGGACTGACACGTGCTACGAGATTTCG |
| | | ATTCCACCGCCGCCTTCTATGAAAGGTTGGGCTT |
| | | CGGAATCGTTTTCCGGGACGCCGGCTGGATGATC |
| | | CTCCAGCGCGGGGATCTCATGCTGGAGTTCTTCG |
| | | CCCACCCCAACTTGTTTATTGCAGCTTATAATGG |
| | | TTACAAATAAAGCAATAGCATCACAAATTTCACA |
| | | AATAAAGCATTTTTTTCACTGCATTCTAGTTGTG |
| | | GTTTGTCCAAACTCATCAATGTATCTTATCATGT |
| | | CTGTATACCGTCGACCTCTAGCTAGAGCTTGGCG |
| | | TAATCATGGTCATAGCTGTTTCCTGTGTGAAATT |
| | | GTTATCCGCTCACAATTCCACACAACATACGAGC |
| | | CGGAAGCATAAAGTGTAAAGCCTGGGGTGCCTAA |
| | | TGAGTGAGCTAACTCACATTAATTGCGTTGCGCT |
| | | CACTGCCCGCTTTCCAGTCGGGAAACCTGTCGTG |
| | | CCAGCTGCATTAATGAATCGGCCAACGCGCGGGG |
| | | AGAGGCGGTTTGCGTATTGGGCGCTCTTCCGCTT |
| | | CCTCGCTCACTGACTCGCTGCGCTCGGTCGTTCG |
| | | GCTGCGGCGAGCGGTATCAGCTCACTCAAAGGCG |
| | | GTAATACGGTTATCCACAGAATCAGGGGATAACG |
| | | CAGGAAAGAACATGTGAGCAAAAGGCCAGCAAAA |
| | | GGCCAGGAACCGTAAAAAGGCCGCGTTGCTGGCG |
| | | TTTTTCCATAGGCTCCGCCCCCCTGACGAGCATC |
| | | ACAAAAATCGACGCTCAAGTCAGAGGTGGCGAAA |
| | | CCCGACAGGACTATAAAGATACCAGGCGTTTCCC |
| | | CCTGGAAGCTCCCTCGTGCGCTCTCCTGTTCCGA |
| | | CCCTGCCGCTTACCGGATACCTGTCCGCCTTTCT |
| | | CCCTTCGGGAAGCGTGGCGCTTTCTCATAGCTCA |
| | | CGCTGTAGGTATCTCAGTTCGGTGTAGGTCGTTC |
| | | GCTCCAAGCTGGGCTGTGTGCACGAACCCCCCGT |
| | | TCAGCCCGACCGCTGCGCCTTATCCGGTAACTAT |
| | | CGTCTTGAGTCCAACCCGGTAAGACACGACTTAT |
| | | CGCCACTGGCAGCAGCCACTGGTAACAGGATTAG |
| | | CAGAGCGAGGTATGTAGGCGGTGCTACAGAGTTC |
| | | TTGAAGTGGTGGCCTAACTACGGCTACACTAGAA |
| | | GAACAGTATTTGGTATCTGCGCTCTGCTGAAGCC |
| | | AGTTACCTTCGGAAAAAGAGTTGGTAGCTCTTGA |
| | | TCCGGCAAACAAACCACCGCTGGTAGCGGTGGTT |
| | | TTTTTGTTTGCAAGCAGCAGATTACGCGCAGAAA |
| | | AAAAGGATCTCAAGAAGATCCTTTGATCTTTTCT |
| | | ACGGGGTCTGACGCTCAGTGGAACGAAAACTCAC |
| | | GTTAAGGGATTTTGGTCATGAGATTATCAAAAAG |
| | | GATCTTCACCTAGATCCTTTTAAATTAAAAATGA |
| | | AGTTTTAAATCAATCTAAAGTATATATGAGTAAA |
| | | CTTGGTCTGACAGTTACCAATGCTTAATCAGTGA |
| | | GGCACCTATCTCAGCGATCTGTCTATTTCGTTCA |
| | | TCCATAGTTGCCTGACTCCCCGTCGTGTAGATAA |
| | | CTACGATACGGGAGGGCTTACCATCTGGCCCCAG |
| | | TGCTGCAATGATACCGCGAGACCCACGCTCACCG |
| | | GCTCCAGATTTATCAGCAATAAACCAGCCAGCCG |
| | | GAAGGGCCGAGCGCAGAAGTGGTCCTGCAACTTT |
| | | ATCCGCCTCCATCCAGTCTATTAATTGTTGCCGG |
| | | GAAGCTAGAGTAAGTAGTTCGCCAGTTAATAGTT |
| | | TGCGCAACGTTGTTGCCATTGCTACAGGCATCGT |
| | | GGTGTCACGCTCGTCGTTTGGTATGGCTTCATTC |
| | | AGCTCCGGTTCCCAACGATCAAGGCGAGTTACAT |
| | | GATCCCCCATGTTGTGCAAAAAAGCGGTTAGCTC |
| | | CTTCGGTCCTCCGATCGTTGTCAGAAGTAAGTTG |
| | | GCCGCAGTGTTATCACTCATGGTTATGGCAGCAC |
| | | TGCATAATTCTCTTACTGTCATGCCATCCGTAAG |
| | | ATGCTTTTCTGTGACTGGTGAGTACTCAACCAAG |
| | | TCATTCTGAGAATAGTGTATGCGGCGACCGAGTT |
| | | GCTCTTGCCCGGCGTCAATACGGGATAATACCGC |
| | | GCCACATAGCAGAACTTTAAAAGTGCTCATCATT |
| | | GGAAAACGTTCTTCGGGGCGAAAACTCTCAAGGA |
| | | TCTTACCGCTGTTGAGATCCAGTTCGATGTAACC |
| | | CACTCGTGCACCCAACTGATCTTCAGCATCTTTT |
| | | ACTTTCACCAGCGTTTCTGGGTGAGCAAAAACAG |
| | | GAAGGCAAAATGCCGCAAAAAAGGGAATAAGGGC |
| | | GACACGGAAATGTTGAATACTCATACTCTTCCTT |
| | | TTTCAATATTATTGAAGCATTTATCAGGGTTATT |
| | | GTCTCATGAGCGGATACATATTTGAATGTATTTA |
| | | GAAAAATAAACAAATAGGGGTTCCGCGCACATTT |
| | | CCCCGAAAAGTGCCACCTGACGTCGACGGATCGG |
| | | GAGATCTCCCGATCCCCTATGGTGCACTCTCAGT |
| | | ACAATCTGCTCTGATGCCGCATAGTTAAGCCAGT |
| | | ATCTGCTCCCTGCTTGTGTGTTGGAGGTCGCTGA |
| | | GTAGTGCGCGAGCAAAATTTAAGCTACAACAAGG |
| | | CAAGGCTTGACCGACAATTGCATGAAGAATCTGC |
| | | TTAGGGTTAGGCGTTTTGCGCTGCTTCGCGATGT |
| | | ACGGGCCAGATATACGCGTTGACATTGATTATTG |
| | | ACTAGTTATTAATAGTAATCAATTACGGGGTCAT |
| | | TAGTTCATAGCCCATATATGGAGTTCCGCGTTAC |
| | | ATAACTTACGGTAAATGGCCCGCCTGGCTGACCG |
| | | CCCAACGACCCCCGCCCATTGACGTCAATAATGA |
| | | CGTATGTTCCCATAGTAACGCCAATAGGGACTTT |
| | | CCATTGACGTCAATGGGTGGAGTATTTACGGTAA |
| | | ACTGCCCACTTGGCAGTACATCAAGTGTATCATA |
| | | TGCCAAGTACGCCCCCTATTGACGTCAATGACGG |
| | | TAAATGGCCCGCCTGGCATTATGCCCAGTACATG |
| | | ACCTTATGGGACTTTCCTACTTGGCAGTACATCT |
| | | ACGTATTAGTCATCGCTATTACCATGGTGATGCG |
| | | GTTTTGGCAGTACATCAATGGGCGTGGATAGCGG |
| | | TTTGACTCACGGGGATTTCCAAGTCTCCACCCCA |
| | | TTGACGTCAATGGGAGTTTGTTTTGGCACCAAAA |
| | | TCAACGGGACTTTCCAAAATGTCGTAACAACTCC |
| | | GCCCCATTGACGCAAATGGGCGGTAGGCGTGTAC |
| | | GGTGGGAGGTCTATATAAGCAGCGCGTTTTGCCT |
| | | GTACTGGGTCTCTCTGGTTAGACCAGATCTGAGC |
| | | CTGGGAGCTCTCTGGCTAACTAGGGAACCCACTG |
| | | CTTAAGCCTCAATAAAGCTTGCCTTGAGTGCTTC |
| | | AAGTAGTGTGTGCCCGTCTGTTGTGTGACTCTGG |
| | | TAACTAGAGATCCCTCAGACCCTTTTAGTCAGTG |
| | | TGGAAAATCTCTAGCAGTGGCGCCCGAACAGGGA |
| | | CTTGAAAGCGAAAGGGAAACCAGAGGAGCTCTCT |
| | | CGACGCAGGACTCGGCTTGCTGAAGCGCGCACGG |
| | | CAAGAGGCGAGGGGCGGCGACTGGTGAGTACGCC |
| | | AAAAATTTTGACTAGCGGAGGCTAGAAGGAGAGA |
| | | GATGGGTGCGAGAGCGTCAGTATTAAGCGGGGGA |
| | | GAATTAGATCGCGATGGGAAAAAATTCGGTTAAG |
| | | GCCAGGGGGAAAGAAAAAATATAAATTAAAACAT |
| | | ATAGTATGGGCAAGCAGGGAGCTAGAACGATTCG |
| | | CAGTTAATCCTGGCCTGTTAGAAACATCAGAAGG |
| | | CTGTAGACAAATACTGGGACAGCTACAACCATCC |
| | | CTTCAGACAGGATCAGAAGAACTTAGATCATTAT |
| | | ATAATACAGTAGCAACCCTCTATTGTGTGCATCA |
| | | AAGGATAGAGATAAAAGACACCAAGGAAGCTTTA |
| | | GACAAGATAGAGGAAGAGCAAAACAAAAGTAAGA |
| | | CCACCGCACAGCAAGCGGCCGCTGATCTTCAGAC |
| | | CTGGAGGAGGAGATATGAGGGACAATTGGAGAAG |
| | | TGAATTATATAAATATAAAGTAGTAAAAATTGAA |
| | | CCATTAGGAGTAGCACCCACCAAGGCAAAGAGAA |
| | | GAGTGGTGCAGAGAGAAAAAAGAGCAGTGGGAAT |
| | | AGGAGCTTTGTTCCTTGGGTTCTTGGGAGCAGCA |
| | | GGAAGCACTATGGGCGCAGCGTCAATGACGCTGA |
| | | CGGTACAGGCCAGACAATTATTGTCTGGTATAGT |
| | | GCAGCAGCAGAACAATTTGCTGAGGGCTATTGAG |
| | | GCGCAACAGCATCTGTTGCAACTCACAGTCTGGG |
| | | GCATCAAGCAGCTCCAGGCAAGAATCCTGGCTGT |
| | | GGAAAGATACCTAAAGGATCAACAGCTCCTGGGG |
| | | A |
| 48 | DNA Sequence DR628 | ATGGCTCTGCCGGTTACAGCCCTGCTGCTTCCTC |
| | (codon optimized | TCGCCCTCCTTCTTCATGCAGCGCGACCGGATGT |
| | | CGTGATGACCCAGAGCCCCCTGTCTCTGCCCGTC |
| | | ACTCCAGGTGAACCGGCATCAATATCATGTCGAA |
| | | GTAGTCAATCACTCGTTCATTCAAACGCAAATAC |
| | | CTATCTCCACTGGTACCTGCAGAAGCCCGGACAA |
| | | TCACCTCAGTTGCTGATCTACAAGGTATCTAACC |
| | | GCTTCTCAGGCGTTCCAGATAGATTCTCTGGGTC |
| | | TGGTTCCGGGACAGACTTTACGCTCAAAATTAGC |
| | | CGAGTTGAGGCTGAGGACGTAGGGGTGTATTATT |
| | | GTTCCCAAAATACTCATGTTCCGCCCACCTTCGG |
| | | GCAGGGCACCAAACTTGAAATAAAGCGCGGCAGC |
| | | ACTTCAGGAAGCGGCAAACCTGGCTCTGGTGAGG |
| | | GAAGCACGAAGGGCCAGGTACAACTCGTACAATC |
| | | AGGAGCTGAAGTTAAAAAGCCCGGGGCGTCAGTC |
| | | AAAGTGTCCTGCAAGGCCAGTGGCTATACGTTTA |
| | | CTGACTATGAGATGCATTGGGTGCGCCAGGCTCC |
| | | GGGTCAAGGGCTCGAGTGGATGGGAGCACTTGAT |
| | | CCCAAGACGGGTGACACTGCTTACTCTCAGAAAT |
| | | TTAAAGGGCGGGTTACACTCACGGCGGACGAGTC |
| | | CACATCAACGGCGTATATGGAGTTGAGTTCACTG |
| | | CGAAGCGAAGATACGGCTGTCTACTATTGCACCC |
| | | GATTTTATTCATACACGTATTGGGGTCAAGGTAC |
| | | CCTCGTGACTGTTTCATCAACTACAACGCCCGCT |
| | | CCGCGGCCCCCAACTCCTGCTCCCACAATCGCCA |
| | | GTCAACCTCTTAGCTTGCGCCCCGAAGCATGTAG |
| | | ACCCGCGGCCGGTGGGGCTGTGCATACACGCGGA |
| | | CTTGACTTCGCCTGCGACATTTACATTTGGGCTC |
| | | CCTTGGCTGGTACTTGTGGGGTTCTGCTTCTTTC |
| | | TCTTGTTATTACTAAACGCGGCCGGAAAAAGTTG |
| | | TTGTATATCTTTAAGCAACCTTTCATGAGGCCGG |
| | | TACAGACAACGCAAGAGGAGGATGGATGCAGTTG |
| | | TAGATTCCCGGAAGAAGAAGAGGGCGGGTGTGAG |
| | | CTTAGGGTCAAGTTCTCACGATCAGCAGACGCCC |
| | | CGGCCTACCAACAAGGCCAAAATCAACTGTATAA |
| | | TGAACTGAACCTTGGGCGGCGAGAGGAGTATGAC |
| | | GTTCTCGATAAACGCAGAGGGCGCGACCCAGAGA |
| | | TGGGTGGGAAACCGCGGCGCAAAAACCCCCAGGA |
| | | AGGACTCTATAATGAGCTCCAAAAGGATAAAATG |
| | | GCTGAAGCTTACAGTGAAATCGGAATGAAGGGTG |
| | | AACGGAGGCGAGGGAAGGGACACGATGGCTTGTA |
| | | TCAGGGACTTTCCACAGCTACAAAGGATACTTAC |
| | | GACGCCTTGCACATGCAGGCGCTTCCTCCTCGGG |
| | | GGAGTGGAGAGGGCCGCGGATCACTTCTCACATG |
| | | CGGCGACGTAGAGGAAAATCCTGGCCCCATGGCA |
| | | GCACCGGCGCTTTCATGGCGGCTGCCGCTGTTGA |
| | | TACTTCTTTTGCCTCTGGCAACATCATGGGCTTC |
| | | CGCCGCCGTAAATGGGACCTCCCAATTTACTTGC |
| | | TTCTACAACAGTCGCGCGAACATCTCCTGCGTCT |
| | | GGAGCCAGGACGGCGCTCTCCAAGACACGAGCTG |
| | | TCAAGTACATGCCTGGCCGGATAGACGCAGATGG |
| | | AATCAGACCTGCGAGTTGCTCCCTGTATCCCAAG |
| | | CTTCATGGGCTTGCAATCTCATCTTGGGCGCCCC |
| | | AGATAGCCAAAAATTGACTACGGTAGATATTGTC |
| | | ACTTTGAGAGTCCTCTGCCGCGAAGGCGTCAGAT |
| | | GGAGAGTAATGGCAATTCAAGATTTTAAACCATT |
| | | TGAGAATCTGCGACTTATGGCCCCTATCAGTTTG |
| | | CAAGTAGTACATGTGGAAACGCATCGATGCAATA |
| | | TATCATGGGAGATAAGCCAGGCGAGTGATTTCTT |
| | | CGAAAGGCACCTCGAGTTTGAGGCCCGAACTTTG |
| | | AGCCCAGGTCACACTTGGGAAGAAGCACCCTTGC |
| | | TCACTCTTAAACAGAAACAGGAGTGGATATGTTT |
| | | GGAGACACTTACTCCGGACACGCAGTATGAGTTC |
| | | CAAGTTAGAGTGAAACCCCTGCAAGGAGAATTCA |
| | | CCACTTGGTCTCCATGGAGTCAACCATTGGCTTT |
| | | TCGCACTAAACCCGCGGCGCTTGGAAAGGACACT |
| | | ATACCGTGGCTTGGACATTTGCTCGTCGGTCTGA |
| | | GCGGAGCTTTCGGGTTTATTATATTGGTCTATTT |
| | | GCTCATTAATTGCAGGAATACAGGCCCGTGGCTG |
| | | AAGAAGGTTCTCAAATGTAATACACCCGACCCAT |
| | | CTAAGTTTTTCTCTCAACTCTCCTCTGAGCACGG |
| | | AGGAGACGTCCAAAAGTGGCTTTCAAGCCCCTTC |
| | | CCGAGTAGTTCCTTTAGTCCGGGTGGACTCGCGC |
| | | CGGAGATTAGTCCCCTGGAAGTATTGGAACGAGA |
| | | CAAGGTTACTCAGCTCCTTCTGCAGCAAGACAAG |
| | | GTTCCTGAACCCGCTTCCTTGTCCAGTAATCATT |
| | | CACTGACATCTTGCTTTACCAACCAAGGATATTT |
| | | TTTTTTCCATCTTCCTGACGCCCTGGAAATTGAG |
| | | GCTTGCCAAGTGTACTTTACGTACGACCCTTATT |
| | | CCGAAGAAGACCCAGACGAGGGCGTAGCGGGTGC |
| | | ACCAACTGGGTCCAGCCCTCAACCGCTTCAGCCG |
| | | CTTAGTGGGGAAGACGATGCATATTGTACTTTCC |
| | | CCAGTAGGGACGATCTTCTTCTCTTTTCCCCAAG |
| | | TCTGCTCGGGGGCCCGAGCCCTCCATCAACCGCC |
| | | CCAGGCGGTTCAGGCGCCGGCGAAGAGAGAATGC |
| | | CTCCAAGCCTTCAAGAAAGAGTTCCACGGGACTG |
| | | GGATCCTCAGCCACTTGGTCCACCGACTCCCGGT |
| | | GTTCCTGATCTGGTTGACTTCCAGCCTCCACCAG |
| | | AGCTTGTGCTTAGGGAGGCAGGCGAAGAAGTGCC |
| | | CGACGCTGGGCCAAGGGAGGGAGTTAGTTTCCCG |
| | | TGGTCTAGACCGCCTGGCCAGGGCGAGTTCAGAG |
| | | CTTTGAACGCGCGGCTCCCTTTGAATACCGACGC |
| | | TTACTTGTCATTGCAAGAGCTGCAAGGGCAAGAC |
| | | CCGACACACTTGGTGTGATAA |
| 49 | G4Sx 3 scFv Linker | GGGGSGGGGSGGGGS |
| 50 | albumin-binding peptide | DICLPRWGCLW |

What is claimed is:

1. A GPC3 CAR comprising an antigen binding domain that selectively binds to the human glypican-3 beta subunit (SEQ ID NO:2) wherein the antigen binding domain is an scFv having at least 90% %, 95%, 99%, or 100% sequence identity to SEQ ID NO:10 or SEQ ID NO: 11.

2. The GPC3 CAR of claim 1 wherein the GPC3 CAR comprises a CD3 zeta signaling domain and a costimulatory domain selected from the costimulatory domains of C28 or 4-1BB.

3. The GPC3 CAR of claim 1 wherein the GPC3 CAR has at least 90%, 95%, 99%, or 100% sequence identity to a GPC3 CAR selected from the group consisting of SEQ ID NO: 34, SEQ ID NO: 35, SEQ ID NO: 36, SEQ ID NO: 37, SEQ ID NO: 38, SEQ ID NO: 39, and SEQ ID NO: 40.

4. The GPC3 CAR of claim 1 wherein the GPC3 CAR is selected from the group consisting of SEQ ID NO: 34, SEQ ID NO: 35, SEQ ID NO: 36, SEQ ID NO: 37, SEQ ID NO: 38, SEQ ID NO: 39, and SEQ ID NO: 40.

5. A nucleic acid sequence encoding the GPC3 CAR of any one of claims 1-4.

6. A recombinant vector comprising the nucleic acid sequence of claim 5.

7. The recombinant vector of claim 6 wherein the recombinant vector is a lentiviral vector or a retroviral vector.

8. The recombinant vector of claim 6 or 7 wherein the vector further comprises a nucleic acid sequence encoding an ortho CD122 receptor.

9. The recombinant viral vector of claim 8 wherein the nucleic acid sequence encoding the GPC3 CAR and the and the nucleic acid sequence encoding the ortho CD122 receptor are operably linked to at least one expression control sequence functional in a mammalian T cell.

10. The recombinant viral vector of claim 9 wherein nucleic acid sequence encoding an orthogonal receptor and the nucleic acid sequence encoding the GPC3 CAR the nucleic acid sequence encoding an ortho CD122 receptor are operably linked to single expression control sequence and the wherein the nucleic acid sequences encoding the GPC3 CAR and the ortho CD122 receptor are separated by an IRES or T2A sequence.

11. A recombinantly modified T cell comprising a recombinant viral vector of claim 10.

12. An ortho GPC3 CAR T cell, the cell expressing an ortho CD122 and a GPC3 CAR wherein the ortho CD122 comprises amino acid substitutions at positions 133 and/or 134 numbered in accordance with wild-type hCD122.

13. The ortho GPC3 CAR T cell of claim 12 wherein the GPC3 CAR comprises the amino acid sequence of SEQ ID NO: 37; and (b) the ortho CD122 having the amino acid sequence of SEQ ID NO:4.

14. A method of making an ortho GPC3 CAR T cell, the method comprising the steps of:
a) obtaining a sample of human peripheral blood mononuclear cells (PBMCs);
b) contacting the sample of PBMCs by with magnetic beads coated with CD3 and CD28 ligands to provide a population of activated PBMCs
c) isolating from the an activated PBMCs cell population CD8+ and CD4+ T cells;
d) contacting the isolated population CD8+ and CD4+ T cells with a recombinant lentiviral vector, the lentiviral vector comprising an expression cassette consisting of a nucleic 9 acid sequence encoding from 5′ to 3′:
a promoter active in a T cell e.g. the EF1a promoter;
a nucleic acid sequence encoding a signal peptide (
a nucleic acid sequence encoding a GPC3 CAR;
a T2A sequence (SEQ ID NO:41)
a nucleic acid sequence encoding a signal peptide
a nucleic acid sequence encoding the ortho CD122 of SEQ ID NO:4
such that a fraction of the isolated population CD8+ and CD4+ T cells is transduced with the lentiviral vector;
e) contacting the population of cells obtained from step (d) with an ortho IL2, the ortho IL2 selected from the group consisting of a human IL2 mutein containing the of amino acid substitutions E15S/H16Q/L19V/D20L/Q22K/M23A; an ortho IL2 of SEQ ID NO 9 (STK-007), or pegylated variant thereof (e.g., STK-009).

15. A ortho GPC3 CAR T cell prepared in accordance with claim 14.

16. A method of treating or preventing a neoplastic disease, disorder, or condition in a mammalian subject in need of treatment or prevention, the method comprising administering to said subject a therapeutically effective amount of the ortho GPC3 CAR T cell of claim 15 in combination with a therapeutically effective dose of ortho IL.

17. The method of claim 14 wherein after step (a) but prior to step (b), the population of cells is enriched for CD8+ or CD4+ T cells.

18. The method of claim 14 wherein ortho IL2 employed ex vivo in step (b) is different than the orthogonal ligand used in vivo in step (c).

19. The method of claim 14 wherein ortho wherein prior to step (d) the subject is treated with a lymphodepleting regimen.

20. A method of treating relapse following ortho GPC3 CAR therapy in a subject, the method comprising administering to the subject a therapeutically effective amount of an ortho IL2 such that the orthogonal ligand induces the activation and/or proliferation of the ortho GPC3 CAR T cell in the subject.

21. The method of claim 20 wherein the ortho IL2 is STK-009.

22. A method of generating an ortho GPC3 CAR T cell product substantially enriched for a population of ortho GPC3 CAR T cells, the method comprising the steps of:
(a) Isolating a population of cells from a mammalian subject, the population of cells comprising T cells;
(b) Contacting the isolated population of T cells from step (a) a recombinant vector comprising a first nucleic acid sequence encoding a GPC3 CAR and a second nucleic encoding an ortho CD122, the first and second nucleic acid sequences operably linked to an expression control sequence operable in a mammalian T cell, wherein the first and second nucleic acid sequences are separated by an IRES or T2A nucleic acid sequence, such that the cell expresses the GPC3 CAR and the ortho CD122 (ortho GPC3 CAR T cell);
(c) Contacting the isolated population of cells from step (b) ex vivo with a quantity of a ortho IL2 sufficient to induce proliferation of cells transduced by the contacting of step (b),
wherein the contacting with the ortho IL2 is maintained for a period of time period of time such that the fraction of cells expressing the GPC3 CAR and the ortho CD122 comprise at least 10% of the population.


SEQUENCE LISTING (50 sequences)

SEQ ID NO: 1 - protein, 580 aa, Homo sapiens
  REGION 1..580: note = Description of sequence: Human GPC3 precursor (580 amino acids signal peptide underlined; UniProt Ref: P51654
  DOMAIN 1..24: note = signal peptide
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 2 - protein, 196 aa, Homo sapiens
  REGION 1..196: note = Description of sequence: Human Glypican-3 beta subunit UniProt Ref: P51654 AAs 359-554 numbered in accordance with hGPC3 precursor SEQ ID NO:1
SAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHN

SEQ ID NO: 3 - protein, 551 aa, Homo sapiens
  REGION 1..551: note = Description of sequence: Wild Type Human CD122 precursor (including signal peptide underlined) UniProt Ref: P14784
  DOMAIN 1..26: note = signal peptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 4 - protein, 525 aa, Homo sapiens
  REGION 1..525: note = Description of sequence: Mature wild type hCD122 (without signal peptide) UniProt Ref: P14784
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 5 - protein, 551 aa, Homo sapiens
  REGION 1..551: note = Description of sequence: hoCD122 precursor with signal peptide (underlined) containing amino acid substitutions H133D and Y134F (bold numbered in accordance with mature wt CD122 protein SEQ ID NO:4)
  DOMAIN 1..25: note = signal peptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 6 - protein, 525 aa, Homo sapiens
  REGION 1..525: note = Description of sequence: Mature hoCD122 without signal peptide containing amino acid substitutions H133D and Y134F (bold numbered in accordance with mature wt CD122 protein SEQ ID NO:4)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 7 - protein, 153 aa, Homo sapiens
  REGION 1..153: note = Description of sequence: Wild Type Human IL2 Precursor (signal peptide underlined) UniProt P60568)
  DOMAIN 1..20: note = signal peptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 133 aa, Homo sapiens [sequence omitted]

SEQ ID NO: 9 - protein, 132 aa, Homo sapiens
  REGION 1..132: note = Description of sequence: hIL2 mutein having elation of Ala1 and AA subsitutions E15S/H16Q/L19V/D20L/Q22K/M23A (STK-007)
PTSSSTKKTQLQLSQLLVLLKAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 243 aa, Homo sapiens
  REGION 1..243: note = Description of sequence: GC33 scFv with G4Sx3 linker (underlined)
  DOMAIN 114..128: note = linker
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 11 - protein, 246 aa, Homo sapiens
  REGION 1..246: note = Description of sequence: GC33 scFv with Whitlow linker (underlined)
  DOMAIN 114..131: note = linker
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGSTSGSGKPGSGEGSTKGQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 12 - protein, 5 aa, Homo sapiens
  REGION 1..5: note = Description of sequence: GC33 Heavy Chain CDR1
DYEMH

SEQ ID NO: 13 - protein, 17 aa, Homo sapiens
  REGION 1..17: note = Description of sequence: GC33 Heavy Chain CDR2
ALDPKTGDTAYSQKFKG

SEQ ID NO: 14 - protein, 6 aa, Homo sapiens
  REGION 1..6: note = Description of sequence: GC33 Heavy Chain CDR3
FYSYTY

SEQ ID NO: 15 - protein, 16 aa, Homo sapiens
  REGION 1..16: note = Description of sequence: GC33 Light Chain CDR1
RSSQSLVHSNANTYLH

SEQ ID NO: 16 - protein, 7 aa, Homo sapiens
  REGION 1..7: note = Description of sequence: GC33 Light Chain CDR2
KVSNRFS

SEQ ID NO: 17 - protein, 9 aa, Homo sapiens
  REGION 1..9: note = Description of sequence: GC33 Light Chain CDR3
SQNTHVPPT

SEQ ID NO: 18 - protein, 18 aa, Homo sapiens
  REGION 1..18: note = Description of sequence: Whitlow Linker
GSTSGSGKPGSGEGSTKG

SEQ ID NO: 19 - protein, 235 aa, Homo sapiens
  REGION 1..235: note = Description of sequence: hCD8A precursor including signal peptide (underlined) UniProt Ref: P01732
  DOMAIN 1..21: note = signal peptide
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 20 - protein, 21 aa, Homo sapiens
  REGION 1..21: note = Description of sequence: Human CD8a signal peptide (AAs 1-21 of hCD8a precursor numbered in accordance with hCD8A precursor including signal peptide UniProt Ref: P01732)
MALPVTALLLPLALLLHAARP

SEQ ID NO: 21 - protein, 62 aa, Homo sapiens
  REGION 1..62: note = Description of sequence: Human CD8a hinge 117 to 178
ALSNSIMYFSHFVPVFLPAKPTTTPAPRPPTPAPTIASQPLSLRPEACRPAAGGAVHTRGLD

SEQ ID NO: 22 - protein, 41 aa, Homo sapiens
  REGION 1..41: note = Description of sequence: Human CD8a hinge fragment
TTTPAPRPPTPAPTIASQPLSLRPEACRPAAGGAVHTRGLD

SEQ ID NO: 23 - protein, 62 aa, Homo sapiens
  REGION 1..62: note = Description of sequence: Human CD8a hinge region with modification of unpaired CYS to SER substitution at 117 to 178; Schonfeld et al European patent application 3115373A1
ALSNSIMYFSHFVPVFLPAKPTTTPAPRPPTPAPTIASQPLSLRPEASRPAAGGAVHTRGLD

SEQ ID NO: 24 - protein, 28 aa, Homo sapiens
  REGION 1..28: note = Description of sequence: Human CD8a transmembrane domain 1
FACDIYIWAPLAGTCGVLLLSLVITLYC

SEQ ID NO: 25 - protein, 25 aa, Homo sapiens
  REGION 1..25: note = Description of sequence: Human CD8a transmembrane domain 2
FACDIYIWAPLAGTCGVLLLSLVIT

SEQ ID NO: 26 - protein, 69 aa, Homo sapiens
  REGION 1..69: note = Description of sequence: Partial human CD8a hinge domain CD8a transmembrane domain (AAs 138-206 of hCD8a precursor numbered in accordance with hCD8A precursor including signal peptide UniProt Ref: P01732)
TTTPAPRPPTPAPTIASQPLSLRPEACRPAAGGAVHTRGLDFACDIYIWAPLAGTCGVLLLSLVITLYC

SEQ ID NO: 27 - protein, 66 aa, Homo sapiens
  REGION 1..66: note = Description of sequence: Human CD8a hinge and transmembrane domain (AAs 138-203 of hCD8a precursor numbered in accordance with hCD8A precursor including signal peptide UniProt Ref: P01732)
TTTPAPRPPTPAPTIASQPLSLRPEACRPAAGGAVHTRGLDFACDIYIWAPLAGTCGVLLLSLVIT

SEQ ID NO: 28 - protein, 107 aa, Homo sapiens
  REGION 1..107: note = Description of sequence: Human CD28 ECD fragment transmembrane and Co-stimulatory domain (UniProt P10747 AAs114-220 numbered in accordance with precursor including signal peptide)
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 29 - protein, 40 aa, Homo sapiens
  REGION 1..40: note = Description of sequence: Human CD28 ECD fragment
IEVMYPPPYLDNEKSNGTIIHVKGKHLCPSPLFPGPSKPF

SEQ ID NO: 30 - protein, 25 aa, Homo sapiens
  REGION 1..25: note = Description of sequence: Human CD28 transmembrane domain
WVLVVVGGVLACYSLLVTVAFIIFW

SEQ ID NO: 31 - protein, 42 aa, Homo sapiens
  REGION 1..42: note = Description of sequence: Human CD28 co-stimulatory domain
VRSKRSRLLHSDYMNMTPRRPGPTRKHYQPYAPPRDFAAYRS

SEQ ID NO: 32 - protein, 42 aa, Homo sapiens
  REGION 1..42: note = Description of sequence: Human 4-1BB (CD137TNFRSF9) co-stimulatory domain (UniProt Q07011 AAs 129-225 numbered in accordance with precursor including signal peptide)
KRGRKKLLYIFKQPFMRPVQTTQEEDGCSCRFPEEEEGGCEL

SEQ ID NO: 33 - protein, 112 aa, Homo sapiens
  REGION 1..112: note = Description of sequence: Human CD3 zeta domain (UniProt P20963 AAs 52-164 numbered in accordance with CD3z precursor including signal peptide)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 486 aa, Homo sapiens
  REGION 1..486: note = Description of sequence: hCD8a signal peptide (italics); Whitlow Linker underlined); hCD28 transmembrane and co-stimulatory domain; hCD3zeta domain
  DOMAIN 1..21: note = signal peptide
  DOMAIN 135..152: note = linker
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGSTSGSGKPGSGEGSTKGQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 483 aa, Homo sapiens
  REGION 1..483: note = Description of sequence: hCD8a signal peptide; G4Sx3 Linker; hCD28 transmembrane and co-stimulatory domain; hCD3-zeta domain
  DOMAIN 1..21: note = signal peptide
  DOMAIN 135..149: note = linker
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 490 aa, Homo sapiens
  REGION 1..490: note = Description of sequence: CD8a signal peptide GPC3 scFv whitlow linker underlined hCD8a hinge and transmembrane h4-1BB co-stimulatory domain hCD3-zeta domain
  DOMAIN 1..21: note = signal peptide
  DOMAIN 135..152: note = linker
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGSTSGSGKPGSGEGSTKGQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 1061 aa, Homo sapiens
  REGION 1..1061: note = Description of sequence: DR625 GPC3 CAR
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGSTSGSGKPGSGEGSTKGQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 38 - protein, 1061 aa, Homo sapiens
  REGION 1..1061: note = Description of sequence: DR626 GPC3 CAR
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGEGRGSLLTCGDVEENPGPMALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGSTSGSGKPGSGEGSTKGQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 488 aa, Homo sapiens
  REGION 1..488: note = Description of sequence: DR628 GPC3 CAR
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGSTSGSGKPGSGEGSTKGQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 1062 aa, Homo sapiens
  REGION 1..1062: note = Description of sequence: DR1581 CAR (DR625 with CD28 replaced with CD8a TM 41bb)
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGSTSGSGKPGSGEGSTKGQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 41 - DNA, 63 bp, synthetic construct
  source 1..63: mol_type = other DNA
  misc_feature 1..63: note = Description of sequence:synthetic construct
gggagtggagagggccgcggatcacttctcacatgcggcgacgtagaggaaaatcctggcccc

SEQ ID NO: 42 - DNA, 3183 bp, synthetic construct
  source 1..3183: mol_type = other DNA
  misc_feature 1..3183: note = Description of sequence:synthetic construct
atggctctgcctgtgacagctctgctgctgcctctggctctgcttctgcatgccgccagacctgacgtggtcatgacacagagccctctgagcctgcctgtgacacctggcgaacctgccagcatcagctgtagaagcagccagagcctggtgcacagcaacgccaacacatacctgcactggtatctgcagaagcccggccagtctcctcagctgctgatctacaaggtgtccaacagattcagcggcgtgcccgacagattctctggctctggatctggcaccgacttcaccctgaagatctccagagtggaagccgaggacgtgggcgtgtactactgcagccagaacacacacgtgccacctacattcggccagggcaccaagctggaaatcaagaggggatccacaagcggctctggcaagcctggaagcggagagggatctaccaaaggccaggtgcagctggttcagtctggcgccgaagtgaagaaacctggcgcctctgtgaaggtgtcctgcaaggcaagcggctacacattcaccgactacgagatgcactgggtccgacaggctccaggacagggacttgaatggatgggagccctagatcctaagaccggcgatacagcctacagccagaaattcaagggaagagtgaccctgaccgccgacgagagcacatctaccgcctacatggaactgagcagcctgagaagcgaggacaccgccgtgtattactgtaccagattctactcctacacctactggggccagggaaccctggtcacagtctcttctgccgccgctatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgatccaagcggagccggctgctgcacagcgactacatgaacatgacccctagacggcccggaccaaccagaaagcactaccagccttacgctcctcctcgggatttcgccgcctacagatccagagtgaagttctccagaagcgccgacgctcctgcctatcagcagggacagaatcagctgtacaacgagctgaacctggggcgcagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgaacgcagaagaggcaagggccacgatggactgtaccagggactgagcaccgccaccaaggatacctatgacgccctgcacatgcaggctctgccacctagagggtccggagaaggcagaggatctctgctgacatgcggcgacgtggaagagaaccctgggcccatggctgcccctgctctgtcttggagactgcctctgctgatcctgctgctgcctctggctacatcttgggcctctgccgccgtgaatggcaccagccagttcacctgtttctacaacagccgggccaacatcagctgcgtgtggtcacaagatggcgccctgcaggataccagctgtcaggttcacgcctggcctgatcggagaagatggaaccagacatgcgagctgctgcccgtgtctcaggcttcctgggcctgtaatctgatcctgggagcccctgacagccagaaactgaccaccgtggatatcgtgaccctgcgggtgctgtgtagagaaggcgttcgttggagagtgatggccattcaggacttcaagcccttcgagaacctgcggctgatggccccaattagcctgcaggttgtgcacgtggaaacccacagatgcaatatcagctgggagatcagccaggccagcgacttcttcgagcggcacctggaatttgaggcccggacactgtctccaggccacacctgggaagaagcccctctgctcaccctgaagcagaaacaagagtggatctgcctggaaaccctgacacctgacacacagtacgagttccaagtgcgcgtgaagcctctccagggcgagttcacgacgtggtccccttggagtcagcccctggcctttagaacaaagcctgccgctctgggcaaagacacaatcccttggctgggccatctgctcgttggactgtctggcgccttcggcttcatcatcctggtgtacctgctgatcaactgccggaacaccggaccttggctgaagaaagtgctgaagtgcaacacccctgatccgagcaagttctttagccagctgagcagcgagcacggcggagatgttcagaagtggctgtctagcccatttcctagcagcagcttcagccctggtggactggcccctgagattagccctctggaagtgctggaacgggacaaagtgacccagctgcttctgcagcaggacaaggtgccagaacctgccagcctgtccagcaatcacagcctgaccagctgttttacaaaccagggctacttcttcttccatctgcctgacgcactggaaatcgaggcctgccaggtgtacttcacctacgatccctacagcgaagaggaccccgatgaaggtgttgccggtgctcctaccggaagctctcctcaacctctgcaaccactgagcggcgaggatgacgcctactgcacattccccagcagggacgatctgctgctgttcagcccatctctgctcggcggaccttctccaccatctacagctccaggtggaagcggagccggcgaggaaagaatgccacctagcctgcaagagcgggtgcccagagattgggaccctcaaccacttggccctccaacacctggcgtgccagacctggttgatttccagcctcctccagagctggtgctgagagaagctggcgaagaagtgccagacgccggacctagagagggcgttagctttccttggagcagacctcctggacaaggcgagttcagagccctgaatgccagactgcccctgaacaccgatgcctacctgtctctgcaagaactgcagggacaggatcccacacacctggtc

SEQ ID NO: 43 - DNA, 3183 bp, synthetic construct
  source 1..3183: mol_type = other DNA
  misc_feature 1..3183: note = Description of sequence:synthetic construct
atggctgcccctgctctgtcttggagactgcctctgctgatcctgctgctgcctctggctacatcttgggcctctgccgccgtgaatggcaccagccagttcacctgtttctacaacagccgggccaacatcagctgcgtgtggtcacaagatggcgccctgcaggataccagctgtcaggttcacgcctggcctgatcggagaagatggaaccagacatgcgagctgctgcccgtgtctcaggcttcctgggcctgtaatctgatcctgggagcccctgacagccagaaactgaccaccgtggatatcgtgaccctgcgggtgctgtgtagagaaggcgttcgttggagagtgatggccattcaggacttcaagcccttcgagaacctgcggctgatggccccaattagcctgcaggttgtgcacgtggaaacccacagatgcaatatcagctgggagatcagccaggccagcgacttcttcgagcggcacctggaatttgaggcccggacactgtctccaggccacacctgggaagaagcccctctgctcaccctgaagcagaaacaagagtggatctgcctggaaaccctgacacctgacacacagtacgagttccaagtgcgcgtgaagcctctccagggcgagttcacgacgtggtccccttggagtcagcccctggcctttagaacaaagcctgccgctctgggcaaagacacaatcccttggctgggccatctgctcgttggactgtctggcgccttcggcttcatcatcctggtgtacctgctgatcaactgccggaacaccggaccttggctgaagaaagtgctgaagtgcaacacccctgatccgagcaagttctttagccagctgagcagcgagcacggcggagatgttcagaagtggctgtctagcccatttcctagcagcagcttcagccctggtggactggcccctgagattagccctctggaagtgctggaacgggacaaagtgacccagctgcttctgcagcaggacaaggtgccagaacctgccagcctgtccagcaatcacagcctgaccagctgttttacaaaccagggctacttcttcttccatctgcctgacgcactggaaatcgaggcctgccaggtgtacttcacctacgatccctacagcgaagaggaccccgatgaaggtgttgccggtgctcctaccggaagctctcctcaacctctgcaaccactgagcggcgaggatgacgcctactgcacattccccagcagggacgatctgctgctgttcagcccatctctgctcggcggaccttctccaccatctacagctccaggtggaagcggagccggcgaggaaagaatgccacctagcctgcaagagcgggtgcccagagattgggaccctcaaccacttggccctccaacacctggcgtgccagacctggttgatttccagcctcctccagagctggtgctgagagaagctggcgaagaagtgccagacgccggacctagagagggcgttagctttccttggagcagacctcctggacaaggcgagttcagagccctgaatgccagactgcccctgaacaccgatgcctacctgtctctgcaagaactgcagggacaggatcccacacacctggtcgggagcggagaaggcagaggaagcttgctgacatgcggcgacgtggaagagaaccctggtcccatggctctgcctgtgacagctctgctgctgcctctggctctgcttctgcatgccgcccgtccggacgtggtcatgacacagagccctctgagcctgcctgtgacacctggcgaacctgccagcatcagctgtagaagcagccagagcctggtgcacagcaacgccaacacatacctgcactggtatctgcagaagcccggccagtctcctcagctgctgatctacaaggtgtccaacagattcagcggcgtgcccgacagattctctggctctggatctggcaccgacttcaccctgaagatctccagagtggaagccgaggacgtgggcgtgtactactgcagccagaacacacacgtgccacctacattcggccagggcaccaagctggaaatcaagaggggatccacaagcggctctggcaagcctggaagcggagagggatctaccaaaggccaggtgcagctggttcagtctggcgccgaagtgaagaaacctggcgcctctgtgaaggtgtcctgcaaggcaagcggctacacattcaccgactacgagatgcactgggtccgacaggctccaggacagggacttgaatggatgggagccctagatcctaagaccggcgatacagcctacagccagaaattcaagggaagagtgaccctgaccgccgacgagagcacatctaccgcctacatggaactgagcagcctgagaagcgaggacaccgccgtgtattactgtaccagattctactcctacacctactggggccagggaaccctggtcacagtctcttctgcggccgctatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgatccaagcggagccggctgctgcacagcgactacatgaacatgacccctagacggcccggaccaaccagaaagcactaccagccttacgctcctcctcgggatttcgccgcctacagatccagagtgaagttctccagaagcgccgacgctcctgcctatcagcagggacagaatcagctgtacaacgagctgaacctggggcgcagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgaacgcagaagaggcaagggccacgatggactgtaccagggactgagcaccgccaccaaggatacctatgacgccctgcacatgcaggctctgccacctaga

SEQ ID NO: 44 - DNA, 3186 bp, synthetic construct
  source 1..3186: mol_type = other DNA
  misc_feature 1..3186: note = Description of sequence:synthetic construct
atggctctgcctgtgacagctctgctgctgcctctggctctgcttctgcatgccgccagacctgacgtggtcatgacacagagccctctgagcctgcctgtgacacctggcgaacctgccagcatcagctgtagaagcagccagagcctggtgcacagcaacgccaacacatacctgcactggtatctgcagaagcccggccagtctcctcagctgctgatctacaaggtgtccaacagattcagcggcgtgcccgacagattctctggctctggatctggcaccgacttcaccctgaagatctccagagtggaagccgaggacgtgggcgtgtactactgcagccagaacacacacgtgccacctacattcggccagggcaccaagctggaaatcaagaggggatccacaagcggctctggcaagcctggaagcggagagggatctaccaaaggccaggtgcagctggttcagtctggcgccgaagtgaagaaacctggcgcctctgtgaaggtgtcctgcaaggcaagcggctacacattcaccgactacgagatgcactgggtccgacaggctccaggacagggacttgaatggatgggagccctagatcctaagaccggcgatacagcctacagccagaaattcaagggaagagtgaccctgaccgccgacgagagcacatctaccgcctacatggaactgagcagcctgagaagcgaggacaccgccgtgtattactgtaccagattctactcctacacctactggggccagggaaccctggtcacagtctcgagcactacgaccccggcacctcgccctcccacccctgcgcctactattgcctcacagccgctgtccctgcgccctgaggcctgtcgcccagctgccggaggggccgtgcacacacgcggactggatttcgcctgcgatatttatatctgggctccgctcgccggaacctgtggtgtgctcttgctgtccctcgtaatcaccctttactgtaagaggggacggaaaaagttgctctacattttcaagcagccatttatgcggcccgttcagacaacccaggaagaggacggttgcagttgcagatttcccgaggaagaggaaggaggttgcgagttgagagtgaagttctccagaagcgccgacgctcctgcctatcagcagggacagaatcagctgtacaacgagctgaacctggggcgcagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgaacgcagaagaggcaagggccacgatggactgtaccagggactgagcaccgccaccaaggatacctatgacgccctgcacatgcaggctctgccacctagagggtccggagaaggcagaggatctctgctgacatgcggcgacgtggaagagaaccctgggcccatggctgcccctgctctgtcttggagactgcctctgctgatcctgctgctgcctctggctacatcttgggcctctgccgccgtgaatggcaccagccagttcacctgtttctacaacagccgggccaacatcagctgcgtgtggtcacaagatggcgccctgcaggataccagctgtcaggttcacgcctggcctgatcggagaagatggaaccagacatgcgagctgctgcccgtgtctcaggcttcctgggcctgtaatctgatcctgggagcccctgacagccagaaactgaccaccgtggatatcgtgaccctgcgggtgctgtgtagagaaggcgttcgttggagagtgatggccattcaggacttcaagcccttcgagaacctgcggctgatggccccaattagcctgcaggttgtgcacgtggaaacccacagatgcaatatcagctgggagatcagccaggccagcgacttcttcgagcggcacctggaatttgaggcccggacactgtctccaggccacacctgggaagaagcccctctgctcaccctgaagcagaaacaagagtggatctgcctggaaaccctgacacctgacacacagtacgagttccaagtgcgcgtgaagcctctccagggcgagttcacgacgtggtccccttggagtcagcccctggcctttagaacaaagcctgccgctctgggcaaagacacaatcccttggctgggccatctgctcgttggactgtctggcgccttcggcttcatcatcctggtgtacctgctgatcaactgccggaacaccggaccttggctgaagaaagtgctgaagtgcaacacccctgatccgagcaagttctttagccagctgagcagcgagcacggcggagatgttcagaagtggctgtctagcccatttcctagcagcagcttcagccctggtggactggcccctgagattagccctctggaagtgctggaacgggacaaagtgacccagctgcttctgcagcaggacaaggtgccagaacctgccagcctgtccagcaatcacagcctgaccagctgttttacaaaccagggctacttcttcttccatctgcctgacgcactggaaatcgaggcctgccaggtgtacttcacctacgatccctacagcgaagaggaccccgatgaaggtgttgccggtgctcctaccggaagctctcctcaacctctgcaaccactgagcggcgaggatgacgcctactgcacattccccagcagggacgatctgctgctgttcagcccatctctgctcggcggaccttctccaccatctacagctccaggtggaagcggagccggcgaggaaagaatgccacctagcctgcaagagcgggtgcccagagattgggaccctcaaccacttggccctccaacacctggcgtgccagacctggttgatttccagcctcctccagagctggtgctgagagaagctggcgaagaagtgccagacgccggacctagagagggcgttagctttccttggagcagacctcctggacaaggcgagttcagagccctgaatgccagactgcccctgaacaccgatgcctacctgtctctgcaagaactgcagggacaggatcccacacacctggtc

SEQ ID NO: 45 - DNA, 1464 bp, synthetic construct
  source 1..1464: mol_type = other DNA
  misc_feature 1..1464: note = Description of sequence:synthetic construct
atggctctgcctgtgacagctctgctgctgcctctggctctgcttctgcatgccgctagacctgacgtggttatgacacagagccctctgagcctgcctgtgacacctggcgaacctgccagcatcagctgtagaagcagccagagcctggtgcacagcaacgccaacacatacctgcactggtatctgcagaagcccggccagtctcctcagctgctgatctacaaggtgtccaacagattcagcggcgtgcccgacagattctctggctctggatctggcaccgacttcaccctgaagatctccagagtggaagccgaggacgtgggcgtgtactactgcagccagaacacacacgtgccacctacattcggccagggcaccaagctggaaatcaagaggggatccacaagcggctctggcaagcctggaagcggagagggatctaccaaaggccaggtgcagctggttcagtctggcgccgaagtgaagaaacctggcgcctctgtgaaggtgtcctgcaaggcaagcggctacacattcaccgactacgagatgcactgggtccgacaggctccaggacagggacttgaatggatgggagccctagatcctaagaccggcgatacagcctacagccagaaattcaagggaagagtgaccctgaccgccgacgagagcacatctaccgcctacatggaactgagcagcctgagaagcgaggacaccgccgtgtattactgtaccagattctactcctacacctactggggccagggaaccctggtcacagtctcttctacaacaacccctgctcctagacctcctacaccagctcctacaatcgccagccagcctctgtctctgaggccagaggcttgtagacctgctgctggcggagccgtgcatacaagaggactggatttcgcctgcgacatctatatctgggcccctctggccggaacatgtggcgttttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgacgctcctgcctatcagcagggacagaatcagctgtacaacgagctgaacctggggcgcagagaagagtacgacgtgctggataagcggagaggcagagatcctgagatgggcggaaagccccagcggagaaagaatcctcaagagggcctgtataatgagctgcagaaagacaagatggccgaggcctacagcgagatcggaatgaagggcgaacgcagaagaggcaagggccacgatggactgtaccagggactgagcaccgccaccaaggatacctatgacgccctgcacatgcaggctctgccacctaga

SEQ ID NO: 46 - DNA, 3189 bp, synthetic construct
  source 1..3189: mol_type = other DNA
  misc_feature 1..3189: note = Description of sequence:synthetic construct
atggctctgccggttacagccctgctgcttcctctcgccctccttcttcatgcagcgcgaccggatgtcgtgatgacccagagccccctgtctctgcccgtcactccaggtgaaccggcatcaatatcatgtcgaagtagtcaatcactcgttcattcaaacgcaaatacctatctccactggtacctgcagaagcccggacaatcacctcagttgctgatctacaaggtatctaaccgcttctcaggcgttccagatagattctctgggtctggttccgggacagactttacgctcaaaattagccgagttgaggctgaggacgtaggggtgtattattgttcccaaaatactcatgttccgcccaccttcgggcagggcaccaaacttgaaataaagcgcggcagcacttcaggaagcggcaaacctggctctggtgagggaagcacgaagggccaggtacaactcgtacaatcaggagctgaagttaaaaagcccggggcgtcagtcaaagtgtcctgcaaggccagtggctatacgtttactgactatgagatgcattgggtgcgccaggctccgggtcaagggctcgagtggatgggagcacttgatcccaagacgggtgacactgcttactctcagaaatttaaagggcgggttacactcacggcggacgagtccacatcaacggcgtatatggagttgagttcactgcgaagcgaagatacggctgtctactattgcacccgattttattcatacacgtattggggtcaaggtaccctcgtgactgtttcatcagctgccgcgatcgaggtgatgtatcctccgccctacttggacaacgaaaagtccaacggcaccatcattcatgtgaaaggcaaacacctctgcccttccccccttttccccggcccaagcaagcccttctgggtcttggtagtagtgggtggggtgctcgcgtgctactctctgctggtcacagtggccttcattatcttttgggtccggtcaaaacgctctcgactgcttcactctgattacatgaatatgactcccagacggccagggcctacgagaaagcattaccaaccttatgcaccacctcgggatttcgcagcatatcgatccagggtcaagttctcacgatcagcagacgccccggcctaccaacaaggccaaaatcaactgtataatgaactgaaccttgggcggcgagaggagtatgacgttctcgataaacgcagagggcgcgacccagagatgggtgggaaaccgcggcgcaaaaacccccaggaaggactctataatgagctccaaaaggataaaatggctgaagcttacagtgaaatcggaatgaagggtgaacggaggcgagggaagggacacgatggcttgtatcagggactttccacagctacaaaggatacttacgacgccttgcacatgcaggcgcttcctcctcgggggagtggagagggccgcggatcacttctcacatgcggcgacgtagaggaaaatcctggccccatggcagcaccggcgctttcatggcggctgccgctgttgatacttcttttgcctctggcaacatcatgggcttccgccgccgtaaatgggacctcccaatttacttgcttctacaacagtcgcgcgaacatctcctgcgtctggagccaggacggcgctctccaagacacgagctgtcaagtacatgcctggccggatagacgcagatggaatcagacctgcgagttgctccctgtatcccaagcttcatgggcttgcaatctcatcttgggcgccccagatagccaaaaattgactacggtagatattgtcactttgagagtcctctgccgcgaaggcgtcagatggagagtaatggcaattcaagattttaaaccatttgagaatctgcgacttatggcccctatcagtttgcaagtagtacatgtggaaacgcatcgatgcaatatatcatgggagataagccaggcgagtgatttcttcgaaaggcacctcgagtttgaggcccgaactttgagcccaggtcacacttgggaagaagcacccttgctcactcttaaacagaaacaggagtggatatgtttggagacacttactccggacacgcagtatgagttccaagttagagtgaaacccctgcaaggagaattcaccacttggtctccatggagtcaaccattggcttttcgcactaaacccgcggcgcttggaaaggacactataccgtggcttggacatttgctcgtcggtctgagcggagctttcgggtttattatattggtctatttgctcattaattgcaggaatacaggcccgtggctgaagaaggttctcaaatgtaatacacccgacccatctaagtttttctctcaactctcctctgagcacggaggagacgtccaaaagtggctttcaagccccttcccgagtagttcctttagtccgggtggactcgcgccggagattagtcccctggaagtattggaacgagacaaggttactcagctccttctgcagcaagacaaggttcctgaacccgcttccttgtccagtaatcattcactgacatcttgctttaccaaccaaggatatttttttttccatcttcctgacgccctggaaattgaggcttgccaagtgtactttacgtacgacccttattccgaagaagacccagacgagggcgtagcgggtgcaccaactgggtccagccctcaaccgcttcagccgcttagtggggaagacgatgcatattgtactttccccagtagggacgatcttcttctcttttccccaagtctgctcgggggcccgagccctccatcaaccgccccaggcggttcaggcgccggcgaagagagaatgcctccaagccttcaagaaagagttccacgggactgggatcctcagccacttggtccaccgactcccggtgttcctgatctggttgacttccagcctccaccagagcttgtgcttagggaggcaggcgaagaagtgcccgacgctgggccaagggagggagttagtttcccgtggtctagaccgcctggccagggcgagttcagagctttgaacgcgcggctccctttgaataccgacgcttacttgtcattgcaagagctgcaagggcaagacccgacacacttggtgtgataa

SEQ ID NO: 47 - DNA, 8127 bp, synthetic construct
  source 1..8127: mol_type = other DNA
  misc_feature 1..8127: note = Description of sequence:synthetic construct
tttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgaatttaaatcggatccgaccggtggcgcgccctcgagggtaccgctagcacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccgcctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctgggga

SEQ ID NO: 48 - DNA, 3183 bp, synthetic construct
  source 1..3183: mol_type = other DNA
  misc_feature 1..3183: note = Description of sequence:synthetic construct
atggctctgccggttacagccctgctgcttcctctcgccctccttcttcatgcagcgcgaccggatgtcgtgatgacccagagccccctgtctctgcccgtcactccaggtgaaccggcatcaatatcatgtcgaagtagtcaatcactcgttcattcaaacgcaaatacctatctccactggtacctgcagaagcccggacaatcacctcagttgctgatctacaaggtatctaaccgcttctcaggcgttccagatagattctctgggtctggttccgggacagactttacgctcaaaattagccgagttgaggctgaggacgtaggggtgtattattgttcccaaaatactcatgttccgcccaccttcgggcagggcaccaaacttgaaataaagcgcggcagcacttcaggaagcggcaaacctggctctggtgagggaagcacgaagggccaggtacaactcgtacaatcaggagctgaagttaaaaagcccggggcgtcagtcaaagtgtcctgcaaggccagtggctatacgtttactgactatgagatgcattgggtgcgccaggctccgggtcaagggctcgagtggatgggagcacttgatcccaagacgggtgacactgcttactctcagaaatttaaagggcgggttacactcacggcggacgagtccacatcaacggcgtatatggagttgagttcactgcgaagcgaagatacggctgtctactattgcacccgattttattcatacacgtattggggtcaaggtaccctcgtgactgtttcatcaactacaacgcccgctccgcggcccccaactcctgctcccacaatcgccagtcaacctcttagcttgcgccccgaagcatgtagacccgcggccggtggggctgtgcatacacgcggacttgacttcgcctgcgacatttacatttgggctcccttggctggtacttgtggggttctgcttctttctcttgttattactaaacgcggccggaaaaagttgttgtatatctttaagcaacctttcatgaggccggtacagacaacgcaagaggaggatggatgcagttgtagattcccggaagaagaagagggcgggtgtgagcttagggtcaagttctcacgatcagcagacgccccggcctaccaacaaggccaaaatcaactgtataatgaactgaaccttgggcggcgagaggagtatgacgttctcgataaacgcagagggcgcgacccagagatgggtgggaaaccgcggcgcaaaaacccccaggaaggactctataatgagctccaaaaggataaaatggctgaagcttacagtgaaatcggaatgaagggtgaacggaggcgagggaagggacacgatggcttgtatcagggactttccacagctacaaaggatacttacgacgccttgcacatgcaggcgcttcctcctcgggggagtggagagggccgcggatcacttctcacatgcggcgacgtagaggaaaatcctggccccatggcagcaccggcgctttcatggcggctgccgctgttgatacttcttttgcctctggcaacatcatgggcttccgccgccgtaaatgggacctcccaatttacttgcttctacaacagtcgcgcgaacatctcctgcgtctggagccaggacggcgctctccaagacacgagctgtcaagtacatgcctggccggatagacgcagatggaatcagacctgcgagttgctccctgtatcccaagcttcatgggcttgcaatctcatcttgggcgccccagatagccaaaaattgactacggtagatattgtcactttgagagtcctctgccgcgaaggcgtcagatggagagtaatggcaattcaagattttaaaccatttgagaatctgcgacttatggcccctatcagtttgcaagtagtacatgtggaaacgcatcgatgcaatatatcatgggagataagccaggcgagtgatttcttcgaaaggcacctcgagtttgaggcccgaactttgagcccaggtcacacttgggaagaagcacccttgctcactcttaaacagaaacaggagtggatatgtttggagacacttactccggacacgcagtatgagttccaagttagagtgaaacccctgcaaggagaattcaccacttggtctccatggagtcaaccattggcttttcgcactaaacccgcggcgcttggaaaggacactataccgtggcttggacatttgctcgtcggtctgagcggagctttcgggtttattatattggtctatttgctcattaattgcaggaatacaggcccgtggctgaagaaggttctcaaatgtaatacacccgacccatctaagtttttctctcaactctcctctgagcacggaggagacgtccaaaagtggctttcaagccccttcccgagtagttcctttagtccgggtggactcgcgccggagattagtcccctggaagtattggaacgagacaaggttactcagctccttctgcagcaagacaaggttcctgaacccgcttccttgtccagtaatcattcactgacatcttgctttaccaaccaaggatatttttttttccatcttcctgacgccctggaaattgaggcttgccaagtgtactttacgtacgacccttattccgaagaagacccagacgagggcgtagcgggtgcaccaactgggtccagccctcaaccgcttcagccgcttagtggggaagacgatgcatattgtactttccccagtagggacgatcttcttctcttttccccaagtctgctcgggggcccgagccctccatcaaccgccccaggcggttcaggcgccggcgaagagagaatgcctccaagccttcaagaaagagttccacgggactgggatcctcagccacttggtccaccgactcccggtgttcctgatctggttgacttccagcctccaccagagcttgtgcttagggaggcaggcgaagaagtgcccgacgctgggccaagggagggagttagtttcccgtggtctagaccgcctggccagggcgagttcagagctttgaacgcgcggctccctttgaataccgacgcttacttgtcattgcaagagctgcaagggcaagacccgacacacttggtgtgataa

SEQ ID NO: 49 - protein, 15 aa, Homo sapiens
  REGION 1..15: note = Description of sequence: G4S x 3 scFv Linker
GGGGSGGGGSGGGGS

SEQ ID NO: 50 - protein, 11 aa, Homo sapiens
  REGION 1..11: note = Description of sequence: albumin-binding peptide
DICLPRWGCLW